(19) United States
(12) Patent Application Publication
(10) Pub. No.: US 2003/0069404 A1
(43) Pub. Date: Apr. 10, 2003

(54) Helicobacter antigens and corresponding DNA fragments

(76) Inventors: Rainer Haas, Tuebingen (DE); Harold Kleanthous, Newtonville, MA (US); Thomas F. Meyer, Tuebingen (DE); Stefan Odenbreit, Ammerbuch (DE); Amal A. Al-Garawi, Boston, MA (US); Charles A. Miller, Medford, MA (US)

Correspondence Address: CLARK & ELBING LLP, 101 FEDERAL STREET, BOSTON, MA, 02110, US

(21) Appl. No.: 10/013,315

(22) Filed: Nov. 5, 2001

Related U.S. Application Data
(63) Continuation of application No. 08/749,051, filed on Nov. 14, 1996, now abandoned.

(51) Int. Cl. C07H 21/02; C07H 21/04

(52) U.S. Cl. 536/23.1

(57) ABSTRACT

The invention provides Helicobacter polypeptides that can be used in vaccination methods for preventing or treating Helicobacter infection, and polynucleotides that encode these polypeptides.

### PRIORITY INFORMATION

This application is a continuation of, and claims priority from, U.S. Ser. No. 08/749,051, filed Nov. 14, 1996, which is incorporated by reference herein.

### FIELD OF THE INVENTION

The invention relates to Helicobacter antigens and corresponding DNA molecules, which can be used in methods to prevent and treat Helicobacter infection in mammals, such as humans.

### BACKGROUND OF THE INVENTION

Helicobacter is a genus of spiral, gram-negative bacteria that colonize the gastrointestinal tracts of mammals. Several species colonize the stomach, most notably H. pylori, H. heilmanii, H. felis, and H. mustelae. Although H. pylori is the species most commonly associated with human infection, H. heilmanii and H. felis have also been isolated from humans, but at lower frequencies than H. pylori. Helicobacter infects over 50% of adult populations in developed countries and nearly 100% in developing countries and some Pacific rim countries, making it one of the most prevalent infections worldwide.

Helicobacter is routinely recovered from gastric biopsies of humans with histological evidence of gastritis and peptic ulceration. Indeed, H. pylori is now recognized as an important pathogen of humans, in that the chronic gastritis it causes is a risk factor for the development of peptic ulcer diseases and gastric carcinoma. It is thus highly desirable to develop safe and effective vaccines for preventing and treating Helicobacter infection.

A number of Helicobacter antigens have been characterized or isolated. These include urease, which is composed of two structural subunits of approximately 30 and 67 kDa (Hu et al., Infect. Immun. 58:992, 1990; Dunn et al., J. Biol. Chem. 265:9464, 1990; Evans et al., Microbial Pathogenesis 10:15, 1991; Labigne et al., J. Bact., 173:1920, 1991); the 87 kDa vacuolar cytotoxin (VacA) (Cover et al., J. Biol. Chem. 267:10570, 1992; Phadnis et al., Infect. Immun. 62:1557, 1994; WO 93/18150); a 128 kDa immunodominant antigen associated with the cytotoxin (CagA, also called TagA) (WO 93/18150; U.S. Pat. No. 5,403,924); 13 and 58 kDa heat shock proteins HspA and HspB (Suerbaum et al., Mol. Microbiol. 14:959, 1994; WO 93/18150); a 54 kDa catalase (Hazell et al., J. Gen. Microbiol. 137:57, 1991); a 15 kDa histidine-rich protein (Hpn) (Gilbert et al., Infect. Immun. 63:2682, 1995); a 20 kDa membrane-associated lipoprotein (Kostrcynska et al., J. Bact. 176:5938, 1994), an 30 kDa outer membrane protein (Bölin et al., J. Clin. Microbiol. 33:381, 1995); a lactoferrin receptor (FR 2,724,936), and several porins, referred to as HopA, HopB, HopC, HopD, and HopE, which have molecular weights of 48-67 kDa (Exner et al., Infect. Immun. 63:1567, 1995; Doig et al., J. Bact. 177:5447, 1995).

Some of these proteins have been proposed as potential vaccine antigens. In particular, urease is believed to be a vaccine candidate (WO 94/9823; WO 95/22987; WO 95/3824; Michetti et al., Gastroenterology 107:1002, 1994). Nevertheless, it is contemplated that several antigens may ultimately be necessary in a vaccine.

### SUMMARY OF THE INVENTION

The present invention provides DNA molecules that encode Helicobacter polypeptides designated HPO101, HPO104, HPO116, HPO121, HPO132, HPO15, HPO18, HPO38, HPO42, HPO45, HPO50, HPO54, HPO57, HPO58, HPO64, HPO70, HPO71, HPO76, HPO7, HPO80, HPO87, HPO95, HPO98, and HPO9, which can be used in methods to prevent, treat, and diagnose Helicobacter infection. The encoded polypeptides include polypeptides having the amino acid sequences shown in SEQ ID NOs:2 to 48 (even numbers) and polypeptides encoded by DNA inserts found in deposited plasmids (see below, e.g., Example 2). Those skilled in the art will appreciate that the invention also includes DNA molecules that encode mutants and derivatives of such polypeptides, which result from the addition, deletion, or substitution of non-essential amino acids as described herein. The invention also includes RNA molecules corresponding to the DNA molecules of the invention.

In addition to the DNA and RNA molecules, the invention includes the corresponding polypeptides and monospecific antibodies that specifically bind to such polypeptides.

The present invention has wide application and includes expression cassettes, vectors, and cells transformed or transfected with the polynucleotides of the invention. Accordingly, the present invention provides (i) a method for producing a polypeptide of the invention in a recombinant host system and related expression cassettes, vectors, and transformed or transfected cells; (ii) a live vaccine vector, such as a pox virus, Salmonella typhimurium, or Vibrio cholerae vector, containing a polynucleotide of the invention, such vaccine vectors being useful for, e.g., preventing and treating Helicobacter infection, in combination with a diluent or carrier, and related pharmaceutical compositions and associated therapeutic and/or prophylactic methods; (iii) a therapeutic and/or prophylactic method involving administration of an RNA or DNA molecule of the invention, either in a naked form or formulated with a delivery vehicle, a polypeptide or combination of polypeptides, or a monospecific antibody of the invention, and related pharmaceutical compositions; (iv) a method for diagnosing the presence of Helicobacter in a biological sample, which can involve the use of a DNA or RNA molecule, a monospecific antibody, or a polypeptide of the invention; and (v) a method for purifying a polypeptide of the invention by antibody-based affinity chromatography.

### BRIEF DESCRIPTION OF THE DRAWINGS

FIG. 1A is a diagrammatic representation of transposon TnMax9, which is a derivative of the TnMax transposon system (Haas et al., Gene 130:23-21, 1993). The mini-transposon carries the blaM gene, which is the β-lactamase gene lacking a promoter and a signal sequence, next to the inverted repeats (IR) and the M13 forward (M13-FP) and reverse (M13-RP1) primer binding sites. The resolution site (res) and an origin of replication (ori_{fd}) are located between the blaM gene and the constitutive cat_{GC}-resistance gene. The transposase tnpA and resolvase tnpR genes are located outside of the mini-transposon and are under the control of the inducible P_{trc} promoter. The lacIq gene encodes the Lac repressor.

FIG. 1B is a diagrammatic representation of plasmid pMin2. pMin2 contains a multiple cloning site, the tetracycline resistance gene (tet), an origin of transfer (oriT), an origin of replication (ori_{ColE1}), a transcriptional terminator (t_{fd}), and a weak, constitutive promoter (P_{iga}). H. pylori chromosome fragments were introduced into the BglII and ClaI sites of pMin2.

FIGS. 2A-2E are a series of graphs showing an analysis of some of the physical properties of polypeptide HPO76 (SEQ ID NO:36).

FIGS. 3A-3D are a series of graphs showing an analysis of some of the physical properties of polypeptide HPO15 (SEQ ID NO:12).

FIGS. 4A-4F are a series of graphs showing an analysis of some of the physical properties of polypeptide HPO42 (SEQ ID NO:18).

FIGS. 5A-5D are a series of graphs showing an analysis of some of the physical properties of polypeptide HPO50 (SEQ ID NO:22).

FIGS. 6A-6H are a series of graphs showing an analysis of some of the physical properties of polypeptide HPO54 (SEQ ID NO:24).

FIGS. 7A-7G are a series of graphs showing an analysis of some of the physical properties of polypeptide HPO57 (SEQ ID NO:26).

FIGS. 8A-8G are a series of graphs showing an analysis of some of the physical properties of polypeptide HPO64 (SEQ ID NO:30).

### DETAILED DESCRIPTION

In the H. pylori genome, open reading frames (ORFs) encoding full length, membrane-associated secreted/excreted polypeptides have been newly identified. These polypeptides include membrane polypeptides permanently found in the membrane structure and polypeptides that are present in the external vicinity of the membrane. These polypeptides can be used in vaccination methods for preventing and treating Helicobacter infection. The ORFs encode secreted polypeptides that can be readily produced in their mature form (polypeptides exported through class II or III secretion pathway) or are initially produced as precursors including a signal peptide that can be removed in the course of excretion/secretion by cleavage at the N-terminal end of the mature form. (The cleavage site is located at the C-terminal end of the signal peptide, adjacent to the mature form.) In the sequences disclosed in the present application, these cleavage sites and accordingly the first amino acid of the mature polypeptides, were putatively determined.

According to a first aspect of the invention, there are provided isolated polynucleotides encoding the precursor and mature forms of Helicobacter HPO101, HPO104, HPO116, HPO121, HPO132, HPO15, HPO18, HPO38, HPO42, HPO45, HPO50, HPO54, HPO57, HPO58, HPO64, HPO70, HPO71, HPO76, HPO7, HPO80, HPO87, HPO95, HPO98, and HPO9.

An isolated polynucleotide of the invention encodes (i) a polypeptide having an amino acid sequence that is homologous to a Helicobacter amino acid sequence of a polypeptide associated with the Helicobacter membrane, the Helicobacter amino acid sequence being selected from the group consisting of:

(a) the amino acid sequences as shown:

in SEQ ID NO:2, beginning with an amino acid in any one of the positions from −27 to 5, preferably in position −27 or position 1, and ending with an amino acid in position 160 (HPO101);

in SEQ ID NO:4, beginning with an amino acid in position 1 and ending with an amino acid in position 172 (HPO104);

in SEQ ID NO:6, beginning with an amino acid in any one of the positions from −17 to 5, preferably in position −17 or position 1, and ending with an amino acid in position 169 (HPO116);

in SEQ ID NO:8, beginning with an amino acid in any one of the positions from −21 to 5, preferably in position −20 or position 1, and ending with an amino acid in position 198 (HPO121);

in SEQ ID NO:10, beginning with an amino acid in any one of the positions from −20 to 5, preferably in position −20 or position 1, and ending with an amino acid in position 132 (HPO132);

in SEQ ID NO:12, beginning with an amino acid in 1 to 5, preferably in position 1, and ending with an amino acid in position 114 (HPO15);

in SEQ ID NO:14, beginning with an amino acid in any one of the positions from −17 to 5, preferably in position −17 or position 1, and ending with an amino acid in position 248 (HPO18);

in SEQ ID NO:16, beginning with an amino acid in any one of the positions from −40 to 5, preferably in position −40 or position 1, and ending with an amino acid in position 74 (HPO38);

in SEQ ID NO:18, beginning with an amino acid in any one of the positions from −34 to 5, preferably in position −34 or position 1, and ending with an amino acid in position 226 (HPO42);

in SEQ ID NO:20, beginning with an amino acid in any one of the positions from −21 to 5, preferably in position −21 or position 1, and ending with an amino acid in position 179 (HPO45);

in SEQ ID NO:22, beginning with an amino acid in any one of the positions from −33 to 5, preferably in position −33 or position 1, and ending with an amino acid in position 114 (HPO50);

in SEQ ID NO:24, beginning with an amino acid in any one of the positions from −60 to 5, preferably in position −60 or position 1, and ending with an amino acid in position 349 (HPO54);

in SEQ ID NO:26, beginning with an amino acid in any one of the positions from −18 to 5, preferably in position −18 or position 1, and ending with an amino acid in position 288 (HPO57);

in SEQ ID NO:28, beginning with an amino acid in any one of the positions from −21 to 5, preferably in position −21 or position 1, and ending with an amino acid in position 150 (HPO58);

in SEQ ID NO:30, beginning with an amino acid in any one of the positions from −20 to 5, preferably in position −20 or position 1, and ending with an amino acid in position 309 (HPO64);

in SEQ ID NO:32, beginning with an amino acid in any one of the positions from −35 to 5, preferably in position −35 or position 1, and ending with an amino acid in position 129 (HPO70);

in SEQ ID NO:34, beginning with an amino acid in any one of the positions from −19 to 5, preferably in position −19 or position 1, and ending with an amino acid in position 153 (HPO71);

in SEQ ID NO:36, beginning with an amino acid in any one of the positions from −25 to 5, preferably in position −25 or position 1, and ending with an amino acid in position 176 (HPO76);

in SEQ ID NO:38, beginning with an amino acid in any one of the positions from −21 to 5, preferably in position −21 or position 1, and ending with an amino acid in position 156 (HPO7);

in SEQ ID NO:40, beginning with an amino acid in position 1 and ending with an amino acid in position 144 (HPO80);

in SEQ ID NO:42, beginning with an amino acid in any one of the positions from −20 to 5, preferably in position −20 or position 1, and ending with an amino acid in position 152 (HPO87);

in SEQ ID NO:44, beginning with an amino acid in any one of the positions from −31 to 5, preferably in position −31 or position 1, and ending with an amino acid in position 112 (HPO95);

in SEQ ID NO:46, beginning with an amino acid in any one of the positions from −20 to 5, preferably in position −20 or position 1, and ending with an amino acid in position 91 (HPO98);

in SEQ ID NO:48, beginning with an amino acid in any one of the positions from −21 to 5, preferably in position −21 or position 1, and ending with an amino acid in position 129 (HPO9); and

(b) the precursor or mature amino acid sequences encoded by the H. pylori DNA inserts found in American Type Culture Collection deposit numbers HPO76 (98197), HPO18 (98210), HPO121 (98201), HPO45 (98208), HPO101 (98198), HPO116 (98200), HPO7 (98211), HPO104 (98199), HPO15 (98214), HPO58 (98206), HPO132 (98202), HPO9 (98203), HPO38 (98204), HPO87 (98205), HPO71 (98217), HPO70 (98219), HPO80 (98215), HPO95 (98216), HPO98 (98218), HPO57 (98220), HPO50 (98207), HPO64 (98213), HPO54 (98212), and HPO42 (98209); or (ii) a derivative of the polypeptide.

The term “isolated polynucleotide” is defined as a polynucleotide removed from the environment in which it naturally occurs. For example, a naturally-occurring DNA molecule present in the genome of a living bacteria or as part of a gene bank is not isolated, but the same molecule separated from the remaining part of the bacterial genome, as a result of, e.g., a cloning event (amplification), is isolated. Typically, an isolated DNA molecule is free from DNA regions (e.g., coding regions) with which it is immediately contiguous at the 5′ or 3′ end, in the naturally occurring genome. Such isolated polynucleotides could be part of a vector or a composition and still be isolated in that such a vector or composition is not part of its natural environment.

A polynucleotide of the invention can be in the form of RNA or DNA (e.g., cDNA, genomic DNA, or synthetic DNA), or modifications or combinations thereof. The DNA can be double-stranded or single-stranded, and, if single-stranded, can be the coding strand or the non-coding (anti-sense) strand. The sequence that encodes a polypeptide of the invention as shown in SEQ ID NOs:2 to 48 (even numbers), or encoded by a deposited DNA molecule, can be (a) the coding sequence as shown in SEQ ID NOs:1 to 47 (odd numbers), (b) the coding sequence of a deposited DNA molecule of the invention (see below); (c) a ribonucleotide sequence derived by transcription of (a) or (b); or (d) a different coding sequence; this latter, as a result of the redundancy or degeneracy of the genetic code, encodes the same polypeptides as the DNA molecules of which the nucleotide sequences are illustrated in SEQ ID NOs:1 to 47 (odd numbers) or the deposited DNA molecules of the invention.

Advantageously, the polypeptide is naturally secreted or excreted by Helicobacter felis, H. mustelae, H. heilmanii, or H. pylori; the latter being preferred.

By “polypeptide” or “protein” is meant any chain of amino acids, regardless of length or post-translational modification (e.g., glycosylation or phosphorylation). Both terms are used interchangeably in the present application.

By “homologous amino acid sequence” is meant an amino acid sequence that differs from an amino acid sequence shown in SEQ ID NOs:2-48 (even numbers) or encoded by a deposited DNA molecule of the invention, only by one or more conservative amino acid substitutions, or by one or more non-conservative amino acid substitutions, deletions, or additions located at positions at which they do not destroy the specific antigenicity of the polypeptide.

Preferably, such a sequence is at least 75%, more preferably 80%, and most preferably 90% identical to an amino acid sequence shown in SEQ ID NOs:2 to 48 (even numbers) or encoded by a deposited DNA molecule of the invention.

Homologous amino acid sequences include sequences that are identical or substantially identical to an amino acid sequence as shown in SEQ ID NOs:2 to 48 (even numbers) or encoded by a deposited DNA molecule of the invention. By “amino acid sequence substantially identical” is meant a sequence that is at least 90%, preferably 95%, more preferably 97%, and most preferably 99% identical to an amino acid sequence of reference and that preferably differs from the sequence of reference, if at all, by a majority of conservative amino acid substitutions.

Conservative amino acid substitutions typically include substitutions among amino acids of the same class. These classes include, for example, amino acids having uncharged polar side chains, such as asparagine, glutamine, serine, threonine, and tyrosine; amino acids having basic side chains, such as lysine, arginine, and histidine; amino acids having acidic side chains, such as aspartic acid and glutamic acid; and amino acids having nonpolar side chains, such as glycine, alanine, valine, leucine, isoleucine, proline, phenylalanine, methionine, tryptophan, and cysteine.

As an illustration of substitutive variations, particular examples are provided as follows. In the sequence shown in SEQ ID NO:4, the lysine in position 96 can be substituted with asparagine, glutamine, isoleucine, threonine, glutamic acid, or arginine; the asparagines in positions 120 and 123 can be substituted with isoleucine, threonine, lysine, serine, tyrosine, or asparagine; the lysines in positions 125, 128, and 144 can be substituted with asparagine, glutamine, isoleucine, threonine, glutamic acid, or arginine; or the proline in position 150 can be substituted with serine, threonine, alanine, leucine, arginine, or histidine. In the sequence shown in SEQ ID NO:8, the leucine in position 115 can be substituted with phenylalanine, isoleucine, valine, proline, histidine, or arginine. In the sequence shown in SEQ ID NO:10, the arginine in position 107 can be substituted with glycine, the asparagine in position 118 can be substituted with isoleucine, threonine, or serine; or the proline in position 130 can be substituted with serine, threonine, alanine, leucine, arginine, or histidine. In the sequence shown in SEQ ID NO:12, the asparagine in position 17 can be substituted with isoleucine, threonine, or serine. In the sequence shown in SEQ ID NO:12, the asparagine in position 17 can be , substituted with isoleucine, threonine, or serine. In the sequence shown in SEQ ID NO:40, the asparagine in position 33 can be substituted with isoleucine, threonine, or serine, and the phenylalanine in position 128 can be substituted with serine, tyrosine, or cysteine. In the sequence shown in SEQ ID NO:50, the glutamine in position 10 can be substituted with leucine, proline, or arginine; the leucine in position 26 can be substituted with phenylalanine, and the arginine in position 127 can be substituted with glycine.

Homology is typically measured using sequence analysis software (e.g., Sequence Analysis Software Package of the Genetics Computer Group, University of Wisconsin Biotechnology Center, 1710 University Avenue, Madison, Wis. 53705). Similar amino acid sequences are aligned to obtain the maximum degree of homology (i.e., identity). To this end, it may be necessary to artificially introduce gaps into the sequence. Once the optimal alignment has been set up, the degree of homology (i.e., identity) is established by recording all of the positions in which the amino acids of both sequences are identical, relative to the total number of positions.

Homologous polynucleotide sequences are defined in a similar way. Preferably, a homologous sequence is one that is at least 45%, more preferably 60%, and most preferably 85% identical to (i) a coding sequence of SEQ ID NOs:1 to 47 (odd numbers), or (ii) a coding sequence of a deposited DNA molecule of the invention.

Polypeptides having a sequence homologous to one of the sequences shown in SEQ ID NOs:2 to 48 (even numbers), include naturally-occurring allelic variants, as well as mutants or any other non-naturally occurring variants that are analogous in terms of antigenicity, to a polypeptide having a sequence as shown in SEQ ID NOs:2 to 48 (even numbers) or encoded by a deposited DNA molecule of the invention.

As is known in the art, an allelic variant is an alternate form of a polypeptide that is characterized as having a substitution, deletion, or addition of one or more amino acids that does not alter the biological function of the polypeptide. By “biologic function” is meant the function of the polypeptide in the cells in which it naturally occurs, even if the function is not necessary for the growth or survival of the cells. For example, the biological function of a porin is to allow the entry into cells of compounds present in the extracellular medium. The biological function is distinct from the antigenic function. A polypeptide can have more than one biological function.

Allelic variants are very common in nature. For example, a bacterial species, e.g., H. pylori, is usually represented by a variety of strains that differ from each other by minor allelic variations. Indeed, a polypeptide that fulfills the same biological function in different strains can have an amino acid sequence that is not identical in each of the strains. Such an allelic variation may be equally reflected at the polynucleotide level.

Support for the use of allelic variants of polypeptide antigens comes from, e.g., studies of the Helicobacter urease antigen. The amino acid sequence of Helicobacter urease varies widely from species to species, yet cross-species protection occurs, indicating that the urease molecule, when used as an immunogen, is highly tolerant of amino acid variations. Even among different strains of the single species H. pylori, there are amino acid sequence variations.

For example, although the amino acid sequences of the UreA and UreB subunits of H. pylori and H. felis ureases differ from one another by 26.5% and 11.8%, respectively (Ferrero et al., Molecular Microbiology 9(2):323-333, 1993), it has been shown that H. pylori urease protects mice from H. felis infection (Michetti et al., Gastroenterology 107:1002-1011, 1994). In addition, it has been shown that the individual structural subunits of urease, UreA and UreB, which contain distinct amino acid sequences, are both protective antigens against Helicobacter infection (Michetti et al., supra). Similarly, Cuenca et al. (Gastroenterology 110: 1770-1775, 1996) showed that therapeutic immunization of H. mustelae-infected ferrets with H. pylori urease was effective at eradicating H. mustelae infection. Further, several urease variants have been reported to be effective vaccine antigens, including, e.g., recombinant UreA+UreB apoenzyme expressed from pORV142 (UreA and UreB sequences derived from H. pylori strain CPM630; Lee et al., J. Infect. Dis. 172:161-172, 1995); recombinant UreA+UreB apoenzyme expressed from pORV214 (UreA and UreB sequences differ from H. pylori strain CPM630 by one and two amino acid changes, respectively; Lee et al., supra, 1995); a UreA-glutathione-S-transferase fusion protein (UreA sequence from H. pylori strain ATCC 43504; Thomas et al., Acta Gastro-Enterologica Belgica, 56:54, September 1993); UreA+UreB holoenzyme purified from H. pylori strain NCTC11637 (Marchetti et al., Science 267:1655-1658, 1995); a UreA-MBP fusion protein (UreA from H. pylori strain 85P; Ferrero et al., Infection and Immunity 62:4981-4989, 1994); a UreB-MBP fusion protein (UreB from H. pylori strain 85P; Ferrero et al., supra); a UreA-MBP fusion protein (UreA from H. felis strain ATCC 49179; Ferrero et al., supra); a UreB-MBP fusion protein (UreB from H. felis strain ATCC 49179; Ferrero et al., supra); and a 37 kD fragment of UreB containing amino acids 220-569 (Dore-Davin et al., “A 37 kD fragment of UreB is sufficient to confer protection against Helicobacter felis infection in mice”). Finally, Thomas et al. (supra) showed that oral immunization of mice with crude sonicates of H. pylori protected mice from subsequent challenge with H. felis.

Polynucleotides, e.g., DNA molecules, encoding allelic variants can easily be retrieved by polymerase chain reaction (PCR) amplification of genomic bacterial DNA extracted by conventional methods. This involves the use of synthetic oligonucleotide primers matching upstream and downstream of the 5′ and 3′ ends of the encoding domain. Suitable primers can be designed according to the nucleotide sequence information provided in SEQ ID NOs:1 to 47 (odd numbers). Typically, a primer can consist of 10 to 40, preferably 15 to 25 nucleotides. It may be also advantageous to select primers containing C and G nucleotides in a proportion sufficient to ensure efficient hybridization; e.g., an amount of C and G nucleotides of at least 40%, preferably 50% of the total nucleotide amount.

As an example, primers useful for cloning by PCR a DNA molecule encoding a polypeptide having the amino acid sequence of HPO76 (SEQ ID NO:36), or encoded by the corresponding deposited DNA molecule (pMin2/76; HPO76, ATCC Deposit Number 98197), are shown in SEQ ID NO:83 (matching at the 5′ end) and in SEQ ID NO:84 (matching at the 3′ end). Experimental conditions for carrying out PCR can readily be determined by one skilled in the art and an illustration of carrying out PCR is provided in Example 1.

Thus, the first aspect of the invention includes (i) isolated DNA molecules that can be amplified and/or cloned by polymerase chain reaction from a Helicobacter, e.g., H. pylori, genome, using either:

A 5′ oligonucleotide primer having a sequence as shown in SEQ ID NO:49, and a 3′ oligonucleotide primer having a sequence in SEQ ID NO:50;

A 5′ oligonucleotide primer having a sequence as shown in SEQ ID NO:51, and a 3′ oligonucleotide primer having a sequence in SEQ ID NO:52;

A 5′ oligonucleotide primer having a sequence as shown in SEQ ID NO:53, and a 3′ oligonucleotide primer having a sequence in SEQ ID NO:54;

A 5′ oligonucleotide primer having a sequence as shown in SEQ ID NO:55, and a 3′ oligonucleotide primer having a sequence in SEQ ID NO:56;

A 5′ oligonucleotide primer having a sequence as shown in SEQ ID NO:57, and a 3′ oligonucleotide primer having a sequence in SEQ ID NO:58;

A 5′ oligonucleotide primer having a sequence as shown in SEQ ID NO:59, and a 3′ oligonucleotide primer having a sequence in SEQ ID NO:60;

A 5′ oligonucleotide primer having a sequence as shown in SEQ ID NO:61, and a 3′ oligonucleotide primer having a sequence in SEQ ID NO:62;

A 5′ oligonucleotide primer having a sequence as shown in SEQ ID NO:63, and a 3′ oligonucleotide primer having a sequence in SEQ ID NO:64;

A 5′ oligonucleotide primer having a sequence as shown in SEQ ID NO:65, and a 3′ oligonucleotide primer having a sequence in SEQ ID NO:66;

A 5′ oligonucleotide primer having a sequence as shown in SEQ ID NO:67, and a 3′ oligonucleotide primer having a sequence in SEQ ID NO:68;

A 5′ oligonucleotide primer having a sequence as shown in SEQ ID NO:69, and a 3′ oligonucleotide primer having a sequence in SEQ ID NO:70;

A 5′ oligonucleotide primer having a sequence as shown in SEQ ID NO:71, and a 3′ oligonucleotide primer having a sequence in SEQ ID NO:72;

A 5′ oligonucleotide primer having a sequence as shown in SEQ ID NO:73, and a 3′ oligonucleotide primer having a sequence in SEQ ID NO:74;

A 5′ oligonucleotide primer having a sequence as shown in SEQ ID NO:75, and a 3′ oligonucleotide primer having a sequence in SEQ ID NO:76;

A 5′ oligonucleotide primer having a sequence as shown in SEQ ID NO:77, and a 3′ oligonucleotide primer having a sequence in SEQ ID NO:78;

A 5′ oligonucleotide primer having a sequence as shown in SEQ ID NO:79, and a 3′ oligonucleotide primer having a sequence in SEQ ID NO:80;

A 5′ oligonucleotide primer having a sequence as shown in SEQ ID NO:81, and a 3′ oligonucleotide primer having a sequence in SEQ ID NO:82;

A 5′ oligonucleotide primer having a sequence as shown in SEQ ID NO:83, and a 3′ oligonucleotide primer having a sequence in SEQ ID NO:84;

A 5′ oligonucleotide primer having a sequence as shown in SEQ ID NO:85, and a 3′ oligonucleotide primer having a sequence in SEQ ID NO:86;

A 5′ oligonucleotide primer having a sequence as shown in SEQ ID NO:87, and a 3′ oligonucleotide primer having a sequence in SEQ ID NO:88;

A 5′ oligonucleotide primer having a sequence as shown in SEQ ID NO:89, and a 3′ oligonucleotide primer having a sequence in SEQ ID NO:90;

A 5′ oligonucleotide primer having a sequence as shown in SEQ ID NO:91, and a 3′ oligonucleotide primer having a sequence in SEQ ID NO:93;

A 5′ oligonucleotide primer having a sequence as shown in SEQ ID NO:95, and a 3′ oligonucleotide primer having a sequence in SEQ ID NO:94;

A 5′ oligonucleotide primer having a sequence as shown in SEQ ID NO:97, and a 3′ oligonucleotide primer having a sequence in SEQ ID NO:96; or

A 5′ oligonucleotide primer having a sequence as shown in SEQ ID NO:99, and a 3′ oligonucleotide primer having a sequence in SEQ ID NO:98; and

(ii) isolated DNA molecules encoding the mature forms of the polypeptides encoded by the DNA molecules amplified as above.

In the sequences provided in SEQ ID NOs:49 to 96, the letter “N” denotes a restriction site that contains, typically, 4 to 6 nucleotides. Restriction sites can be selected by those skilled in the art so that the amplified DNA can be conveniently cloned into an appropriately digested plasmid.

Useful homologs that do not naturally occur can be designed using known methods for identifying regions of an antigen that are likely to be tolerant of amino acid sequence changes and/or deletions. For example, sequences of the antigen from different species can be compared to identify conserved sequences.

Polypeptide derivatives that are encoded by polynucleotides of the invention include, e.g., fragments, polypeptides having large internal deletions derived from full-length polypeptides, and fusion proteins.

Polypeptide fragments of the invention can be derived from a polypeptide having a sequence homologous to any of the sequences shown in SEQ ID NOs:2 to 48 (even numbers) or encoded by a deposited DNA molecule of the invention (see below, e.g., Example 2), to the extent that the fragments retain the substantial antigenicity of the parent polypeptide (specific antigenicity). Polypeptide derivatives can also be constructed by large internal deletions that remove a substantial part of the parent polypeptide, while retaining specific antigenicity. Generally, polypeptide derivatives should be about at least 12 amino acids in length to maintain antigenicity. Advantageously, they can be at least 20 amino acids, preferably at least 50 amino acids, more preferably at least 75 amino acids, and most preferably at least 100 amino acids in length.

Useful polypeptide derivatives, e.g., polypeptide fragments, can be designed using computer-assisted analysis of amino acid sequences in order to identify sites in protein antigens having potential as surface-exposed, antigenic regions (Hughes et al., Infect. Immun. 60(9):3497, 1992).

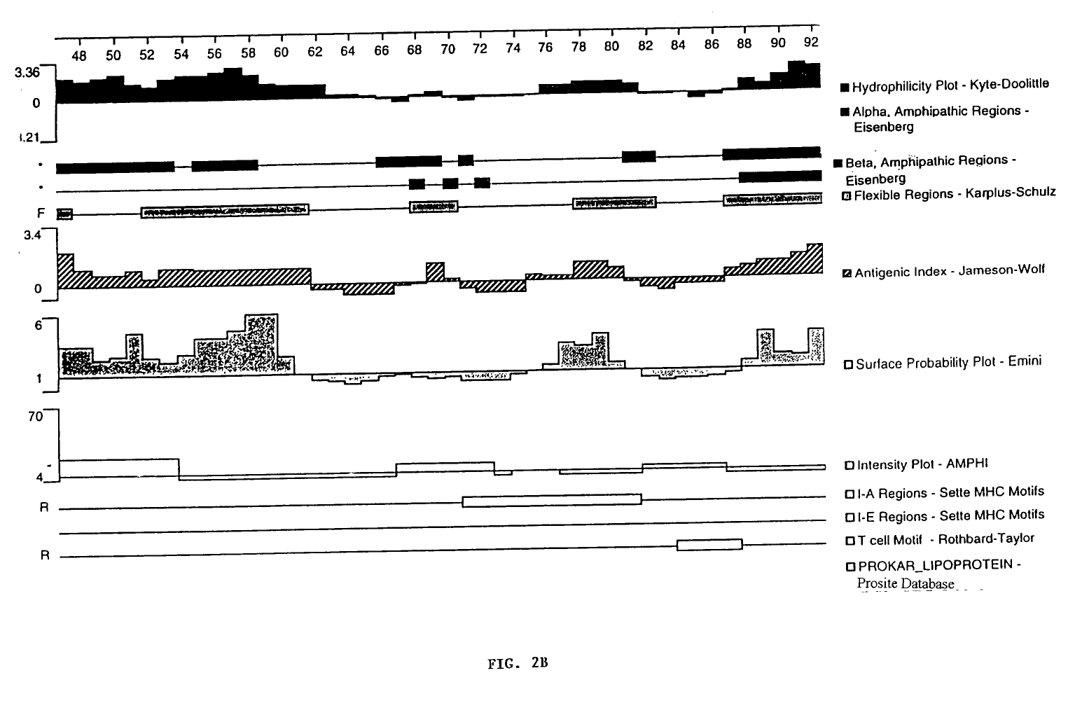

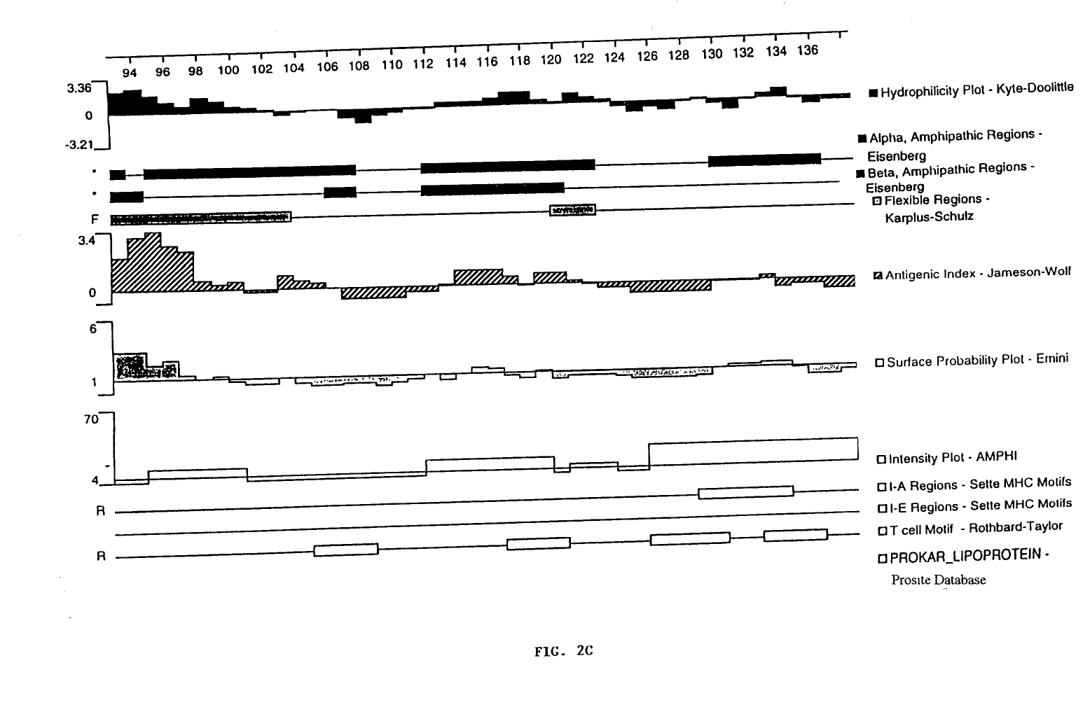

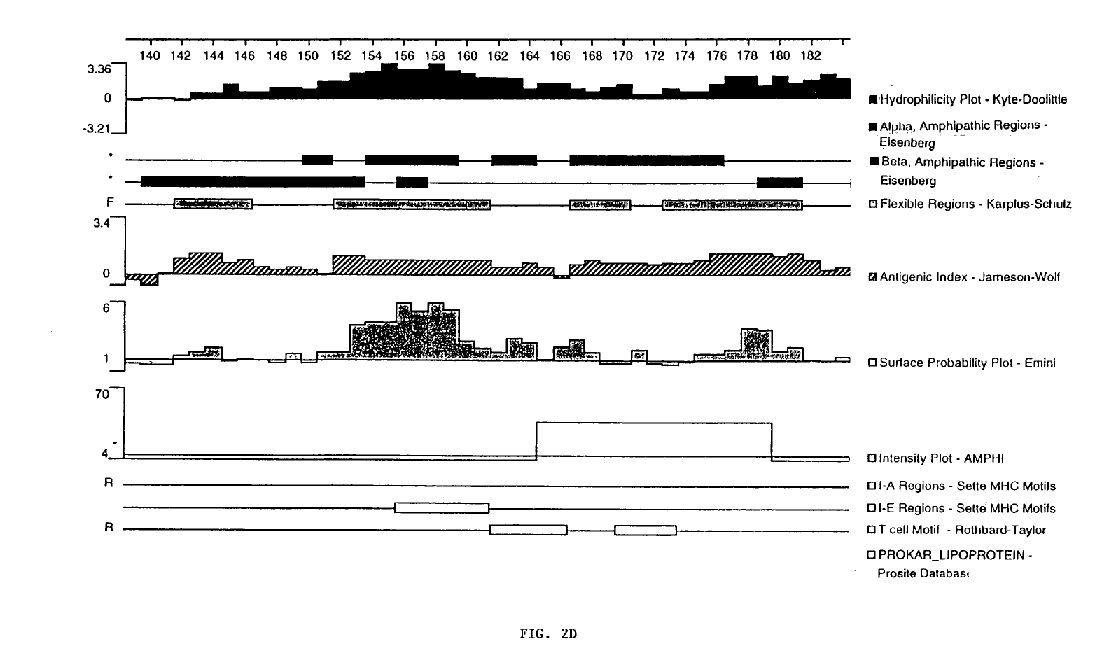

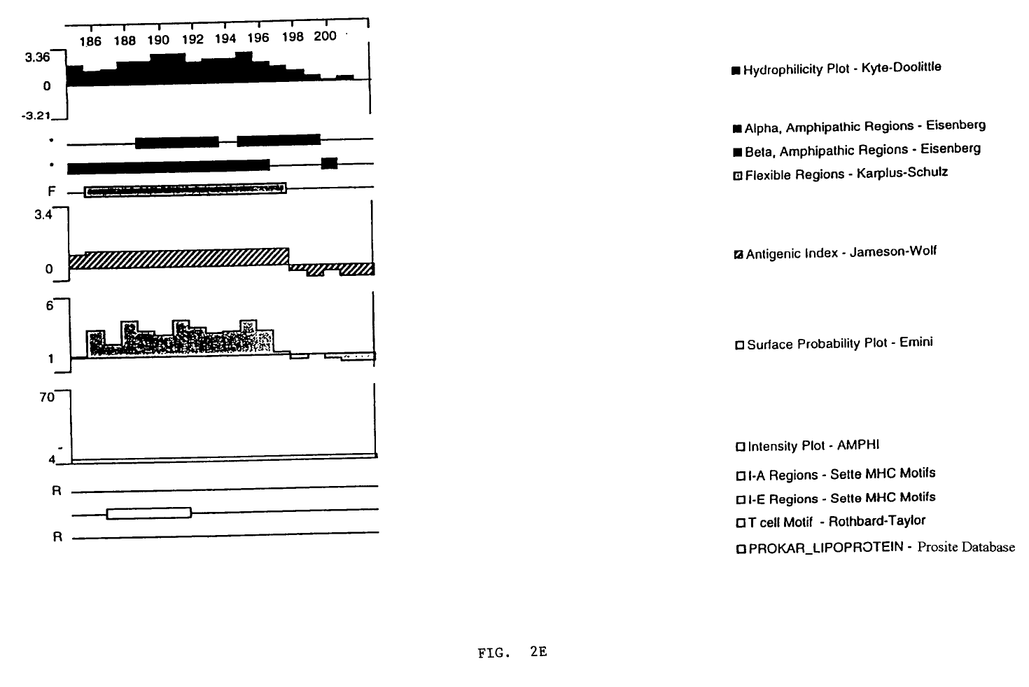

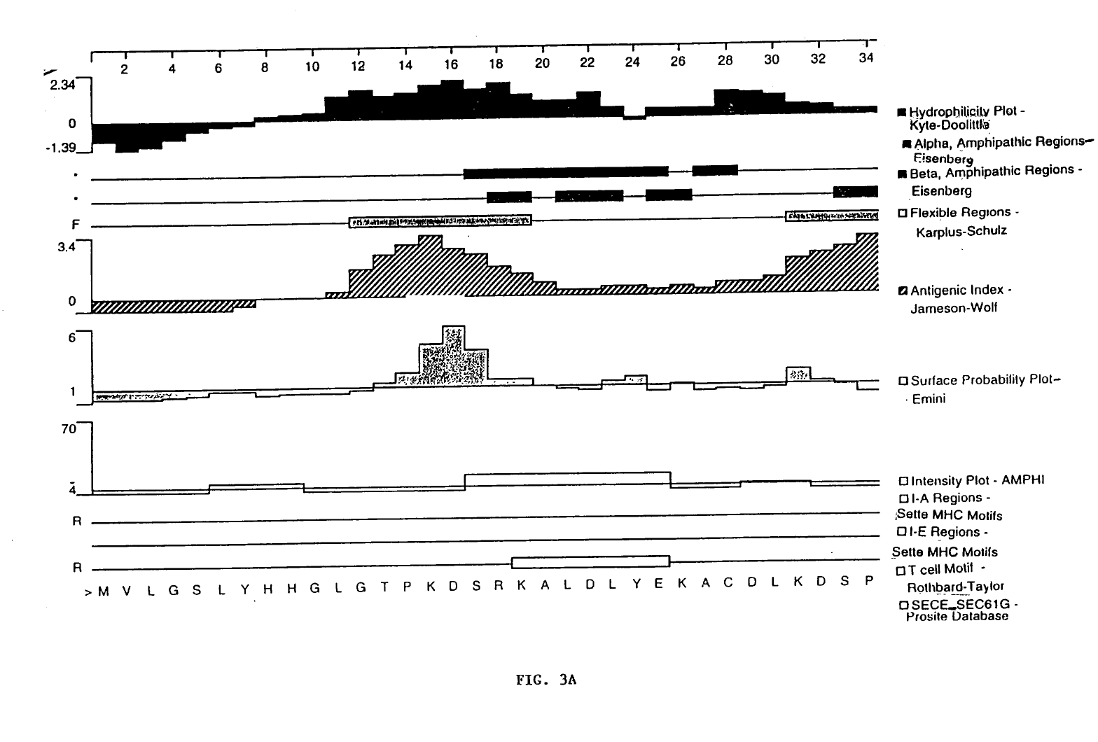

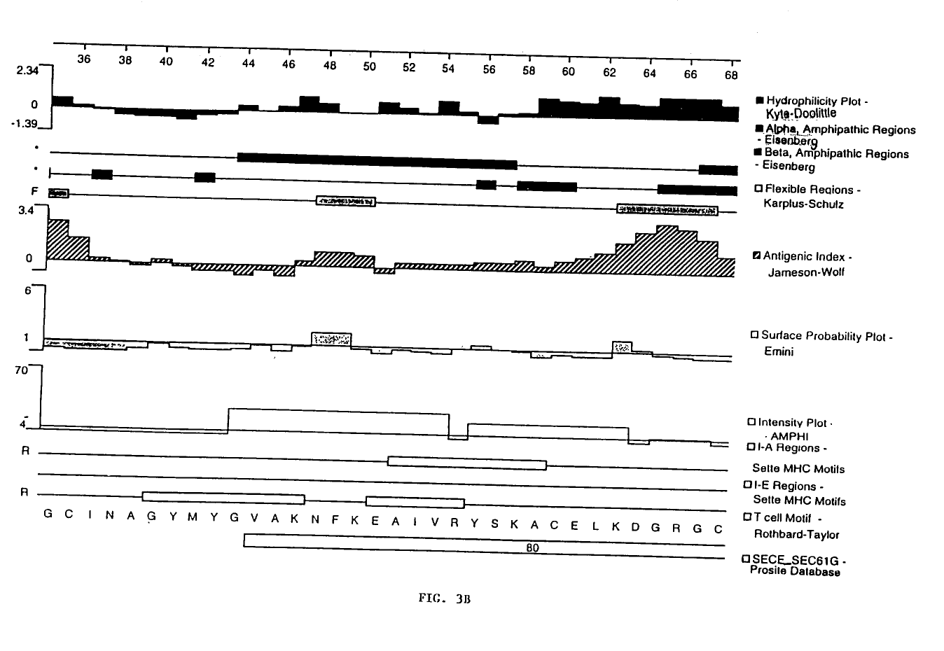

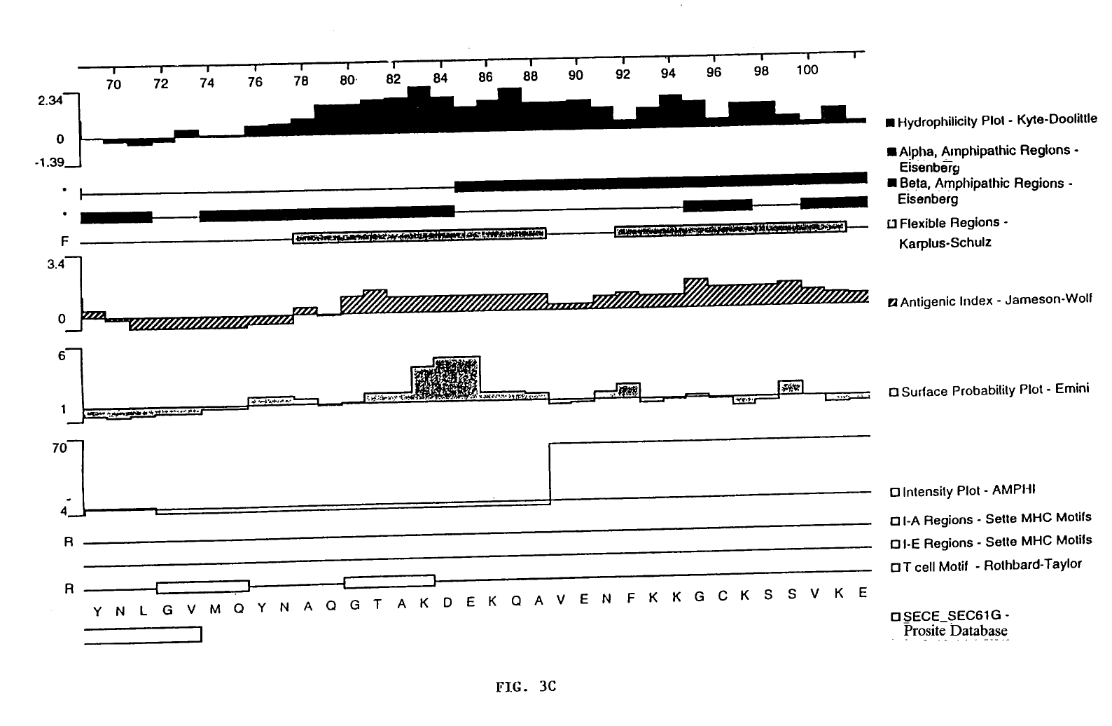

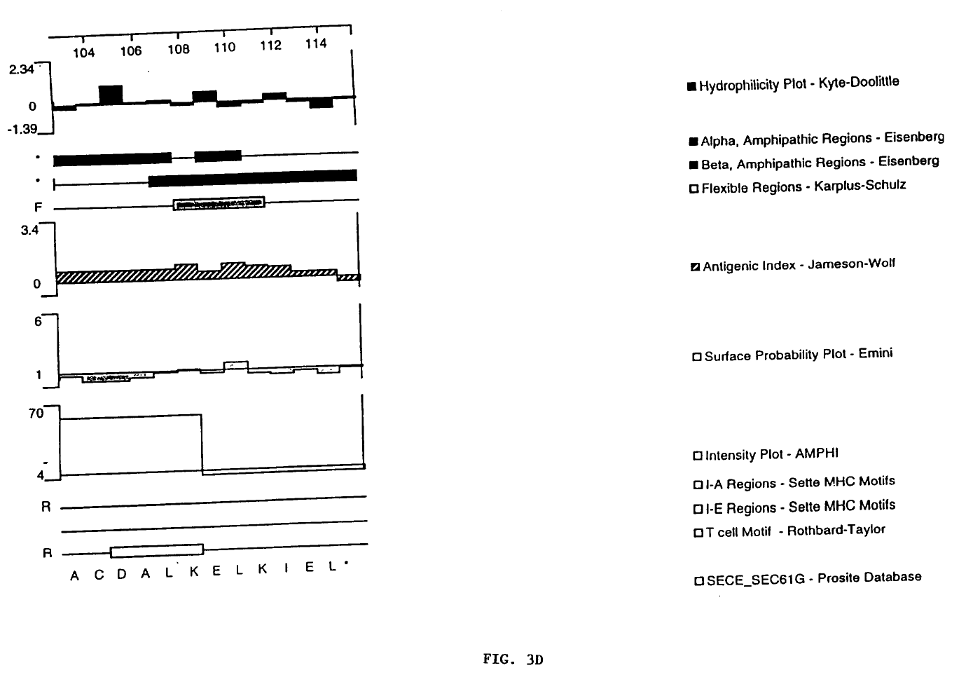

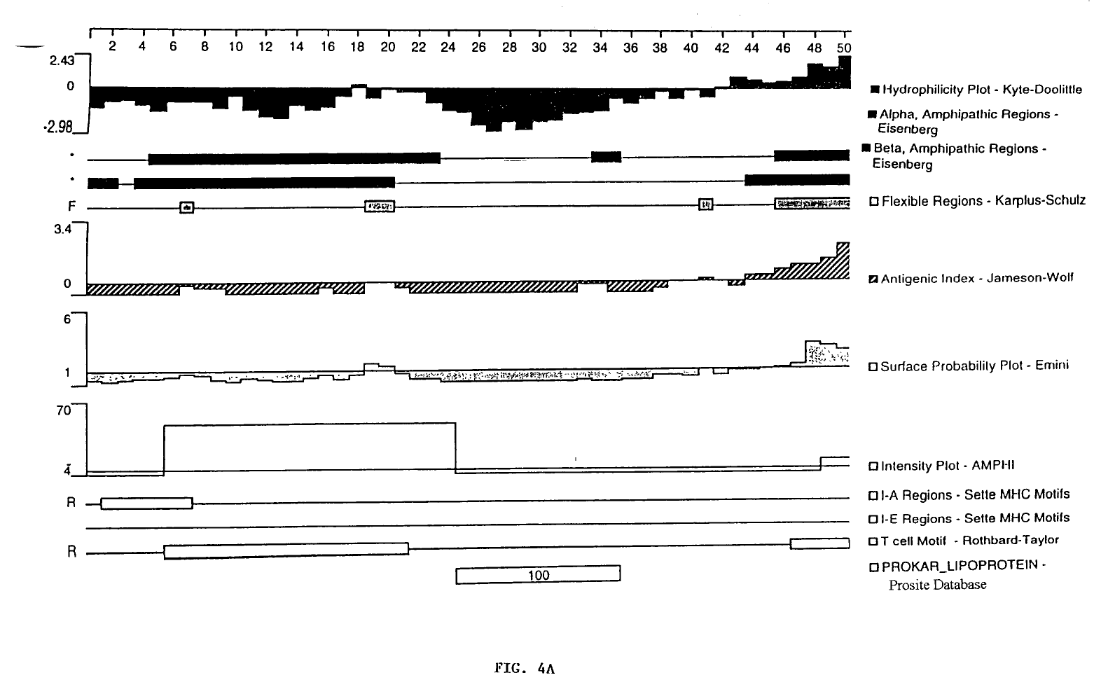

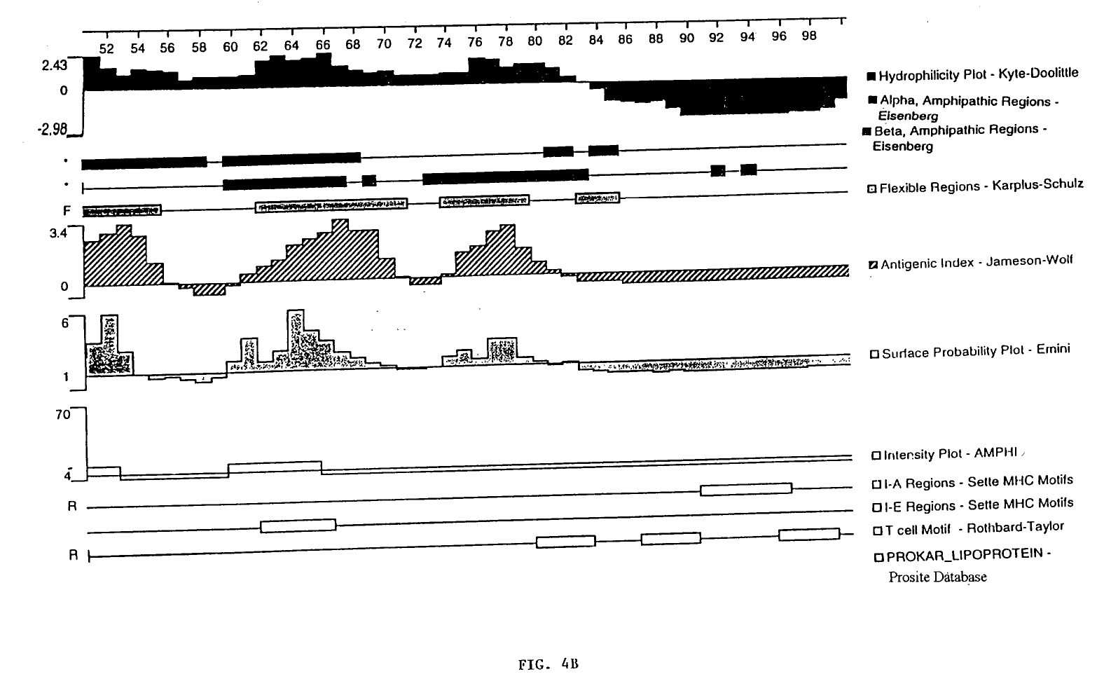

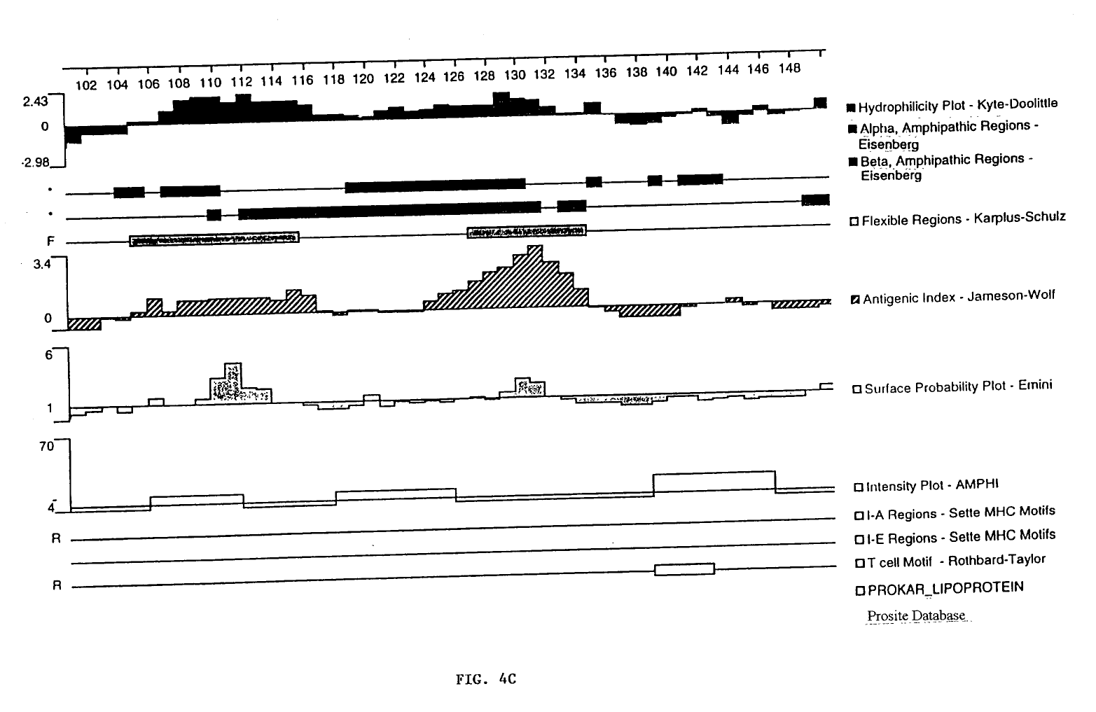

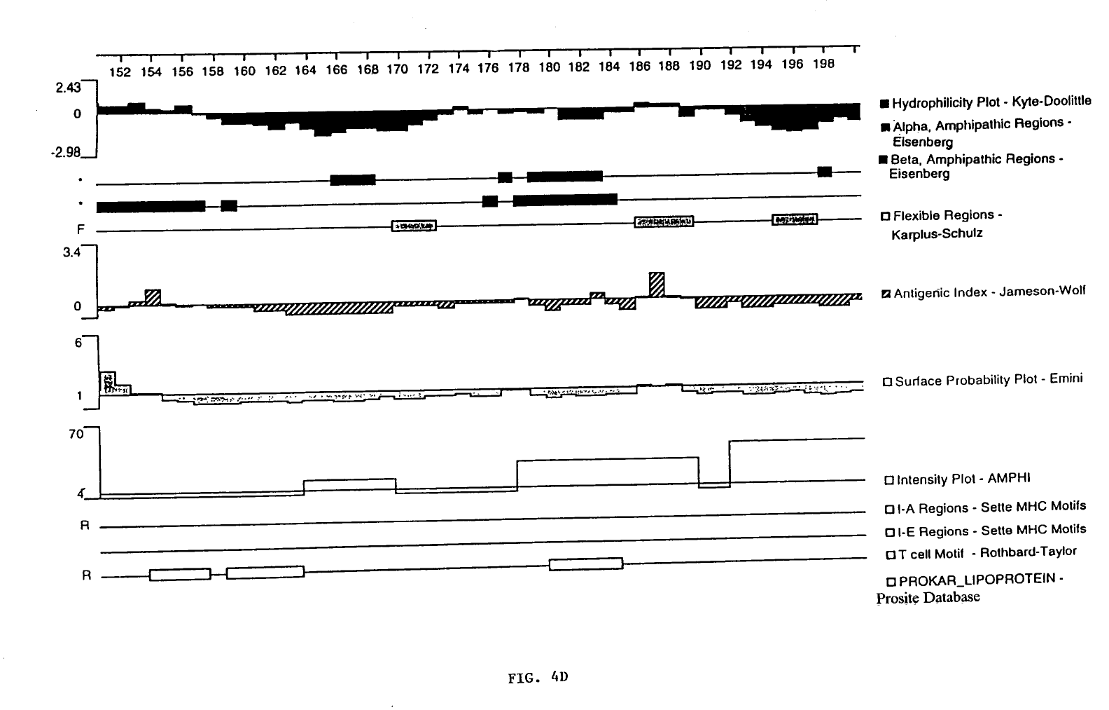

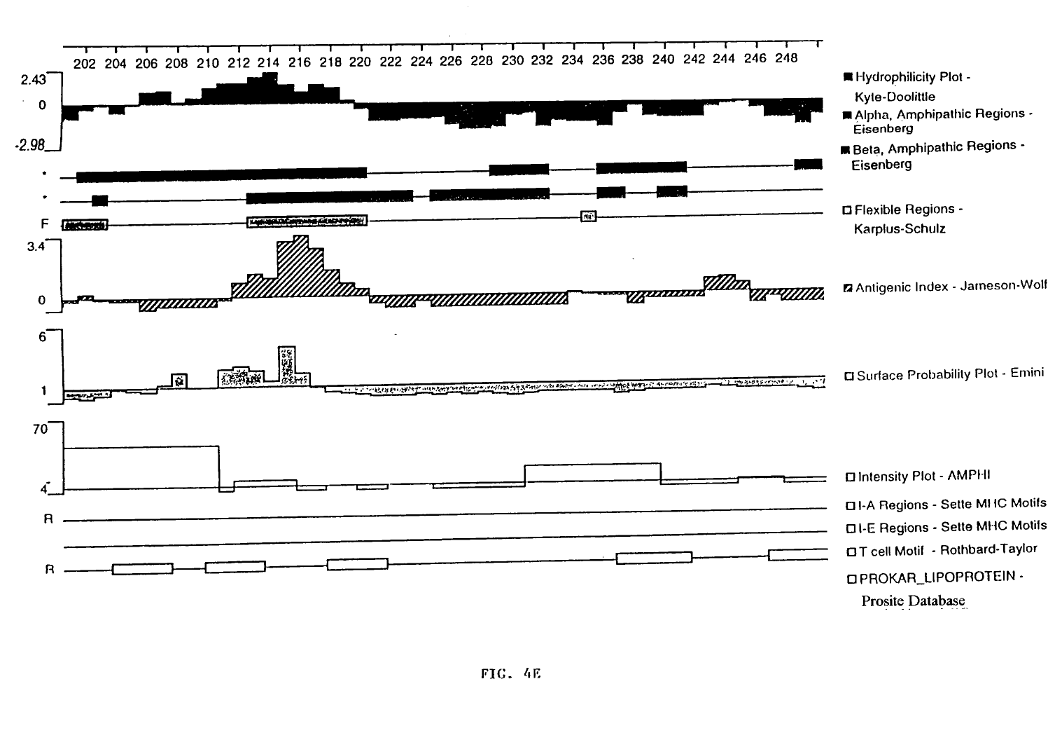

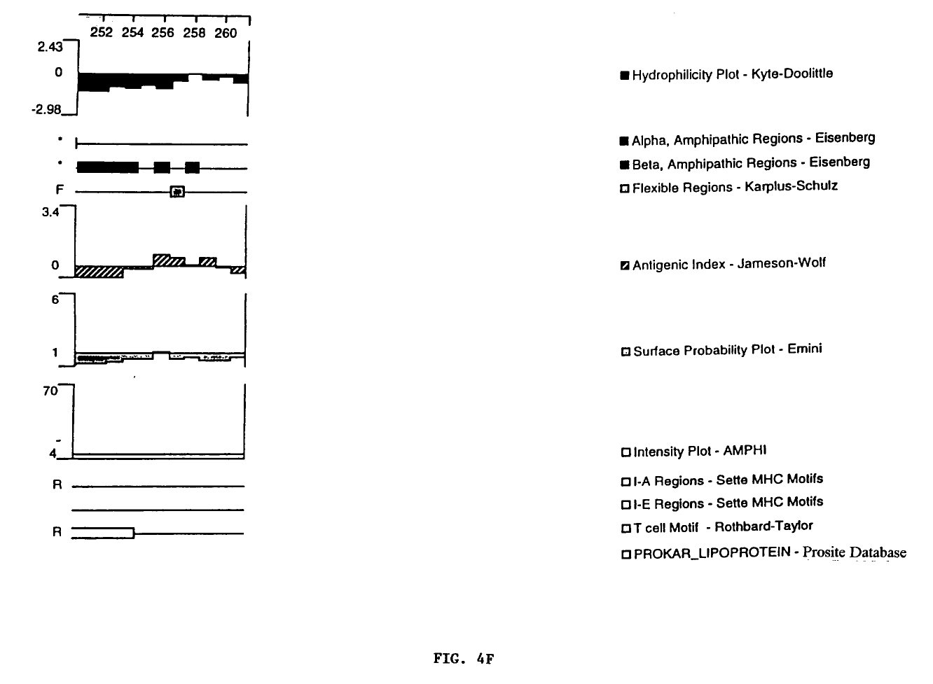

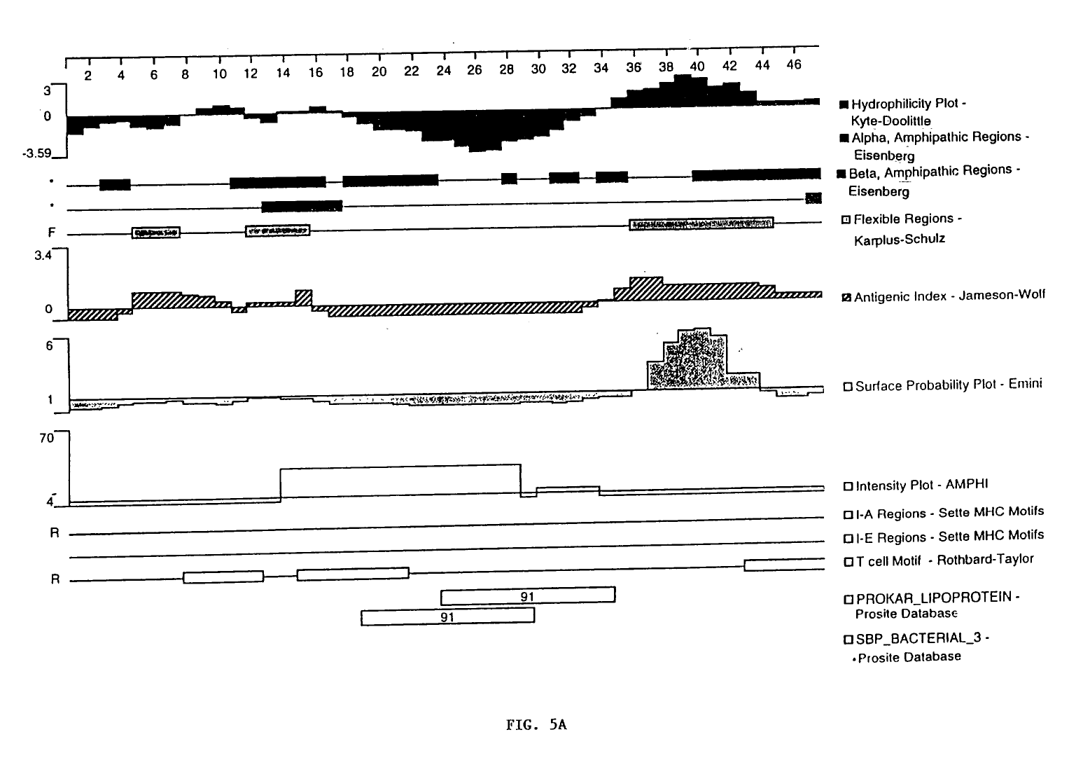

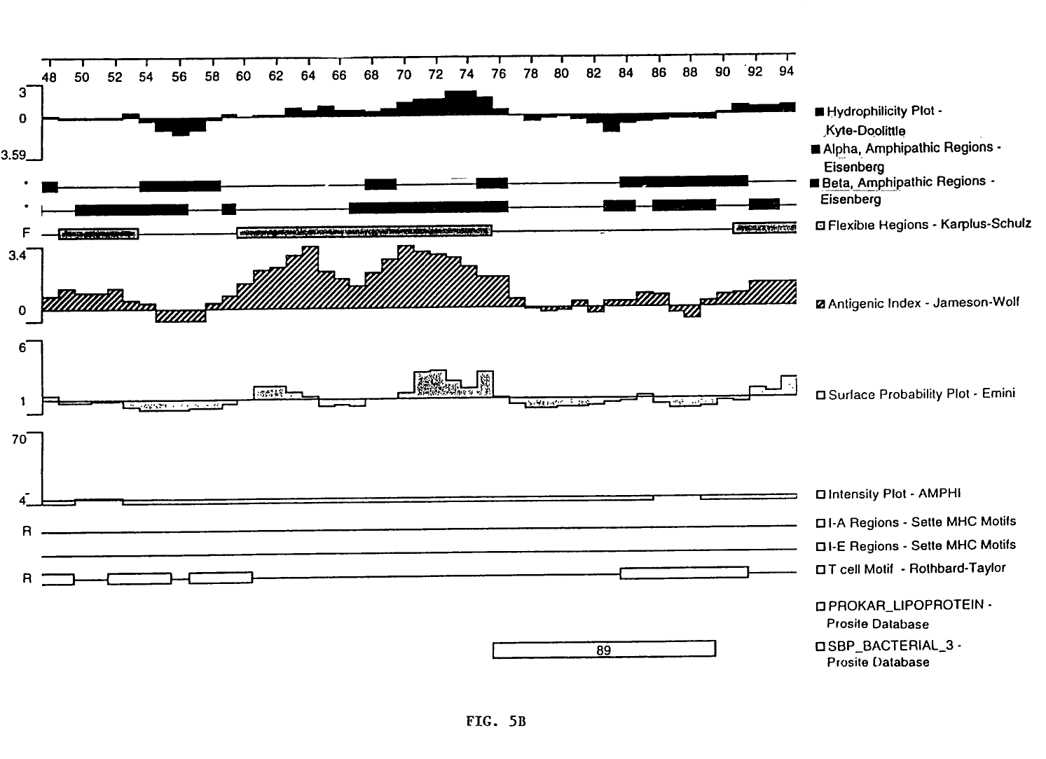

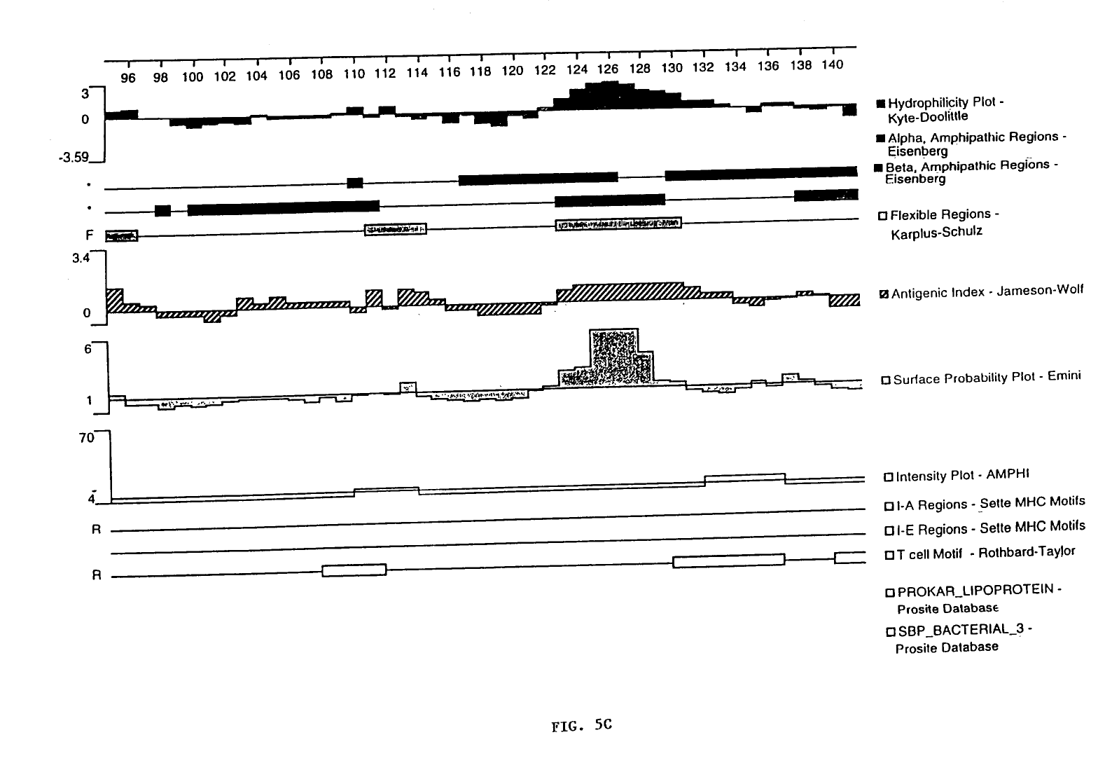

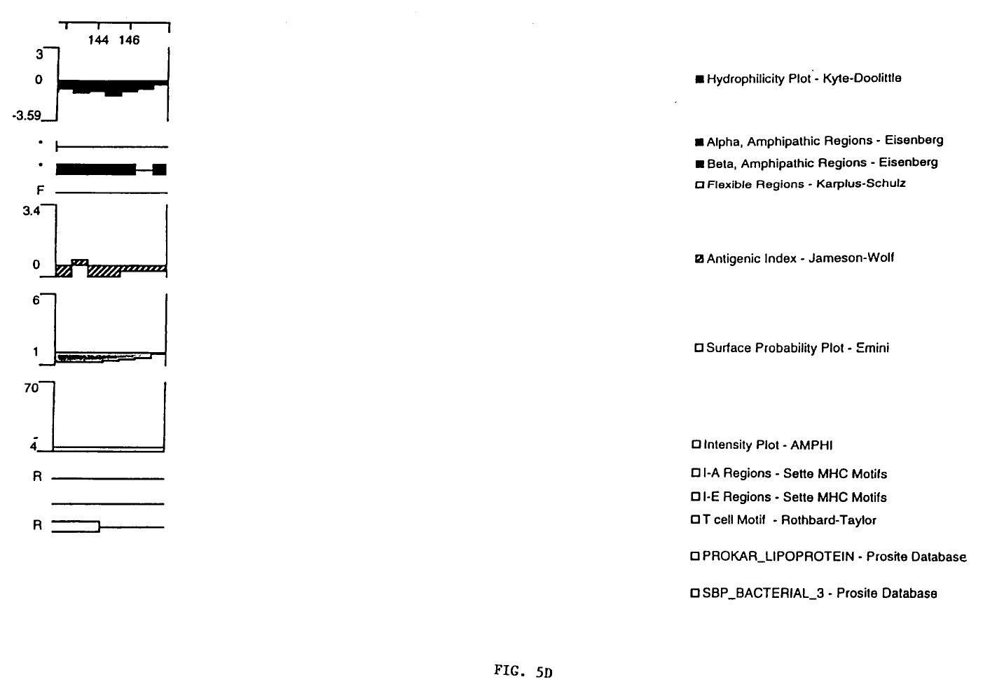

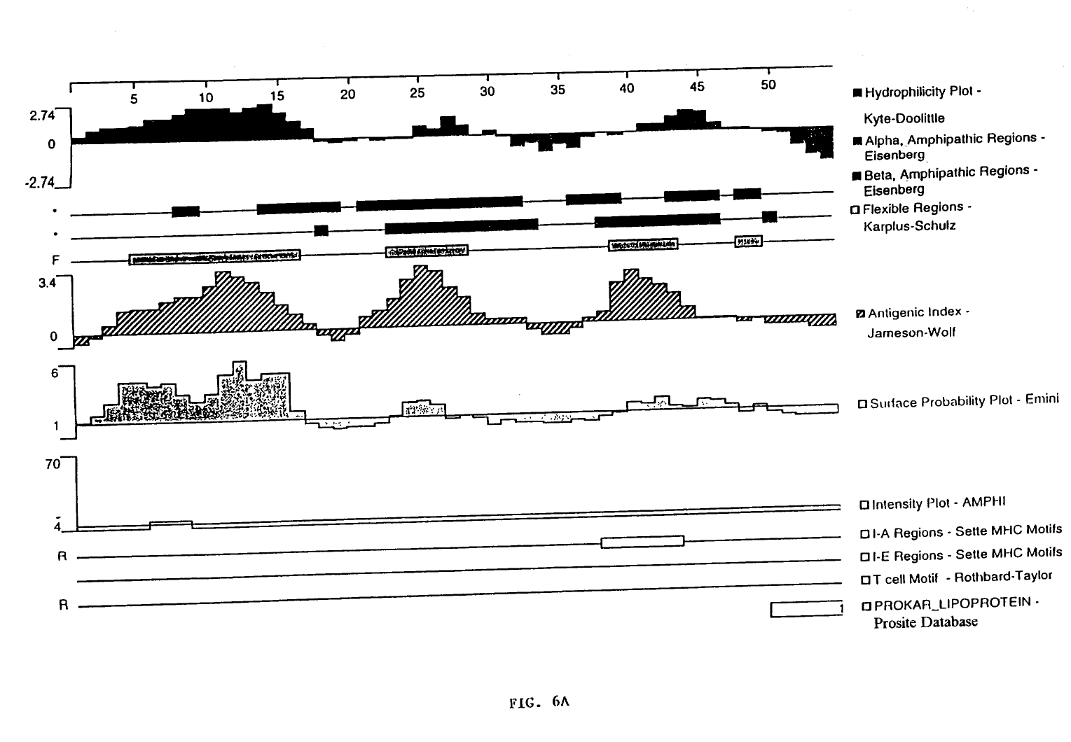

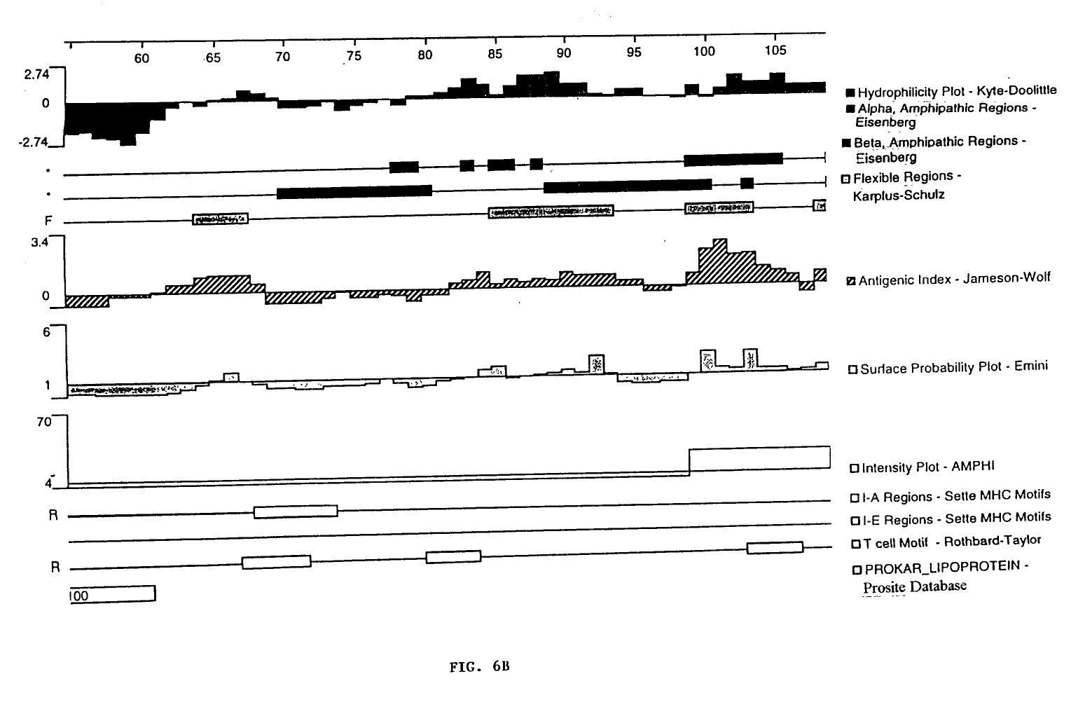

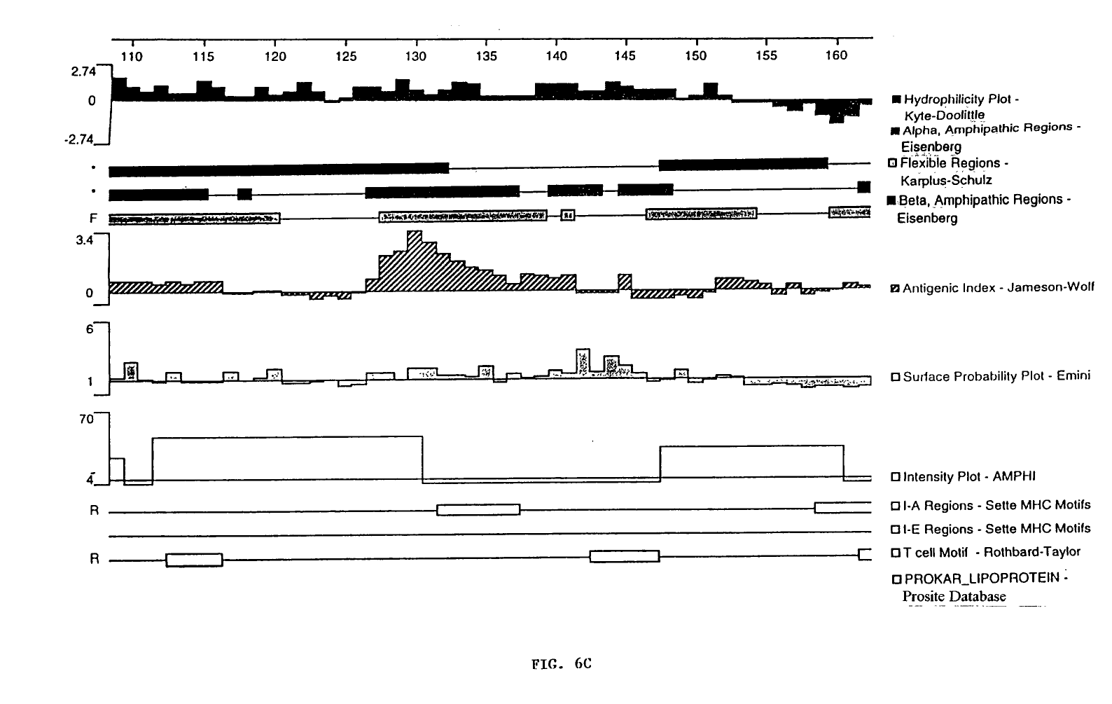

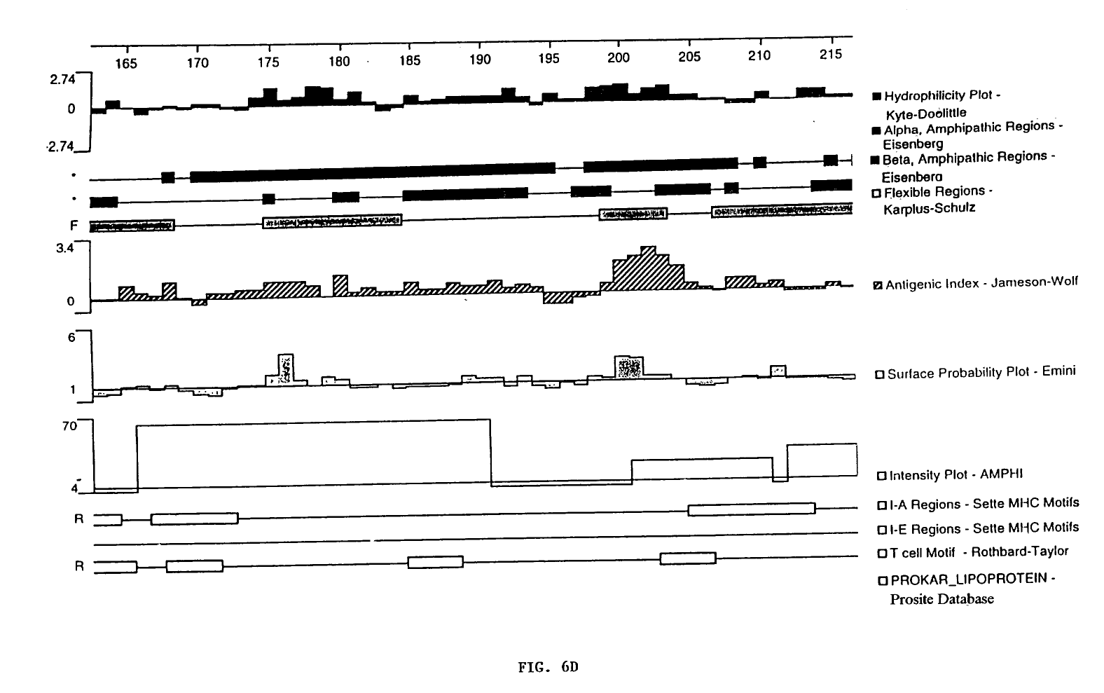

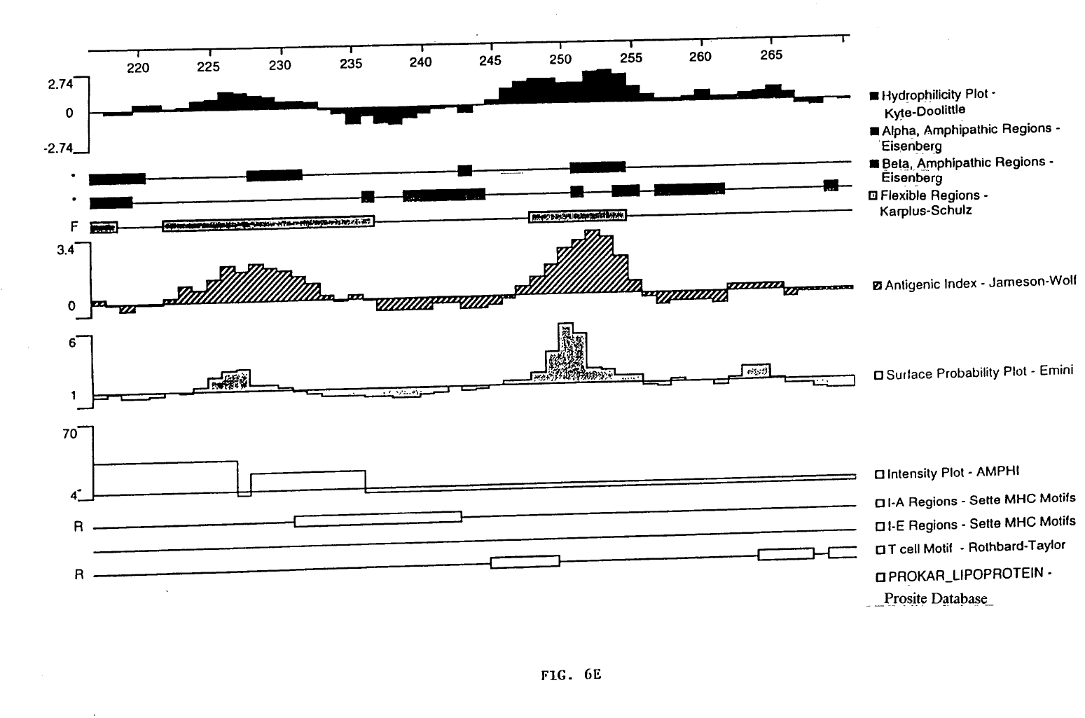

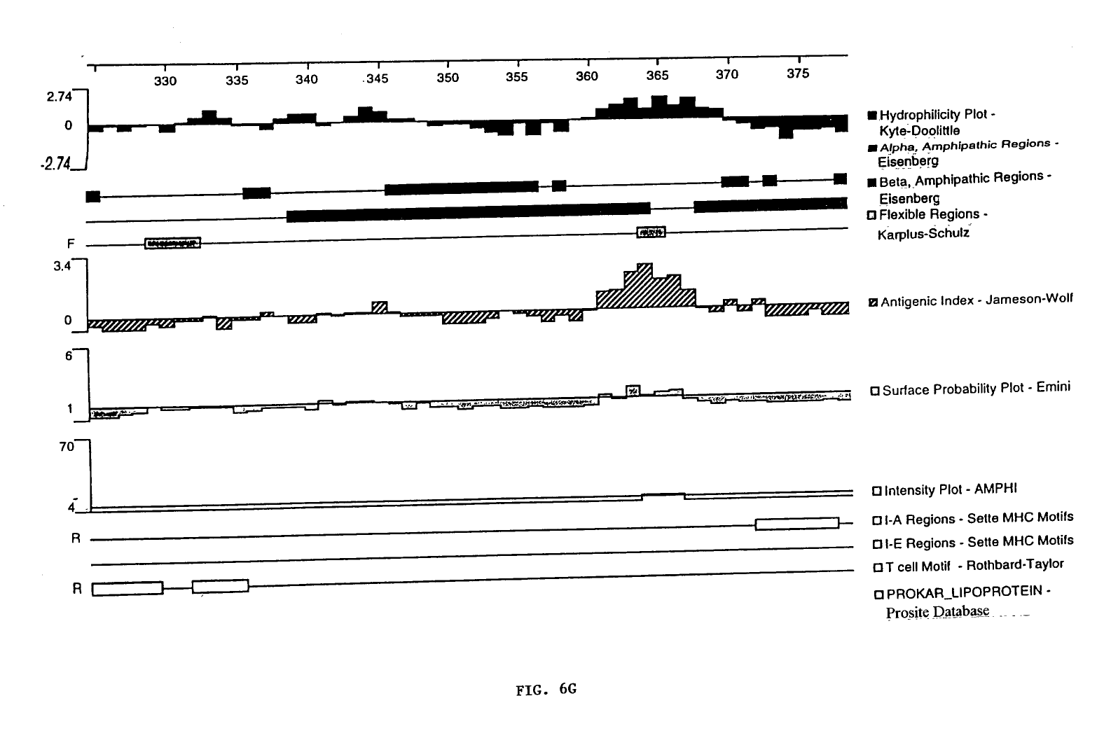

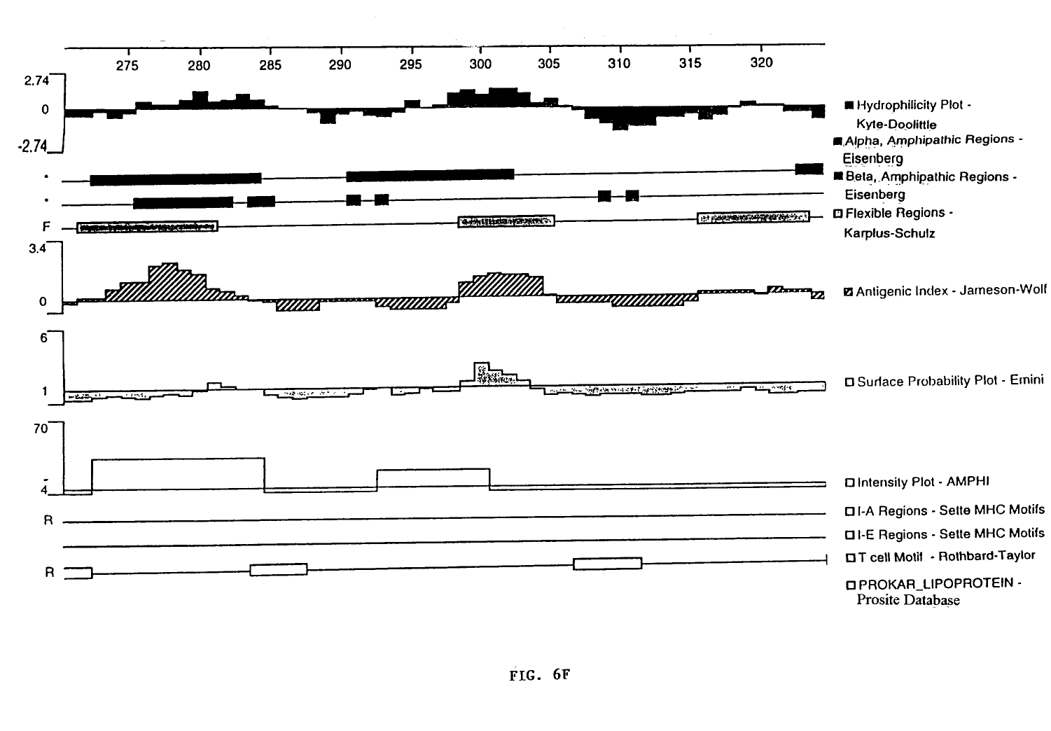

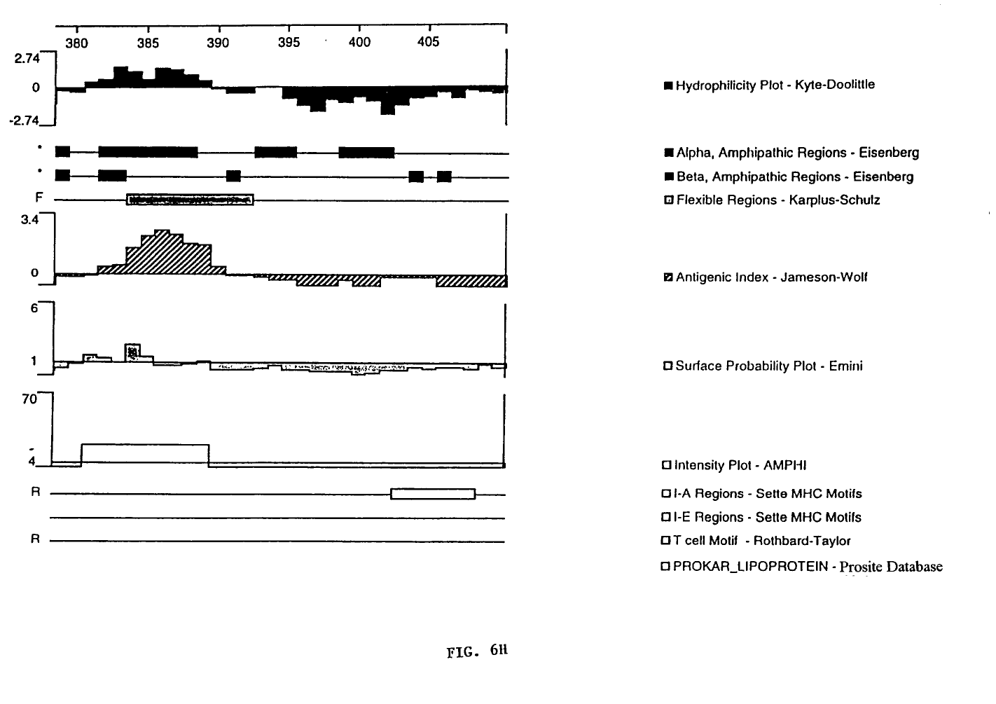

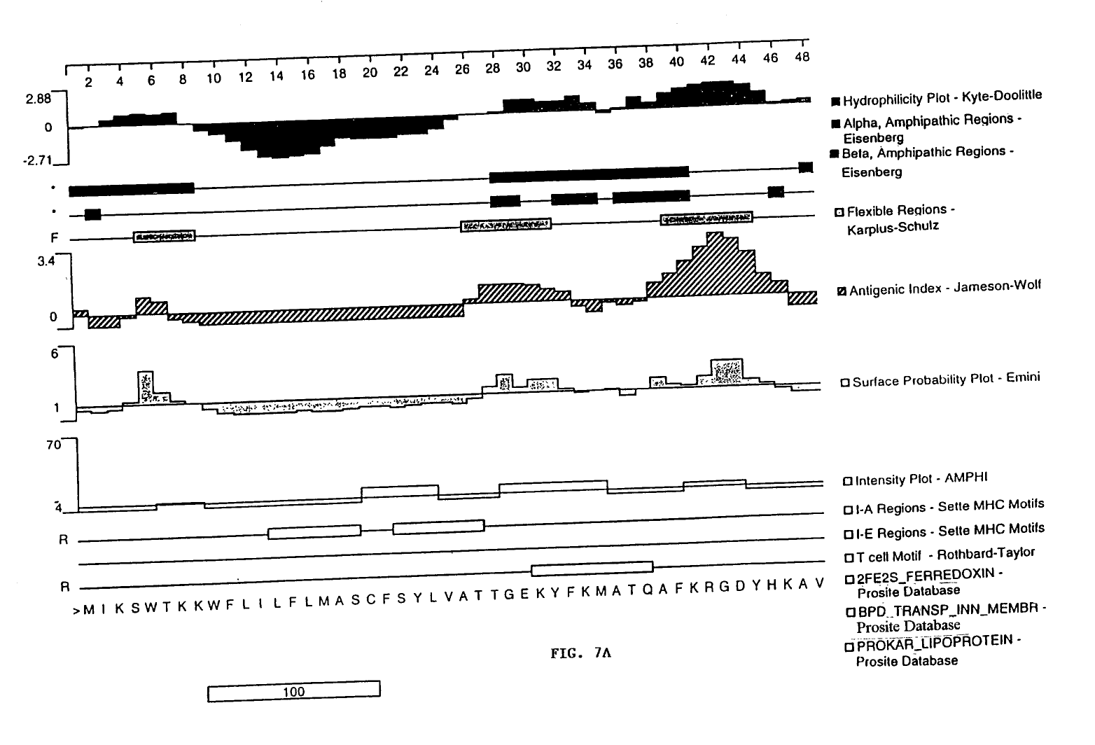

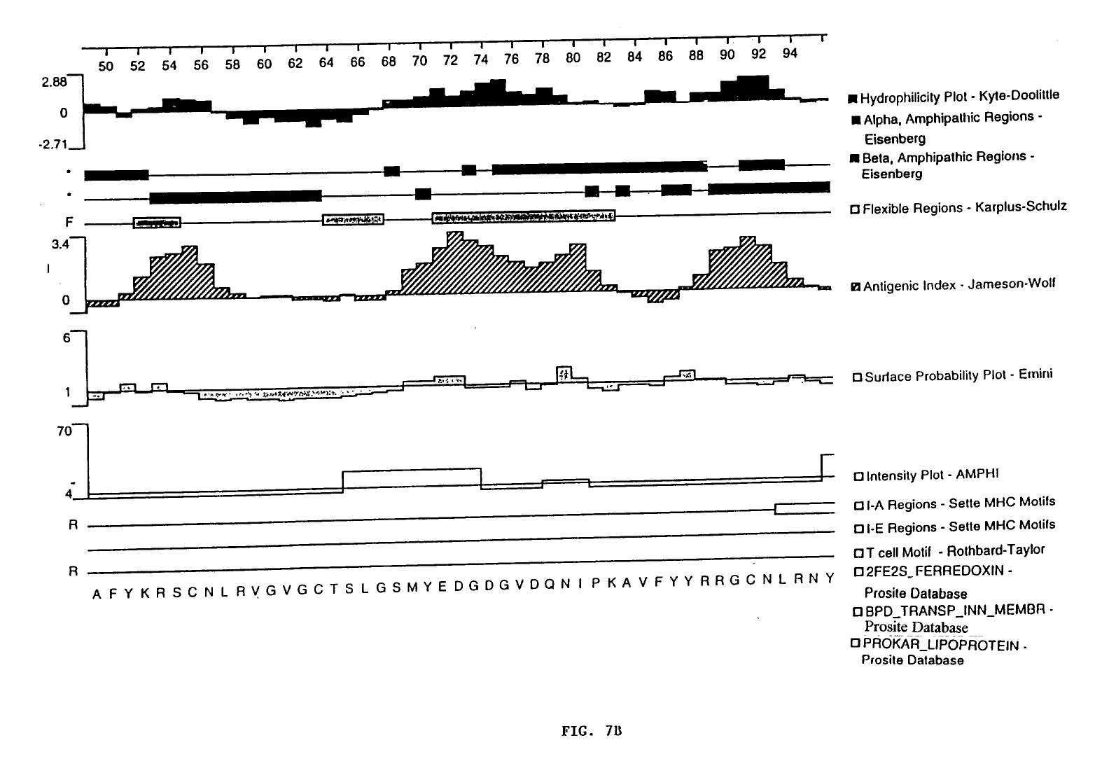

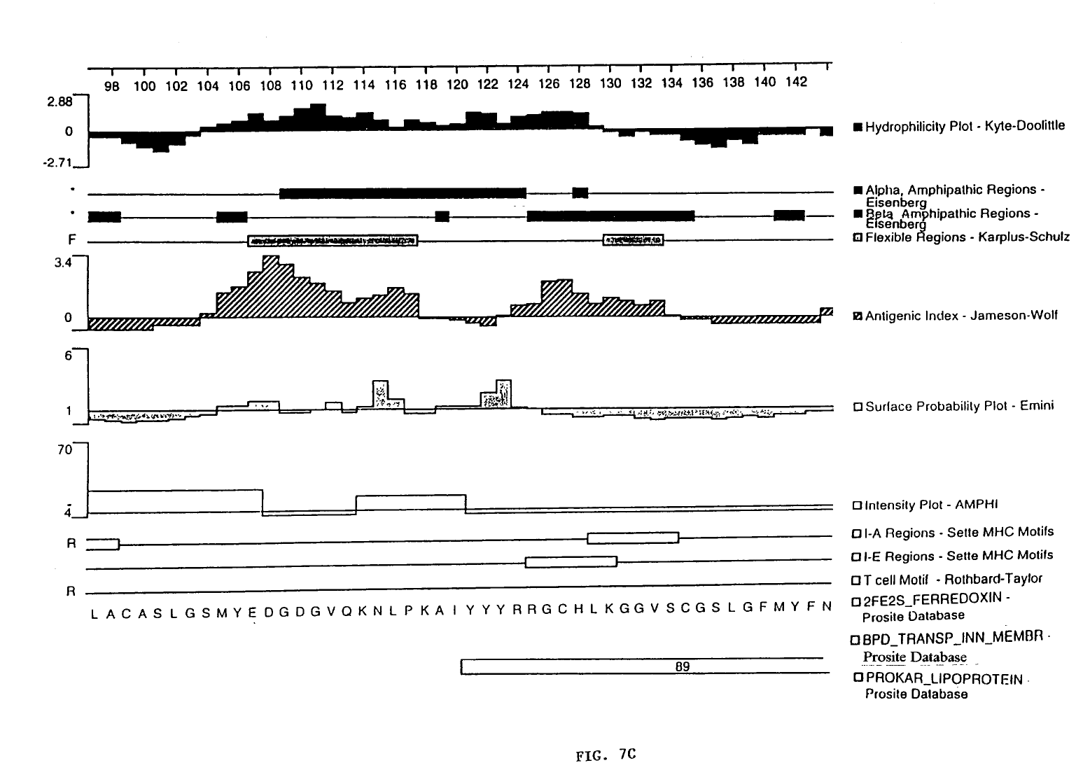

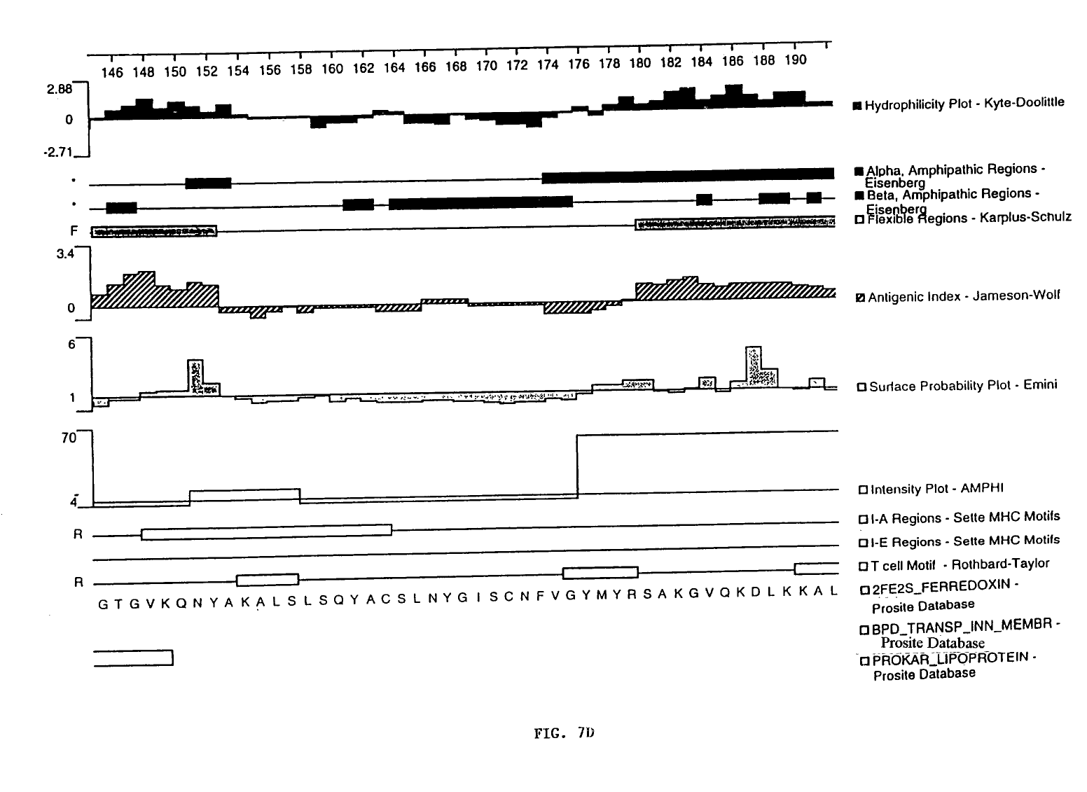

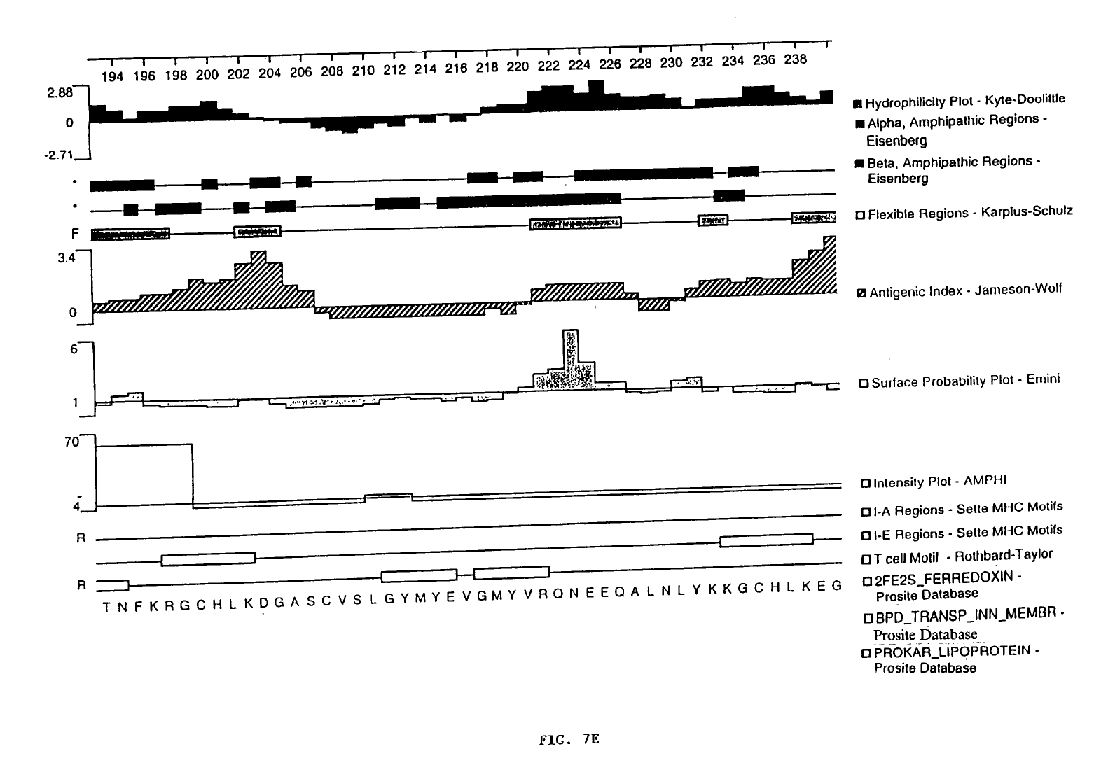

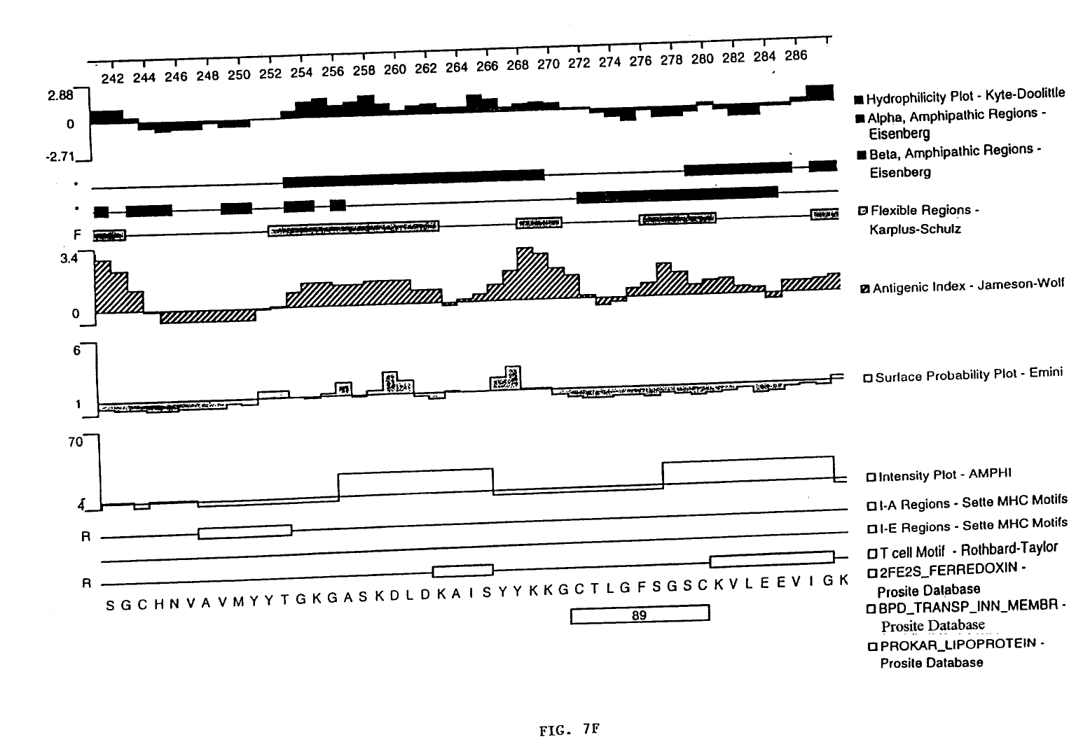

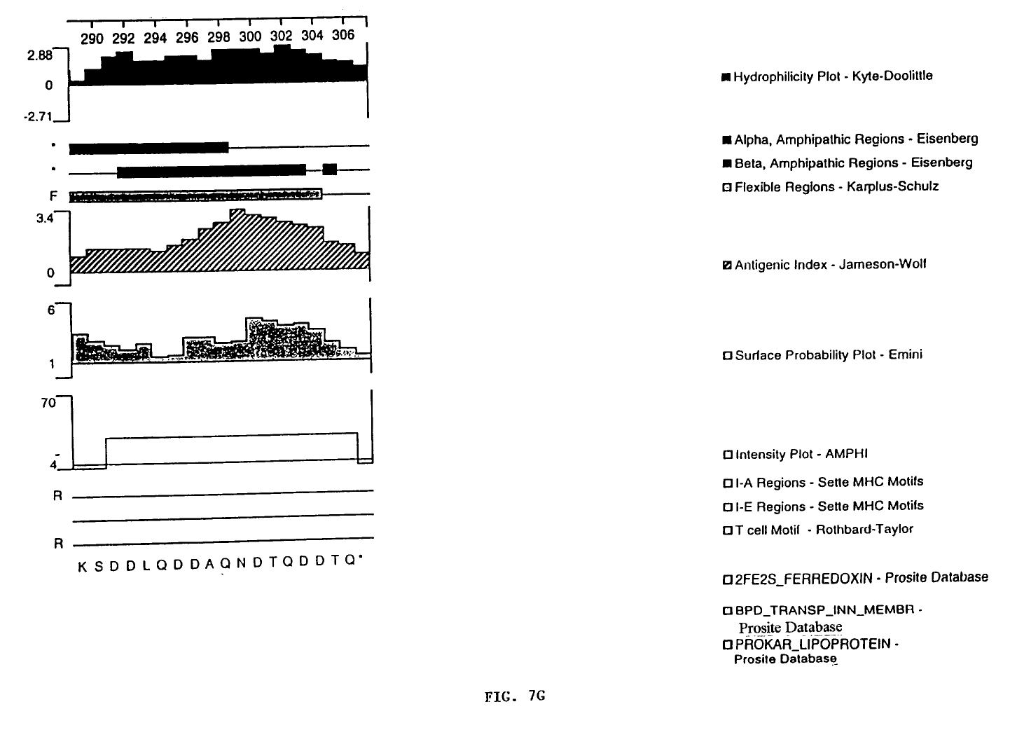

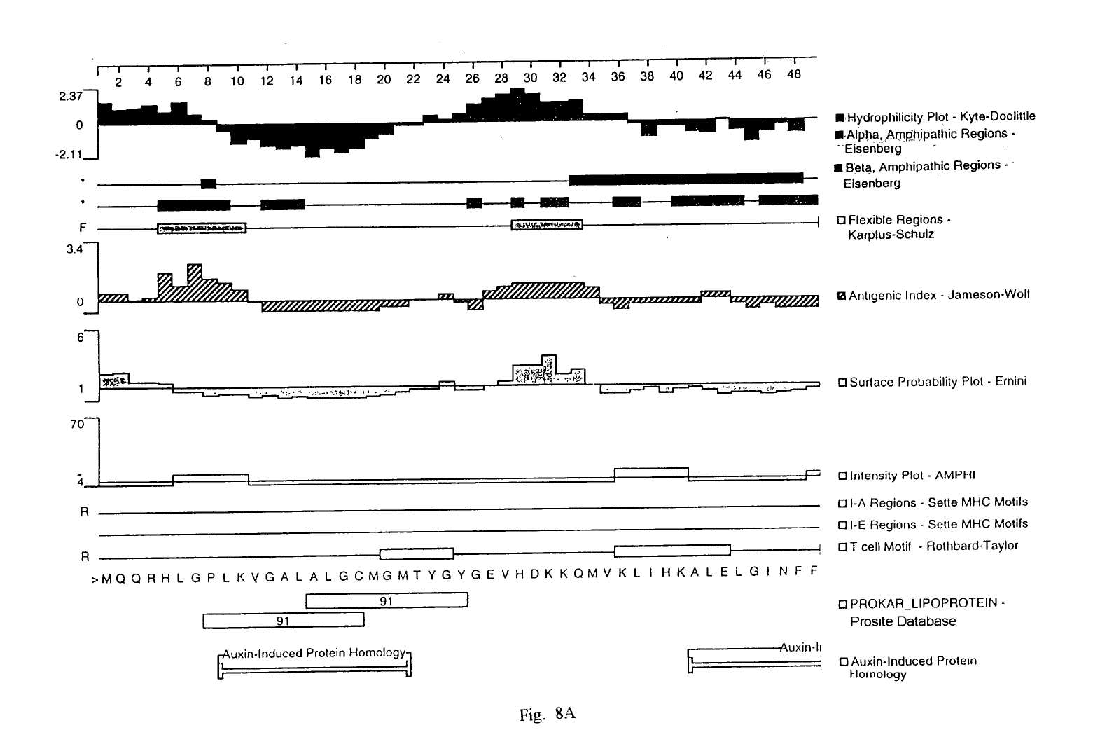

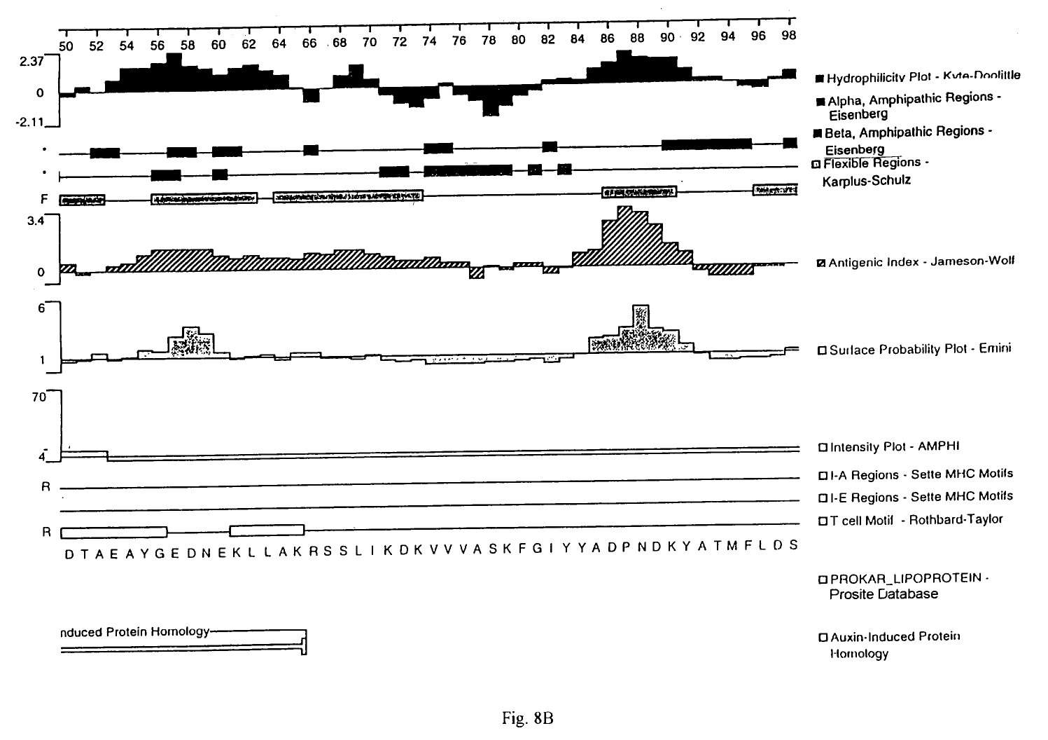

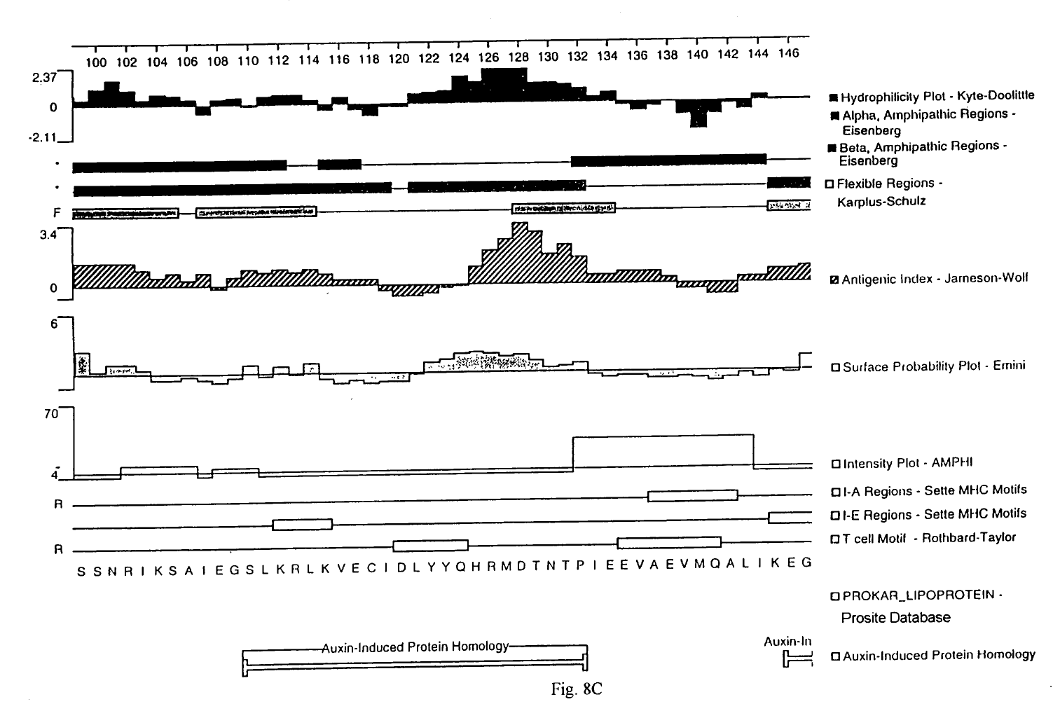

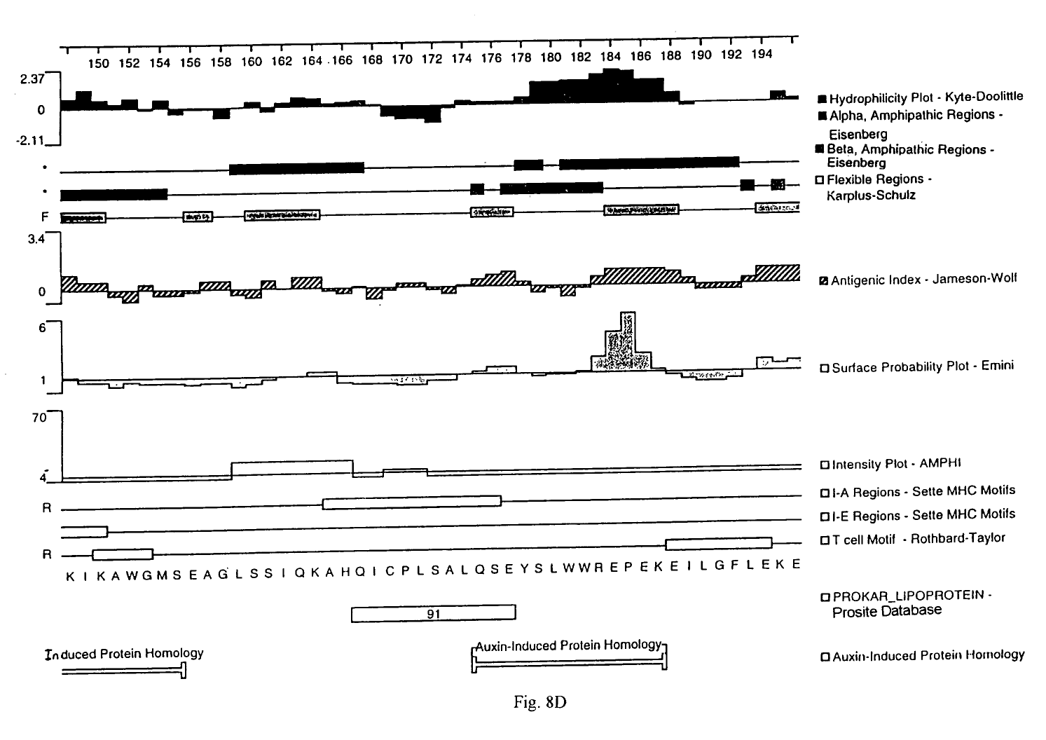

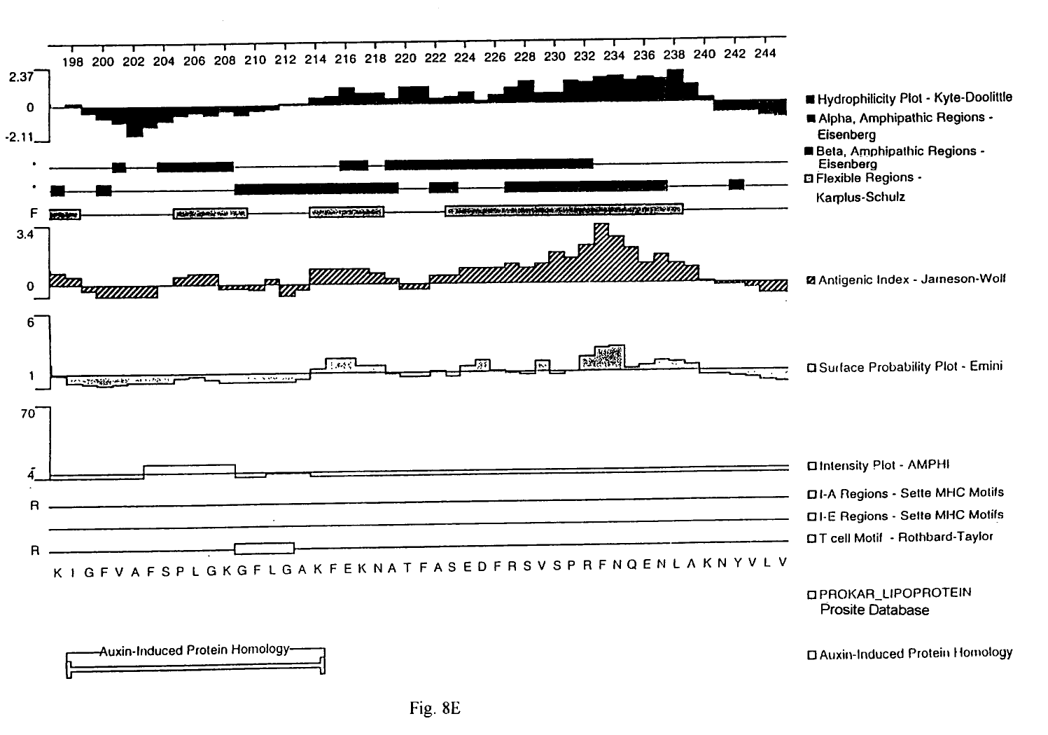

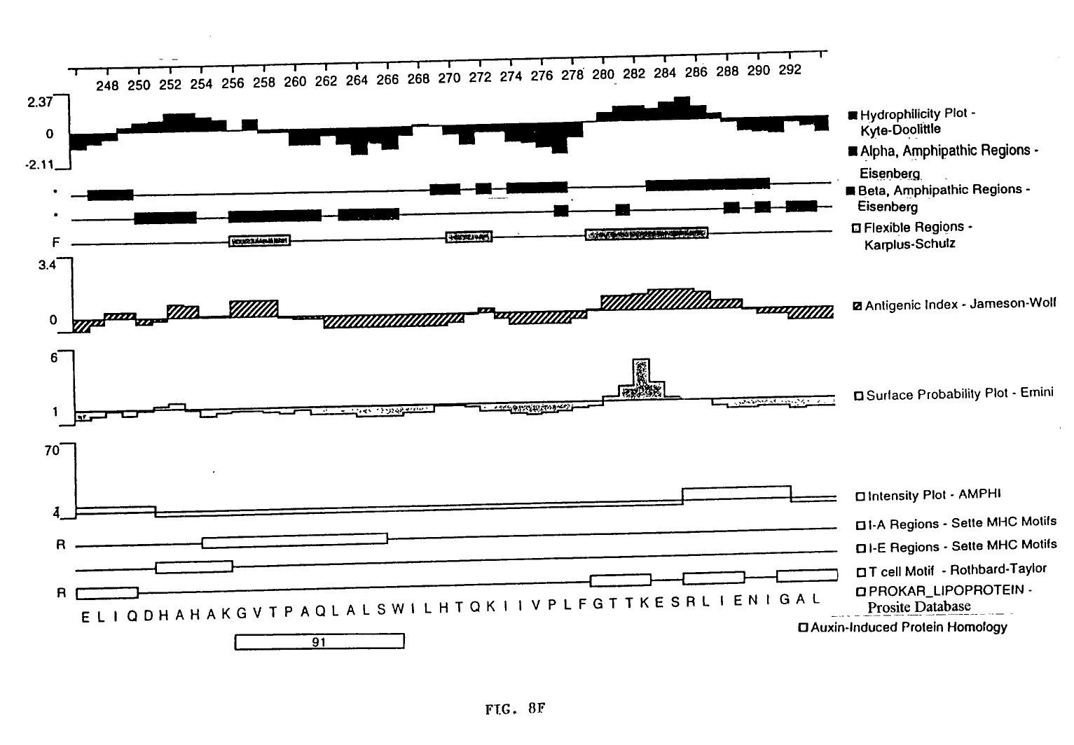

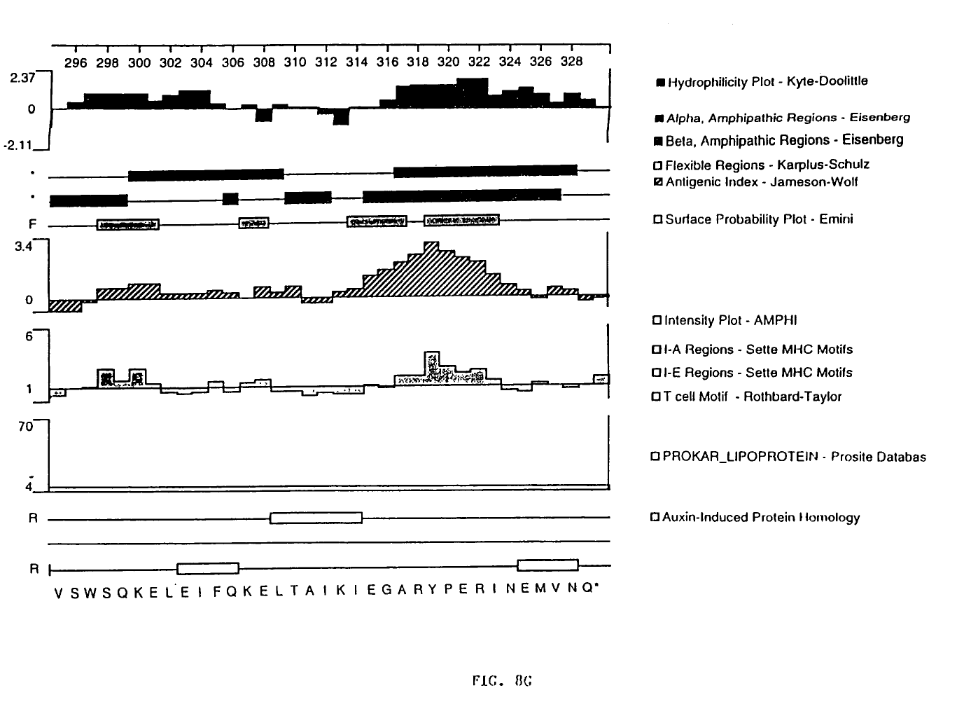

Computer-assisted analysis of some polypeptides of the invention is illustrated in FIGS. 2 to 8, which are graphs showing some of the physical properties of polypeptides HPO76 (SEQ ID NO:36), HPO15 (SEQ ID NO:12), HPO42 (SEQ ID NO:18), HPO50 (SEQ ID NO:22), HPO54 (SEQ ID NO:24), HPO57 (SEQ ID NO:26), and HPO64 (SEQ ID NO:30). The graphs were prepared using the Laser Gene Program from DNA Star, and include, e.g., hydrophilicity, antigenic index, and intensity index plots. Also included in the graphs are spots showing homologies with known protein motifs, such as the T-cell recognition motif and the major histocompatibility complex (MHC) IA and IE regions of mice. One skilled in the art can readily use the information provided in such plots to select peptide fragments for use as vaccine antigens. For example, fragments spanning regions of the plots in which the antigenic index is relatively high can be selected. One can also select fragments spanning regions in which both the antigenic index and the intensity plots are relatively high. Fragments containing conserved sequences, particularly hydrophilic conserved sequences, can also be selected.

Polypeptide fragments and polypeptides having large internal deletions can be used for revealing epitopes that are otherwise masked in the parent polypeptide and that may be of importance for inducing a protective T cell-dependent immune response. Deletions can also remove immunodominant regions of high variability among strains.

It is an accepted practice in the field of immunology to use fragments and variants of protein immunogens as vaccines, as all that is required to induce an immune response to a protein is a small (e.g., 8 to 10 amino acid) immunogenic region of the protein. This has been done for a number of vaccines against pathogens other than Helicobacter. For example, short synthetic peptides corresponding to surface-exposed antigens of pathogens such as murine mammary tumor virus (peptide containing 11 amino acids; Dion et al., Virology 179:474-477, 1990), Semliki Forest virus (peptide containing 16 amino acids; Snijders et al., J. Gen. Virol. 72:557-565, 1991), and canine parvovirus (2 overlapping peptides, each containing 15 amino acids; Langeveld et al., Vaccine 12(15): 1473-1480, 1994) have been shown to be effective vaccine antigens against their respective pathogens.

Polynucleotides encoding polypeptide fragments and polypeptides having large internal deletions can be constructed using standard methods (see, e.g., Ausubel et al., Current Protocols in Molecular Biology, John Wiley & Sons Inc., 1994), for example, by PCR, including inverse PCR, by restriction enzyme treatment of the cloned DNA molecules, or by the method of Kunkel et al. (Proc. Natl. Acad. Sci. U.S.A. 82:448, 1985; biological material available at Stratagene).

A polypeptide derivative can also be produced as a fusion polypeptide that contains a polypeptide or a polypeptide derivative of the invention fused, e.g., at the N- or C-terminal end, to any other polypeptide (hereinafter referred to as a peptide tail). Such a product can be easily obtained by translation of a genetic fusion, i.e., a hybrid gene. Vectors for expressing fusion polypeptides are commercially available, such as the pMal-c2 or pMal-p2 systems of New England Biolabs, in which the peptide tail is a maltose binding protein, the glutathione-S-transferase system of Pharmacia, or the His-Tag system available from Novagen. These and other expression systems provide convenient means for further purification of polypeptides and derivatives of the invention.

Another particular example of fusion polypeptides included in invention includes a polypeptide or polypeptide derivative of the invention fused to a polypeptide having adjuvant activity, such as, e.g., subunit B of either cholera toxin or E. coli heat-labile toxin. Several possibilities are can be used for achieving fusion. First, the polypeptide of the invention can be fused to the N-, or preferably, to the C-terminal end of the polypeptide having adjuvant activity. Second, a polypeptide fragment of the invention can be fused within the amino acid sequence of the polypeptide having adjuvant activity.

As stated above, the polynucleotides of the invention encode Helicobacter polypeptides in precursor or mature form. They can also encode hybrid precursors containing heterologous signal peptides, which can mature into polypeptides of the invention. By “heterologous signal peptide” is meant a signal peptide that is not found in the naturally-occurring precursor of a polypeptide of the invention.

A polynucleotide of the invention, having a homologous coding sequence, hybridizes, preferably under stringent conditions, to a polynucleotide having a sequence as shown in SEQ ID NOs:1 to 47 (odd numbers) or to an insert of a deposited DNA molecule (see below, e.g., Example 2). Hybridization procedures are, e.g., described in Ausubel et al., supra; Silhavy et al. (Experiments with Gene Fusions, Cold Spring Harbor Laboratory Press, 1984); Davis et al. (A Manual for Genetic Engineering: Advanced Bacterial Genetics, Cold Spring Harbor Laboratory Press, 1980). Important parameters that can be considered for optimizing hybridization conditions are reflected in a formula that allows calculation of a critical value, the melting temperature above which two complementary DNA strands separate from each other (Casey et al., Nucl. Acid Res. 4:1539, 1997). This formula is as follows: Tm=81.5+0.5×(% G+C)+1.6 log (positive ion concentration) −0.6×(% formamide). Under appropriate stringency conditions, hybridization temperature (Th) is approximately 20 to 40° C., 20 to 25° C., or, preferably 30 to 40° C. below the calculated Tm. Those skilled in the art will understand that optimal temperature and salt conditions can be readily determined empirically in preliminary experiments using conventional procedures.

For example, stringent conditions can be achieved, both for pre-hybridizing and hybridizing incubations, (i) within 4-16 hours at 42° C., in 6× SSC containing 50% formamide or (ii) within 4-16 hours at 65° C. in an aqueous 6× SSC solution (1 M NaCl, 0.1 M sodium citrate (pH 7.0)).

For polynucleotides containing 30 to 600 nucleotides, the above formula is used and then is corrected by subtracting (600/polynucleotide size in base pairs). Stringency conditions are defined by a Th that is 5 to 10° C. below Tm.

Hybridization conditions with oligonucleotides shorter than 20-30 bases do not exactly follow the rules set forth above. In such cases, the formula for calculating the Tm is as follows: Tm=4×(G+C)+2(A+T). For example, an 18 nucleotide fragment of 50% G+C would have an approximate Tm of 54° C.

Plasmids containing nucleic acids encoding HPO101, HPO104, HPO116, HPO121, HPO132, HPO15, HPO18, HPO38, HPO42, HPO45, HPO50, HPO54, HPO57, HPO58, HPO64, HPO70, HPO71, HPO76, HPO7, HPO80, HPO87, HPO95, HPO98, and HPO9 were deposited in E. coli strain DH5α under the Budapest Treaty, with the American Type Culture Collection (ATCC; Rockville, Md.) on Oct. 9, 1996 and were designated with accession numbers listed below in Example 2. These plasmids were derived from pMin2 by insertion of a genomic DNA BglII-ClaI fragment from H. pylori strain P1 or P12 into the vector. Each of the inserts is disrupted by the presence of transposon TnMax9 (Kahrs et al., Gene 167:53, 1995). The locations of insertion of the transposon in each of the deposited clones (see below) are between the nucleotides indicated in parentheses after the name of each clone, as follows: HPO101 (497-498), HPO104 (428-429), HPO116 (433-444), HPO121 (463-464), HPO132 (408-409), HPO18 (226-227), HPO38 (347-348), HPO42 (372-373), HPO45 (299-300), HPO50 (29-293), HPO54 (351-352), HPO57 (266-267), HPO58 (434-435), HPO64 (224-225), HPO70 (114-115), HPO71 (274-275), HPO76 (412-413), HPO7 (349-350), HPO80 (105-106), HPO87 (26-27), HPO95 (64-65), HPO98 (43-44), and HPO9 (346-347). As is discussed further below in Example 2, DNA molecules lacking the transposon can be amplified from the plasmids using standard PCR techniques, including inverse and recombinant PCR (see, e.g., PCR protocols: A Guide to Methods and Applications (1990) Innis et al., Eds., Academic Press), so that the full-length H. pylori insert is reconstituted.

A polynucleotide molecule of the invention, containing RNA, DNA, or modifications or combinations thereof, can have various applications. For example, a DNA molecule can be used (i) in a process for producing the encoded polypeptide in a recombinant host system, (ii) in the construction of vaccine vectors such as pox viruses, which are further used in methods and compositions for preventing and/or treating Helicobacter infection, (iii) as a vaccine agent (as well as an RNA molecule), in a naked form or formulated with a delivery vehicle and, (iv) in the construction of attenuated Helicobacter strains that can over-express a polynucleotide of the invention or express it in a non-toxic, mutated form.

According to a second aspect of the invention, there is therefore provided (i) an expression cassette containing a DNA molecule of the invention placed under the control of the elements required for expression, in particular under the control of an appropriate promoter; (ii) an expression vector containing an expression cassette of the invention; (iii) a procaryotic or eucaryotic cell transformed or transfected with an expression cassette and/or vector of the invention, as well as (iv) a process for producing a polypeptide or polypeptide derivative encoded by a polynucleotide of the invention, which involves culturing a procaryotic or eucaryotic cell transformed or transfected with an expression cassette and/or vector of the invention, under conditions that allow expression of the DNA molecule of the invention and, recovering the encoded polypeptide or polypeptide derivative from the cell culture.

A recombinant expression system can be selected from procaryotic and eucaryotic hosts. Eucaryotic hosts include yeast cells (e.g., Saccharomyces cerevisiae or Pichia Pastoris), mammalian cells (e.g., COS1, NIH3T3, or JEG3 cells), arthropods cells (e.g., Spodoptera frugiperda (SF9) cells), and plant cells. Preferably, a procaryotic host such as E. coli is used. Bacterial and eucaryotic cells are available from a number of different sources to those skilled in the art, e.g., the American Type Culture Collection (ATCC; Rockville, Md.).

The choice of the expression system depends on the features desired for the expressed polypeptide. For example, it may be useful to produce a polypeptide of the invention in a particular lipidated form or any other form.

The choice of the expression cassette will depend on the host system selected as well as the features desired for the expressed polypeptide. Typically, an expression cassette includes a promoter that is functional in the selected host system and can be constitutive or inducible; a ribosome binding site; a start codon (ATG) if necessary, a region encoding a signal peptide, e.g., a lipidation signal peptide; a DNA molecule of the invention; a stop codon; and optionally a 3′ terminal region (translation and/or transcription terminator). The signal peptide-encoding region is adjacent to the polynucleotide of the invention and placed in proper reading frame. The signal peptide-encoding region can be homologous or heterologous to the DNA molecule encoding the mature polypeptide and can be specific to the secretion apparatus of the host used for expression. The open reading frame constituted by the DNA molecule of the invention, solely or together with the signal peptide, is placed under the control of the promoter so that transcription and translation occur in the host system. Promoters, signal peptide encoding regions are widely known and available to those skilled in the art and includes, for example, the promoter of Salmonella typhimurium (and derivatives) that is inducible by arabinose (promoter araB) and is functional in Gram-negative bacteria such as E. coli (as described in U.S. Pat. No. 5,028,530, and in Cagnon et al., Protein Engineering 4(7):843, 1991); the promoter of the gene of bacteriophage T7 encoding RNA polymerase, that is functional in a number of E. coli strains expressing T7 polymerase (described in U.S. Pat. No. 4,952,496); OspA lipidation signal peptide; and RlpB lipidation signal peptide (Takase et al., J. Bact. 169:5692, 1987).

The expression cassette is typically part of an expression vector, which is selected for its ability to replicate in the chosen expression system. Expression vectors (e.g., plasmids or viral vectors) can be chosen from those described in Pouwels et al. (Cloning Vectors: A Laboratory Manual 1985, Supp. 1987). They can be purchased from various commercial sources.

Methods for transforming/transfecting host cells with expression vectors will depend on the host system selected as described in Ausubel et al., supra.

Upon expression, a recombinant polypeptide of the invention (or a polypeptide derivative) is produced and remains in the intracellular compartment, is secreted/excreted in the extracellular medium or in the periplasmic space, or is embedded in the cellular membrane. The polypeptide can then be recovered in a substantially purified form from the cell extract or from the supernatant after centrifugation of the recombinant cell culture. Typically, the recombinant polypeptide can be purified by antibody-based affinity purification or by any other method that can be readily adapted by a person skilled in the art, such as by genetic fusion to a small affinity binding domain. Antibody-based affinity purification methods are also available for purifying a polypeptide of the invention extracted from a Helicobacter strain. Antibodies useful for purifying by immunoaffinity the polypeptides of the invention can be obtained as described below.

A polynucleotide of the invention can also be useful in the vaccine field, e.g., for achieving DNA vaccination. There are two major possibilities, either using a viral or bacterial host as gene delivery vehicle (live vaccine vector) or administering the gene in a free form, e.g., inserted into a plasmid. Therapeutic or prophylactic efficacy of a polynucleotide of the invention can be evaluated as described below.

Accordingly, in a third aspect of the invention, there is provided (i) a vaccine vector such as a pox virus, containing a DNA molecule of the invention, placed under the control of elements required for expression; (ii) a composition of matter containing a vaccine vector of the invention, together with a diluent or carrier; particularly, (iii) a pharmaceutical composition containing a therapeutically or prophylactically effective amount of a vaccine vector of the invention; (iv) a method for inducing an immune response against Helicobacter in a mammal (e.g., a human; alternatively, the method can be used in veterinary applications for treating or preventing Helicobacter infection of animals, e.g., cats or birds), which involves administering to the mammal an immunogenically effective amount of a vaccine vector of the invention to elicit an immune response, e.g., a protective or therapeutic immune response to Helicobacter; and particularly, (v) a method for preventing and/or treating a Helicobacter (e.g., H. pylori, H. felis, H. mustelae, or H. heilmanii) infection, which involves administering a prophylactic or therapeutic amount of a vaccine vector of the invention to an individual in need. Additionally, the third aspect of the invention encompasses the use of a vaccine vector of the invention in the preparation of a medicament for preventing and/or treating Helicobacter infection.

A vaccine vector of the invention can express one or several polypeptides or derivatives of the invention, as well as at least one additional Helicobacter antigen such as a urease apoenzyme or a subunit, fragment, homolog, mutant, or derivative thereof. In addition, it can express a cytokine, such as interleukin-2 (IL-2) or interleukin-12 (IL-12), which enhances the immune response (adjuvant effect). Thus, a vaccine vector can include an additional DNA molecule encoding, e.g., urease subunit A, B, or both, or a cytokine, placed under the control of elements required for expression in a mammalian cell.

Alternatively, a composition of the invention can include several vaccine vectors, each of them being capable of expressing a polypeptide or derivative of the invention. A composition can also contain a vaccine vector capable of expressing an additional Helicobacter antigen such as urease apoenzyme, a subunit, fragment, homolog, mutant, or derivative thereof; or a cytokine such as IL-2 or IL-12.

In vaccination methods for treating or preventing infection in a mammal, a vaccine vector of the invention can be administered by any conventional route in use in the vaccine field, particularly, to a mucosal (e.g., ocular, intranasal, oral, gastric, pulmonary, intestinal, rectal, vaginal, or urinary tract) surface or via the parenteral (e.g., subcutaneous, intradermal, intramuscular, intravenous, or intraperitoneal) route. Preferred routes depend upon the choice of the vaccine vector. The administration can be achieved in a single dose or repeated at intervals. The appropriate dosage depends on various parameters understood by skilled artisans such as the vaccine vector itself, the route of administration or the condition of the mammal to be vaccinated (weight, age and the like).

Live vaccine vectors available in the art include viral vectors such as adenoviruses and pox viruses as well as bacterial vectors, e.g., Shigella, Salmonella, Vibrio cholerae, Lactobacillus, Bacille bilié de Calmette-Guérin (BCG), and Streptococcus.

An example of an adenovirus vector, as well as a method for constructing an adenovirus vector capable of expressing a DNA molecule of the invention, are described in U.S. Pat. No. 4,920,209. Pox virus vectors that can be used include, e.g., vaccinia and canary pox virus, described in U.S. Pat. Nos. 4,722,848 and 5,364,773, respectively (also see, e.g., Tartaglia et al., Virology 188:217, 1992) for a description of a vaccinia virus vector; and Taylor et al, Vaccine 13:539, 1995, for a reference of a canary pox). Pox virus vectors capable of expressing a polynucleotide of the invention can be obtained by homologous recombination as described in Kieny et al., Nature 312:163, 1984, so that the polynucleotide of the invention is inserted in the viral genome under appropriate conditions for expression in mammalian cells. Generally, the dose of vaccine viral vector, for therapeutic or prophylactic use, can be of from about 1×10⁴ to about 1×10¹¹, advantageously from about 1×10⁷ to about 1×10¹⁰, preferably of from about 1×10⁷ to about 1×10⁹ plaque-forming units per kilogram. Preferably, viral vectors are administered parenterally; for example, in 3 doses, 4 weeks apart. Those skilled in the art recognize that it is preferable to avoid adding a chemical adjuvant to a composition containing a viral vector of the invention and thereby minimizing the immune response to the viral vector itself.

Non-toxicogenic Vibrio cholerae mutant strains that are useful as a live oral vaccine are described in Mekalanos et al., Nature 306:551, 1983, and U.S. Pat. No. 4,882,278 (strain in which a substantial amount of the coding sequence of each of the two ctxA alleles has been deleted so that no functional cholerae toxin is produced); WO 92/11354 (strain in which the irgA locus is inactivated by mutation; this mutation can be combined in a single strain with ctxA mutations); and WO 94/1533 (deletion mutant lacking functional ctxA and attRS1 DNA sequences). These strains can be genetically engineered to express heterologous antigens, as described in WO 94/19482. An effective vaccine dose of a Vibrio cholerae strain capable of expressing a polypeptide or polypeptide derivative encoded by a DNA molecule of the invention can contain, e.g., about 1×10⁵ to about 1×10⁹, preferably about 1×10⁶ to about 1×10⁸ viable bacteria in an appropriate volume for the selected route of administration. Preferred routes of administration include all mucosal routes; most preferably, these vectors are administered intranasally or orally.

Attenuated Salmonella typhimurium strains, genetically engineered for recombinant expression of heterologous antigens or not, and their use as oral vaccines are described in Nakayama et al. (Bio/Technology 6:693, 1988) and WO 92/11361. Preferred routes of administration include all mucosal routes; most preferably, these vectors are administered intranasally or orally.

Others bacterial strains useful as vaccine vectors are described in High et al., EMBO 11:1991, 1992, and Sizemore et al., Science 270:299, 1995 (Shigella flexneri); Medaglini et al., Proc. Natl. Acad. Sci. U.S.A. 92:6868, 1995 (Streptococcus gordonii); and Flynn, Cell. Mol. Biol. 40 (suppl. I):31, 1994, WO 88/6626, WO 90/0594, WO 91/13157, WO 92/1796, and WO 92/21376 (Bacille Calmette Guerin).

In bacterial vectors, polynucleotide of the invention can be inserted into the bacterial genome or can remain in a free state, carried on a plasmid.

An adjuvant can also be added to a composition containing a vaccine bacterial vector. A number of adjuvants are known to those skilled in the art. Preferred adjuvants can be selected from the list provided below.

According to a fourth aspect of the invention, there is also provided (i) a composition of matter containing a polynucleotide of the invention, together with a diluent or carrier; (ii) a pharmaceutical composition containing a therapeutically or prophylactically effective amount of a polynucleotide of the invention; (iii) a method for inducing an immune response against Helicobacter, in a mammal, by administering to the mammal, an immunogenically effective amount of a polynucleotide of the invention to elicit an immune response, e.g., a protective immune response to Helicobacter; and particularly, (iv) a method for preventing and/or treating a Helicobacter (e.g., H. pylori, H. felis, H. mustelae, or H. heilmanii) infection, by administering a prophylactic or therapeutic amount of a polynucleotide of the invention to an individual in need. Additionally, the fourth aspect of the invention encompasses the use of a polynucleotide of the invention in the preparation of a medicament for preventing and/or treating Helicobacter infection. The fourth aspect of the invention preferably includes the use of a DNA molecule placed under conditions for expression in a mammalian cell, e.g., in a plasmid that is unable to replicate in mammalian cells and to substantially integrate in a mammalian genome.

Polynucleotides (DNA or RNA) of the invention can also be administered as such to a mammal for vaccine, e.g., therapeutic or prophylactic, purpose. When a DNA molecule of the invention is used, it can be in the form of a plasmid that is unable to replicate in a mammalian cell and unable to integrate in the mammalian genome. Typically, a DNA molecule is placed under the control of a promoter suitable for expression in a mammalian cell. The promoter can function ubiquitously or tissue-specifically. Examples of non-tissue specific promoters include the early Cytomegalovirus (CMV) promoter (described in U.S. Pat. No. 4,168,062) and the Rous Sarcoma Virus promoter (described in Norton et al., Molec. Cell Biol. 5:281, 1985). The desmin promoter (Li et al., Gene 78:243, 1989, Li et al., J. Biol. Chem. 266:6562, 1991, and Li et al., J. Biol. Chem. 268:10403, 1993) is tissue-specific and drives expression in muscle cells. More generally, useful vectors are described, i.a., WO 94/21797 and Hartikka et al., Human Gene Therapy 7:1205, 1996.

For DNA/RNA vaccination, the polynucleotide of the invention can encode a precursor or a mature form. When it encodes a precursor form, the precursor form can be homologous or heterologous. In the latter case, a eucaryotic leader sequence can be used, such as the leader sequence of the tissue-type plasminogen factor (tPA).

A composition of the invention can contain one or several polynucleotides of the invention. It can also contain at least one additional polynucleotide encoding another Helicobacter antigen such as urease subunit A, B, or both; or a fragment, derivative, mutant, or analog thereof. A polynucleotide encoding a cytokine, such as interleukin-2 (IL-2) or interleukin-12 (IL-12), can also be added to the composition so that the immune response is enhanced. These additional polynucleotides are placed under appropriate control for expression. Advantageously, DNA molecules of the invention and/or additional DNA molecules to be included in the same composition, can be carried in the same plasmid.

Standard techniques of molecular biology for preparing and purifying polynucleotides can be used in the preparation of polynucleotide therapeutics of the invention. For use as a vaccine, a polynucleotide of the invention can be formulated according to various methods.

First, a polynucleotide can be used in a naked form, free of any delivery vehicles, such as anionic liposomes, cationic lipids, microparticles, e.g., gold microparticles, precipitating agents, e.g., calcium phosphate, or any other transfection-facilitating agent. In this case, the polynucleotide can be simply diluted in a physiologically acceptable solution, such as sterile saline or sterile buffered saline, with or without a carrier. When present, the carrier preferably is isotonic, hypotonic, or weakly hypertonic, and has a relatively low ionic strength, such as provided by a sucrose solution, e.g., a solution containing 20% sucrose.

Alternatively, a polynucleotide can be associated with agents that assist in cellular uptake. It can be, i.a., (i) complemented with a chemical agent that modifies the cellular permeability, such as bupivacaine (see, e.g., WO 94/16737), (ii) encapsulated into liposomes, or (iii) associated with cationic lipids or silica, gold, or tungsten microparticles.

Anionic and neutral liposomes are well known in the art (see, e.g., Liposomes: A Practical Approach, RPC New Ed, IRL press (1990), for a detailed description of methods for making liposomes) and are useful for delivering a large range of products, including polynucleotides.

Cationic lipids are also known in the art and are commonly used for gene delivery. Such lipids include Lipofectin™ also known as DOTMA (N-[1-(2,3-dioleyloxy)propyl]-N,N,N-trimethylammonium chloride), DOTAP (1,2-bis(oleyloxy)-3-(trimethylammonio)propane), DDAB (dimethyldioctadecylammonium bromide), DOGS (dioctadecylamidologlycyl spermine) and cholesterol derivatives such as DC-Chol (3 beta-(N-(N′,N′-dimethyl aminomethane)-carbamoyl) cholesterol). A description of these cationic lipids can be found in EP 187,702, WO 90/11092, U.S. Pat. No. 5,283,185, WO 91/15501, WO 95/26356, and U.S. Pat. No. 5,527,928. Cationic lipids for gene delivery are preferably used in association with a neutral lipid such as DOPE (dioleyl phosphatidylethanolamine), as, for example, described in WO 90/11092.

Other transfection-facilitating compounds can be added to a formulation containing cationic liposomes. A number of them are described in, e.g., WO 93/18759, WO 93/19768, WO 94/25608, and WO 95/2397. They include, i.a., spermine derivatives useful for facilitating the transport of DNA through the nuclear membrane (see, for example, WO 93/18759) and membrane-permeabilizing compounds such as GALA, Gramicidine S, and cationic bile salts (see, for example, WO 93/19768).

Gold or tungsten microparticles can also be used for gene delivery, as described in WO 91/359, WO 93/17706, and Tang et al. (Nature 356:152, 1992). In this case, the microparticle-coated polynucleotides can be injected via intradermal or intraepidermal routes using a needleless injection device (“gene gun”), such as those described in U.S. Pat. Nos. 4,945,050, 5,015,580, and WO 94/24263.

The amount of DNA to be used in a vaccine recipient depends, e.g., on the strength of the promoter used in the DNA construct, the immunogenicity of the expressed gene product, the condition of the mammal intended for administration (e.g., the weight, age, and general health of the mammal), the mode of administration, and the type of formulation. In general, a therapeutically or prophylactically effective dose from about 1 μg to about 1 mg, preferably, from about 10 μg to about 800 μg and, more preferably, from about 25 μg to about 250 μg, can be administered to human adults. The administration can be achieved in a single dose or repeated at intervals.

The route of administration can be any conventional route used in the vaccine field. As general guidance, a polynucleotide of the invention can be administered via a mucosal surface, e.g., an ocular, intranasal, pulmonary, oral, intestinal, rectal, vaginal, and urinary tract surface; or via a parenteral route, e.g., by an intravenous, subcutaneous, intraperitoneal, intradermal, intraepidermal, or intramuscular route. The choice of the administration route will depend on, e.g., the formulation that is selected. A polynucleotide formulated in association with bupivacaine is advantageously administered into muscles. When a neutral or anionic liposome or a cationic lipid, such as DOTMA or DC-Chol, is used, the formulation can be advantageously injected via intravenous, intranasal (aerosolization), intramuscular, intradermal, and subcutaneous routes. A polynucleotide in a naked form can advantageously be administered via the intramuscular, intradermal, or sub-cutaneous routes.

Although not absolutely required, such a composition can also contain an adjuvant. If so, a systemic adjuvant that does not require concomitant administration in order to exhibit an adjuvant effect is preferable such as, e.g., QS21, which is described in U.S. Pat. No. 5,057,546.

The sequence information provided in the present application enables the design of specific nucleotide probes and primers that can be useful in diagnosis. Accordingly, in a fifth aspect of the invention, there is provided a nucleotide probe or primer having a sequence found in or derived by degeneracy of the genetic code from a sequence shown in SEQ ID NO:1 to 47 (odd numbers).

The term “probe” as used in the present application refers to DNA (preferably single stranded) or RNA molecules (or modifications or combinations thereof) that hybridize under the stringent conditions, as defined above, to nucleic acid molecules having sequences homologous to those shown in SEQ ID NOs:1 to 47 (odd numbers), or to a complementary or anti-sense sequence. Generally, probes are significantly shorter than full-length sequences shown in SEQ ID NOs:1 to 47 (odd numbers); for example, they can contain from about 5 to about 100, preferably from about 10 to about 80 nucleotides. In particular, probes have sequences that are at least 75%, preferably at least 85%, more preferably 95% homologous to a portion of a sequence as shown in SEQ ID NOs:1 to 47 (odd numbers) or that are complementary to such sequences. Probes can contain modified bases such as inosine, methyl-5-deoxycytidine, deoxyuridine, dimethylamino-5-deoxyuridine, or diamino-2,6-purine. Sugar or phosphate residues can also be modified or substituted. For example, a deoxyribose residue can be replaced by a polyamide (Nielsen et al., Science 254:1497, 1991) and phosphate residues can be replaced by ester groups such as diphosphate, alkyl, arylphosphonate and phosphorothioate esters. In addition, the 2′-hydroxyl group on ribonucleotides can be modified by including, e.g., alkyl groups.

Probes of the invention can be used in diagnostic tests, as capture or detection probes. Such capture probes can be conventionally immobilized on a solid support, directly or indirectly, by covalent means or by passive adsorption. A detection probe can be labeled by a detection marker selected from radioactive isotopes; enzymes such as peroxidase, alkaline phosphatase, and enzymes able to hydrolyze a chromogenic, fluorogenic, or luminescent substrate; compounds that are chromogenic, fluorogenic, or luminescent; nucleotide base analogs; and biotin.

Probes of the invention can be used in any conventional hybridization technique, such as dot blot (Maniatis et al., Molecular Cloning: A Laboratory Manual (1982) Cold Spring Harbor Laboratory Press, Cold Spring Harbor, N.Y.), Southern blot (Southern, J. Mol. Biol. 98:503, 1975), northern blot (identical to Southern blot to the exception that RNA is used as a target), or the sandwich technique (Dunn et al., Cell 12:23, 1977). The latter technique involves the use of a specific capture probe and/or a specific detection probe with nucleotide sequences that at least partially differ from each other.

A primer is usually a probe of about 10 to about 40 nucleotides that is used to initiate enzymatic polymerization of DNA in an amplification process (e.g., PCR), in an elongation process, or in a reverse transcription method. In a diagnostic method involving PCR, primers can be labeled.

Thus, the invention also encompasses (i) a reagent containing a probe of the invention for detecting and/or identifying the presence of Helicobacter in a biological material; (ii) a method for detecting and/or identifying the presence of Helicobacter in a biological material, in which (a) a sample is recovered or derived from the biological material, (b) DNA or RNA is extracted from the material and denatured, and (c) exposed to a probe of the invention, for example, a capture, detection probe or both, under stringent hybridization conditions, such that hybridization is detected; and (iii) a method for detecting and/or identifying the presence of Helicobacter in a biological material, in which (a) a sample is recovered or derived from the biological material, (b) DNA is extracted therefrom, (c) the extracted DNA is primed with at least one, and preferably two, primers of the invention and amplified by polymerase chain reaction, and (d) the amplified DNA fragment is produced.

As previously mentioned, polypeptides that can be produced upon expression of the newly identified open reading frames are useful vaccine agents.

Therefore, a sixth aspect of the invention features a substantially purified polypeptide or polypeptide derivative having an amino acid sequence encoded by a polynucleotide of the invention.

A “substantially purified polypeptide” is defined as a polypeptide that is separated from the environment in which it naturally occurs and/or that is free of the majority of the polypeptides that are present in the environment in which it was synthesized. For example, a substantially purified polypeptide is free from cytoplasmic polypeptides. A substantiall purified polypeptide can be, for example, at least 60%, 70%, 80%, 90%, 95%, or 100% pure, with respect to, for example, other Helicobacter components. Those skilled in the art will understand that the polypeptides of the invention can be purified from a natural source, i.e., a Helicobacter strain, or can be produced by recombinant means.

Homologous polypeptides or polypeptide derivatives encoded by polynucleotides of the invention can be screened for specific antigenicity by testing cross-reactivity with an antiserum raised against the polypeptide of reference having an amino acid sequence as shown in SEQ ID NOs:2 to 48 (even numbers) or encoded by one of the deposited DNA molecules. Briefly, a monospecific hyperimmune antiserum can be raised against a purified reference polypeptide as such or as a fusion polypeptide, for example, an expression product of MBP, GST, or His-tag systems or a synthetic peptide predicted to be antigenic. The homologous polypeptide or derivative screened for specific antigenicity can be produced as such or as a fusion polypeptide. In this latter case and if the antiserum is also raised against a fusion polypeptide, two different fusion systems are employed. Specific antigenicity can be determined according to a number of methods, including Western blot (Towbin et al., Proc. Natl. Acad. Sci. U.S.A. 76:4350, 1979), dot blot, and ELISA, as described below.

In a Western blot assay, the product to be screened, either as a purified preparation or a total E. coli extract, is submitted to SDS-Page electrophoresis as described by Laemmli (Nature 227:680, 1970). After transfer to a nitrocellulose membrane, the material is further incubated with the monospecific hyperimmune antiserum diluted in the range of dilutions from about 1:50 to about 1:5000, preferably from about 1:100 to about 1:500. Specific antigenicity is shown once a band corresponding to the product exhibits reactivity at any of the dilutions in the above range.

In an ELISA assay, the product to be screened is preferably used as the coating antigen. A purified preparation is preferred, although a whole cell extract can also be used. Briefly, about 100 μl of a preparation at about 10 μg protein/ml are distributed into wells of a 96-well polycarbonate ELISA plate. The plate is incubated for 2 hours at 37° C. then overnight at 4° C. The plate is washed with phosphate buffer saline (PBS) containing 0.05% Tween 20 (PBS/Tween buffer). The wells are saturated with 250 μl PBS containing 1% bovine serum albumin (BSA) to prevent non-specific antibody binding. After 1 hour of incubation at 37° C., the plate is washed with PBS/Tween buffer. The antiserum is serially diluted in PBS/Tween buffer containing 0.5% BSA. 100 μl of dilutions are added per well. The plate is incubated for 90 minutes at 37° C., washed and evaluated according to standard procedures. For example, a goat anti-rabbit peroxidase conjugate is added to the wells when specific antibodies were raised in rabbits. Incubation is carried out for 90 minutes at 37° C. and the plate is washed. The reaction is developed with the appropriate substrate and the reaction is measured by colorimetry (absorbance measured spectrophotometrically). Under the above experimental conditions, a positive reaction is shown once an O.D. value of 1.0 is associated with a dilution of at least about 1:50, preferably of at least about 1:500.

In a dot blot assay, a purified product is preferred, although a whole cell extract can also be used. Briefly, a solution of the product at about 100 μg/ml is serially two-fold diluted in 50 mM Tris-HCl (pH 7.5). 100 μl of each dilution are applied to a nitrocellulose membrane 0.45 μm set in a 96-well dot blot apparatus (Biorad). The buffer is removed by applying vacuum to the system. Wells are washed by addition of 50 mM Tris-HCl (pH 7.5) and the membrane is air-dried. The membrane is saturated in blocking buffer (50 mM Tris-HCl (pH 7.5) 0.15 M NaCl, 10 μg/L skim milk) and incubated with an antiserum dilution from about 1:50 to about 1:5000, preferably about 1:500. The reaction is revealed according to standard procedures. For example, a goat anti-rabbit peroxidase conjugate is added to the wells when rabbit antibodies are used. Incubation is carried out 90 minutes at 37° C. and the blot is washed. The reaction is developed with the appropriate substrate and stopped. The reaction is measured visually by the appearance of a colored spot, e.g., by colorimetry. Under the above experimental conditions, a positive reaction is shown once a colored spot is associated with a dilution of at least about 1:50, preferably of at least about 1:500.

Therapeutic or prophylactic efficacy of a polypeptide or derivative of the invention can be evaluated as described below.

According to a seventh aspect of the invention, there is provided (i) a composition of matter containing a polypeptide of the invention together with a diluent or carrier; in particular, (ii) a pharmaceutical composition containing a therapeutically or prophylactically effective amount of a polypeptide of the invention; (iii) a method for inducing an immune response against Helicobacter in a mammal, by administering to the mammal an immunogenically effective amount of a polypeptide of the invention to elicit an immune response, e.g., a protective immune response to Helicobacter; and particularly, (iv) a method for preventing and/or treating a Helicobacter (e.g., H. pylori, H. felis, H. mustelae, or H. heilmanii) infection, by administering a prophylactic or therapeutic amount of a polypeptide of the invention to an individual in need. Additionally, the seventh aspect of the invention encompasses the use of a polypeptide of the invention in the preparation of a medicament for preventing and/or treating Helicobacter infection.

The immunogenic compositions of the invention can be administered by any conventional route in use in the vaccine field, in particular to a mucosal (e.g., ocular, intranasal, pulmonary, oral, gastric, intestinal, rectal, vaginal, or urinary tract) surface or via the parenteral (e.g., subcutaneous, intradermal, intramuscular, intravenous, or intraperitoneal) route. The choice of the administration route depends upon a number of parameters, such as the adjuvant associated with the polypeptide. For example, if a mucosal adjuvant is used, the intranasal or oral route will be preferred and if a lipid formulation or an aluminum compound is used, the parenteral route will be preferred. In the latter case, the subcutaneous or intramuscular route is most preferred. The choice can also depend upon the nature of the vaccine agent. For example, a polypeptide of the invention fused to CTB or LTB will be best administered to a mucosal surface.

A composition of the invention can contain one or several polypeptides or derivatives of the invention. It can also contain at least one additional Helicobacter antigen such as the urease apoenzyme or a subunit, fragment, homolog, mutant, or derivative thereof.

For use in a composition of the invention, a polypeptide or derivative thereof can be formulated into or with liposomes, preferably neutral or anionic liposomes, microspheres, ISCOMS, or virus-like-particles (VLPs) to facilitate delivery and/or enhance the immune response. These compounds are readily available to one skilled in the art; for example, see Liposomes: A Practical Approach (supra).

Adjuvants other than liposomes and the like can also be used and are known in the art. An appropriate selection can conventionally be made by those skilled in the art, for example, from the list provided below.

Administration can be achieved in a single dose or repeated as necessary at intervals as can be determined by one skilled in the art. For example, a priming dose can be followed by three booster doses at weekly or monthly intervals. An appropriate dose depends on various parameters including the recipient (e.g., adult or infant), the particular vaccine antigen, the route and frequency of administration, the presence/absence or type of adjuvant, and the desired effect (e.g., protection and/or treatment), as can be determined by one skilled in the art. In general, a vaccine antigen of the invention can be administered by a mucosal route in an amount from about 10 μg to about 500 mg, preferably from about 1 mg to about 200 mg. For the parenteral route of administration, the dose usually should not exceed about 1 mg, preferably about 100 μg.

When used as vaccine agents, polynucleotides and polypeptides of the invention can be used sequentially as part of a multistep immunization process. For example, a mammal can be initially primed with a vaccine vector of the invention such as a pox virus, e.g., via the parenteral route, and then boosted twice with the polypeptide encoded by the vaccine vector, e.g., via the mucosal route. In another example, liposomes associated with a polypeptide or derivative of the invention can also be used for priming, with boosting being carried out mucosally using a soluble polypeptide or derivative of the invention in combination with a mucosal adjuvant (e.g., LT).

A polypeptide derivative of the invention is also useful as a diagnostic reagent for detecting the presence of anti-Helicobacter antibodies, e.g., in a blood sample. Such polypeptides are about 5 to about 80, preferably about 10 to about 50 amino acids in length and can be labeled or unlabeled, depending upon the diagnostic method. Diagnostic methods involving such a reagent are described below.

Upon expression of a DNA molecule of the invention, a polypeptide or polypeptide derivative is produced and can be purified using known laboratory techniques. For example, the polypeptide or polypeptide derivative can be produced as a fusion protein containing a fused tail that facilitates purification. The fusion product can be used to immunize a small mammal, e.g., a mouse or a rabbit, in order to raise antibodies against the polypeptide or polypeptide derivative (monospecific antibodies). The eighth aspect of the invention thus provides a monospecific antibody that binds to a polypeptide or polypeptide derivative of the invention.

By “monospecific antibody” is meant an antibody that is capable of reacting with a unique naturally-occuring Helicobacter polypeptide. An antibody of the invention can be polyclonal or monoclonal. Monospecific antibodies can be recombinant, e.g., chimeric (e.g., constituted by a variable region of murine origin associated with a human constant region), humanized (a human immunoglobulin constant backbone together with hypervariable region of animal, e.g., murine, origin), and/or single chain. Both polyclonal and monospecific antibodies can also be in the form of immunoglobulin fragments, e.g., F(ab)′2 or Fab fragments. The antibodies of the invention can be of any isotype, e.g., IgG or IgA, and polyclonal antibodies can be of a single isotype or can contain a mixture of isotypes.

The antibodies of the invention, which are raised to a polypeptide or polypeptide derivative of the invention, can be produced and identified using standard immunological assays, e.g., Western blot analysis, dot blot assay, or ELISA (see, e.g., Coligan et al., Current Protocols in Immunology (1994) John Wiley & Sons, Inc., New York, N.Y.). The antibodies can be used in diagnostic methods to detect the presence of a Helicobacter antigen in a sample, such as a biological sample. The antibodies can also be used in affinity chromatography methods for purifying a polypeptide or polypeptide derivative of the invention. As is discussed further below, such antibodies can be used in prophylactic and therapeutic passive immunization methods.

Accordingly, a ninth aspect of the invention provides (i) a reagent for detecting the presence of Helicobacter in a biological sample that contains an antibody, polypeptide, or polypeptide derivative of the invention; and (ii) a diagnostic method for detecting the presence of Helicobacter in a biological sample, by contacting the biological sample with an antibody, a polypeptide, or a polypeptide derivative of the invention, such that an immune complex is formed, and by detecting such complex to indicate the presence of Helicobacter in the sample or the organism from which the sample is derived.

Those skilled in the art will understand that the immune complex is formed between a component of the sample and the antibody, polypeptide, or polypeptide derivative, whichever is used, and that any unbound material can be removed prior to detecting the complex. As can be easily understood, a polypeptide reagent is useful for detecting the presence of anti-Helicobacter antibodies in a sample, e.g., a blood sample, while an antibody of the invention can be used for screening a sample, such as a gastric extract or biopsy, for the presence of Helicobacter polypeptides.

For use in diagnostic applications, the reagent (i.e., the antibody, polypeptide, or polypeptide derivative of the invention) can be in a free state or immobilized on a solid support, such as a tube, a bead, or any other conventional support used in the field. Immobilization can be achieved using direct or indirect means. Direct means include passive adsorption (non-covalent binding) or covalent binding between the support and the reagent. By “indirect means” is meant that an anti-reagent compound that interacts with a reagent is first attached to the solid support. For example, if a polypeptide reagent is used, an antibody that binds to it can serve as an anti-reagent, provided that it binds to an epitope that is not involved in the recognition of antibodies in biological samples. Indirect means can also employ a ligand-receptor system, for example, a molecule such as a vitamin can be grafted onto the polypeptide reagent and the corresponding receptor can be immobilized on the solid phase. This is illustrated by the biotin-streptavidin system. Alternatively, indirect means can be used, e.g., by adding to the reagent a peptide tail, chemically or by genetic engineering, and immobilizing the grafted or fused product by passive adsorption or covalent linkage of the peptide tail.

According to a tenth aspect of the invention, there is provided a process for purifying, from a biological sample, a polypeptide or polypeptide derivative of the invention, which involves carrying out antibody-based affinity chromatography with the biological sample, wherein the antibody is a monospecific antibody of the invention.

For use in a purification process of the invention, the antibody can be polyclonal or monospecific, and preferably is of the IgG type. Purified IgGs can be prepared from an antiserum using standard methods (see, e.g., Coligan et al., supra). Conventional chromatography supports, as well as standard methods for grafting antibodies, are disclosed in, e.g., Antibodies: A Laboratory Manual, D. Lane, E. Harlow, Eds. (1988).

Briefly, a biological sample, such as an H. pylori extract, preferably in a buffer solution, is applied to a chromatography material, preferably equilibrated with the buffer used to dilute the biological sample so that the polypeptide or polypeptide derivative of the invention (i.e., the antigen) is allowed to adsorb onto the material. The chromatography material, such as a gel or a resin coupled to an antibody of the invention, can be in batch form or in a column. The unbound components are washed off and the antigen is then eluted with an appropriate elution buffer, such as a glycine buffer or a buffer containing a chaotropic agent, e.g., guanidine HCl, or high salt concentration (e.g., 3 M MgCl₂). Eluted fractions are recovered and the presence of the antigen is detected, e.g., by measuring the absorbance at 280 nm.

An antibody of the invention can be screened for therapeutic efficacy as described as follows. According to an eleventh aspect of the invention, there is provided (i) a composition of matter containing a monospecific antibody of the invention, together with a diluent or carrier; (ii) a pharmaceutical composition containing a therapeutically or prophylactically effective amount of a monospecific antibody of the invention, and (iii) a method for treating or preventing a Helicobacter (e.g., H. pylori, H. felis, H. mustelae, or H. heilmanii) infection, by administering a therapeutic or prophylactic amount of a monospecific antibody of the invention to an individual in need. Additionally, the eleventh aspect of the invention encompasses the use of a monospecific antibody of the invention in the preparation of a medicament for treating or preventing Helicobacter infection.

To this end, the monospecific antibody can be polyclonal or monoclonal, preferably of the IgA isotype (predominantly). In passive immunization, the antibody can be administered to a mucosal surface of a mammal, e.g., the gastric mucosa, e.g., orally or intragastrically, advantageously, in the presence of a bicarbonate buffer. Alternatively, systemic administration, not requiring a bicarbonate buffer, can be carried out. A monospecific antibody of the invention can be administered as a single active component or as a mixture with at least one monospecific antibody specific for a different Helicobacter polypeptide. The amount of antibody and the particular regimen used can readily be determined by those skilled in the art. For example, daily administration of about 100 to 1,000 mg of antibodies over one week, or three doses per day of about 100 to 1,000 mg of antibodies over two or three days, can be an effective regimens for most purposes.

Therapeutic or prophylactic efficacy can be evaluated using standard methods in the art, e.g., by measuring induction of a mucosal immune response or induction of protective and/or therapeutic immunity, using, e.g., the H. felis mouse model and the procedures described in Lee et al. (Eur. J. Gastroenterology and Hepatology 7:303, 1995) or Lee et al. (J. Infect. Dis. 172:161, 1995). Those skilled in the art will recognize that the H. felis strain of the model can be replaced with another Helicobacter strain. For example, the efficacy of DNA molecules and polypeptides from H. pylori is preferably evaluated in a mouse model using an H. pylori strain. Protection can be determined by comparing the degree of Helicobacter infection in the gastric tissue (assessed by urease activity, bacterial counts or gastritis) to that of a control group. Protection is shown when infection is reduced by comparison to the control group. Such an evaluation can be made for polynucleotides, vaccine vectors, polypeptides and derivatives thereof, as well as antibodies of the invention.

For example, various doses of an antibody of the invention can be administered to the gastric mucosa of mice previously challenged with an H. pylori strain, as described, e.g., in Lee et al (supra). Then, after an appropriate period of time, the bacterial load of the mucosa is estimated by assessing the urease activity, as compared to a control. Reduced urease activity indicates that the antibody is therapeutically effective.

Adjuvants useful in any of the vaccine compositions described above are as follows.

Adjuvants for parenteral administration include aluminum compounds, such as aluminum hydroxide, aluminum phosphate, and aluminum hydroxy phosphate. The antigen can be precipitated with, or adsorbed onto, the aluminum compound according to standard protocols. Other adjuvants, such as RIBI (ImmunoChem, Hamilton, Mont.), can be used in parenteral administration.

Adjuvants for mucosal administration include bacterial toxins, e.g., the cholera toxin (CT), the E. coli heat-labile toxin (LT), the Clostridium difficile toxin A and the pertussis toxin (PT), or combinations, subunits, toxoids, or mutants thereof. For example, a purified preparation of native cholera toxin subunit B (CTB) can be of use. Fragments, homologs, derivatives, and fusions to any of these toxins are also suitable, provided that they retain adjuvant activity. Preferably, a mutant having reduced toxicity is used. Suitable mutants are described, e.g., in WO 95/17211 (Arg-7-Lys CT mutant), WO 96/6627 (Arg-192-Gly LT mutant), and WO 95/34323 (Arg-9-Lys and Glu-129-Gly PT mutant). Additional LT mutants that can be used in the methods and compositions of the invention include, e.g., Ser-63-Lys, Ala-69-Gly, Glu-110-Asp, and Glu-112-Asp mutants. Other adjuvants, such as a bacterial monophosphoryl lipid A (MPLA) of, e.g., E. coli, Salmonella minnesota, Salmonella typhimurium, or Shigella flexneri; saponins, or polylactide glycolide (PLGA) microspheres, can also be used in mucosal administration.

Adjuvants useful for both mucosal and parenteral administrations include polyphosphazene (WO 95/2415), DC-chol (3 β-(N-(N′,N′-dimethyl aminomethane)-carbamoyl) cholesterol; U.S. Pat. No. 5,283,185 and WO 96/14831) and QS-21 (WO 88/9336).

Any pharmaceutical composition of the invention, containing a polynucleotide, a polypeptide, a polypeptide derivative, or an antibody of the invention, can be manufactured in a conventional manner. In particular, it can be formulated with a pharmaceutically acceptable diluent or carrier, e.g., water or a saline solution such as phosphate buffer saline, optionally complemented with a bicarbonate salt, such as sodium bicarbonate, e.g., 0.1 to 0.5 M. Bicarbonate can be advantageously added to compositions intended for oral or intragastric administration. In general, a diluent or carrier can be selected on the basis of the mode and route of administration, and standard pharmaceutical practice. Suitable pharmaceutical carriers or diluents, as well as pharmaceutical necessities for their use in pharmaceutical formulations, are described in Remington's Pharmaceutical Sciences, a standard reference text in this field and in the USP/NF.

The invention also includes methods in which gastroduodenal infections, such as Helicobacter infection, are treated by oral administration of a Helicobacter polypeptide of the invention and a mucosal adjuvant, in combination with an antibiotic, an antisecretory agent, a bismuth salt, an antacid, sucralfate, or a combination thereof. Examples of such compounds that can be administered with the vaccine antigen and the adjuvant are antibiotics, including, e.g., macrolides, tetracyclines, β-lactams, aminoglycosides, quinolones, penicillins, and derivatives thereof (specific examples of antibiotics that can be used in the invention include, e.g., amoxicillin, clarithromycin, tetracycline, metronidizole, erythromycin, cefuroxime, and erythromycin); antisecretory agents, including, e.g., H₂-receptor antagonists (e.g., cimetidine, ranitidine, famotidine, nizatidine, and roxatidine), proton pump inhibitors (e.g., omeprazole, lansoprazole, and pantoprazole), prostaglandin analogs (e.g., misoprostil and enprostil), and anticholinergic agents (e.g., pirenzepine, telenzepine, carbenoxolone, and proglumide); and bismuth salts, including colloidal bismuth subcitrate, tripotassium dicitrate bismuthate, bismuth subsalicylate, bicitropeptide, and pepto-bismol (see, e.g., Goodwin et al., Helicobacter pylori, Biology and Clinical Practice, CRC Press, Boca Raton, Fla., pp 366-395, 1993; Physicians' Desk Reference, 49^{th} edn., Medical Economics Data Production Company, Montvale, N.J., 1995). In addition, compounds containing more than one of the above-listed components coupled together, e.g., ranitidine coupled to bismuth subcitrate, can be used. The invention also includes compositions for carrying out these methods, i.e., compositions containing a Helicobacter antigen (or antigens) of the invention, an adjuvant, and one or more of the above-listed compounds, in a pharmaceutically acceptable carrier or diluent.

Amounts of the above-listed compounds used in the methods and compositions of the invention can readily be determined by those skilled in the art. In addition, one skilled in the art can readily design treatment/immunization schedules. For example, the non-vaccine components can be administered on days 1-14, and the vaccine antigen+adjuvant can be administered on days 7, 14, 21, and 28.

Methods and pharmaceutical compositions of the invention can be used to treat or prevent Helicobacter infections and, accordingly, gastroduodenal diseases associated with these infections, including acute, chronic, and atrophic gastritis; and peptic ulcer diseases, e.g., gastric and duodenal ulcers.

All twenty-four clones of the invention were isolated by a transposon shuttle mutagenesis method. Briefly, in this method, a TnMax9 mini-blaM transposon was used for insertional mutagenesis of an H. pylori gene library established in E. coli. 192 E. coli clones expressing active β-lactamase fusion proteins were obtained, indicating that the corresponding target plasmids carry H. pylori genes encoding extracytoplasmic proteins. Individual mutants were transferred onto the chromosome of H. pylori P1 or P12 by natural transformation, resulting in 135 distinct H. pylori mutants. This method is described in further detail, as follows.

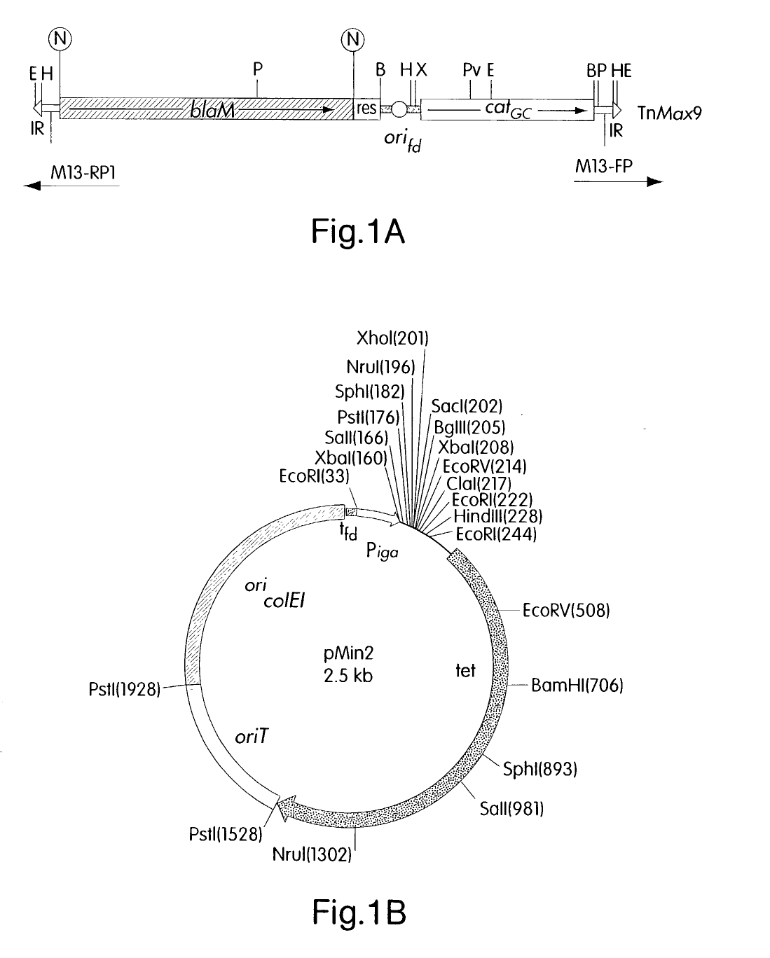

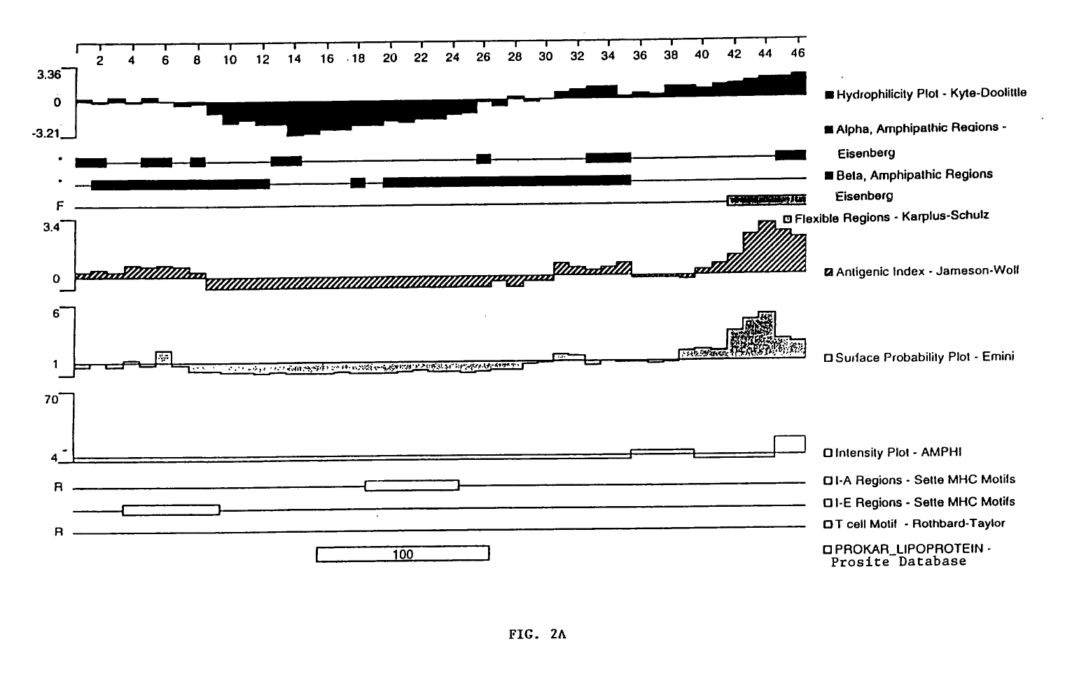

The transposon TnMax9 (Kahrs et al., Gene 167:53, 1995) was used to generate mutations in an H. pylori library in E. coli. As illustrated in FIG. 1A, TnMax9 contains, in addition to a cat_{GC}-resistance gene close to the inverted repeat (IR), an unexpressed open reading frame encoding β-lactamase without a promoter or leader sequence (mature β-lactamase, blaM; Kahrs et al., supra). For production of extracytoplasmic BlaM fusion proteins resulting in ampicillin-resistant (amp^{R}) clones, expression of the cloned H. pylori genes in E. coli is obligatory. The minimal vector pMin2 (Kahrs et al., supra; see FIG. 1B), containing a weak constitutive promoter (P_{iga}) upstream of the multiple cloning site, was used for construction of the H. pylori library to ensure expression of H. pylori genes in E. coli.

In construction of the library, H. pylori DNA was partially digested with Sau3A and HpaII, size fractionated by preparative agarose gel electrophoresis, and 3-6 kb fragments were ligated into the BglII and ClaI sites of pMin2. The library was introduced into E. coli strain E181(pTnMax9), which is a derivative of HB101 containing the TnMax9 transposon, by electroporation. This generated approximately 2,400 independent transformants. More than 95% of the plasmids contained an insert of between 3 and 6 kb, showing that the 1.7 Mb H. pylori chromosome was statistically covered. Since not every plasmid could be expected to contain a target gene carrying an export signal, the library was partitioned into a total of 198 pools (24 pools of 20 clones and 174 pools of 11 clones). Using a cotton swab, either eleven or twenty individual colonies were inoculated in 0.5 ml LB medium in a eppendorf tubes, vortexed, and 100 ml of the suspension was spread on LB agar plates supplemented with tetracycline and chloramphenicol to select for maintenance of both plasmids. Insertion of TnMax9 into the target plasmids was induced with 100 mM isopropyl-b-D-thiogalactoside (IPTG) separately for each pool (Haas et al., Gene 130:23-21, 1993). Plasmids were transferred into E145 by triparental mating, in which 25 ml of the donor strain (E181), 25 ml of the mobilisator (KB101(pRK2013)), and 50 ml of the recipient strain (E145) were mixed from corresponding bacterial suspensions (O.D.₅₅₀=10). The matings were performed for 2-3 hours at 37° C. on nitrocellulose filters, which were placed on LB plates. Bacteria were suspended in 1 ml LB and aliquots were spread on LB plates containing chloramphenicol, tetracycline, and rifampicin. Each pool gave rise to chloramphenicol-resistant transconjugates in E145, demonstrating that both transposition and conjugation were successful. Generally, several thousand chloramphenicol-resistant transconjugates were obtained, but the number of amp^{R} colonies varied in different pools, ranging from one to several hundred colonies. Two amp^{R} colonies from each positive pool were isolated, plasmid DNA was extracted, and the DNA was characterized by further restriction analysis. Only those TnMax9 insertions of a single pool that mapped in obviously different plasmid clones, or in markedly different regions of the same clone, were used further.

From 158 of the 198 pools, ampicillin-resistant E145 transconjugates were obtained (80%), showing that in several pools, TnMax9 inserted into expressed genes, resulting in production of extracytoplasmic BlaM fusion proteins. Thus, a total of 192 amp^{R} E145 clones could be isolated by conjugal transfer of plasmids from 198 pools.

To analyze the mutant library, it was determined whether defined gene sequences inactivated by TnMax9 were represented once or several times in the whole library. Five transposon-containing plasmids conferring an amp^{R} phenotype to E145 (pMu7, pMu13, pMu75, pMu94, and pMu110) were randomly selected and DNA fragments flanking the TnMax9 insert were isolated and used as probes in Southern hybridization of 120 amp^{R} clones. The hybridization probes isolated from clones pMu7, pMu75, and pMu94 were between 0.9 and 1.1 kb in size, and hybridized exclusively with the inserts of the homologous plasmids. In contrast, the TnMax9 flanking regions of clones pMu13 and pMu110 were 4.0 kb and 5.5 kb, respectively. They each hybridized with the homologous plasmids, and with one additional clone of the library. Such a result was expected, since the chance of a probe to find a homologous sequence in the library should be higher, the longer the hybridization probes.

In order to verify the insertion of the transposon into distinct ORFs encoding putative exported proteins, the TnMax9-flanking DNA of five representative amp^{R} mutant clones (pMu7, pMu12, pMu18, pMu20, and pMu26) was sequenced, taking advantage of the M13 forward and reverse primers on TnMax9 (FIG. 1A). This analysis revealed that the mini-transposon was inserted into different sequences in each plasmid, thereby interrupting ORFs encoding putative proteins. For two clones, the sequences located upstream of the blaM gene revealed a putative ribosome-binding site and a potential translational start codon (ATG). Other clones either revealed an ORF spanning the complete sequence (approximately 400 basepairs upstream and downstream of the TnMax9 insertion) or termninating shortly after the site of TnMax9 insertion. The partial protein sequences from different ORFs were used for database searches, but no significant homologies with known proteins were found.

In a further approach, it was determined whether a known gene, like vacA, encoding the extracellular vacuolating cytotoxin of H. pylori, could be identified using this method and how often such a mutation would be represented in the mutant library. A total cell lysates of the 135 mutants were tested in an immunoblot using the H. pylori cytotoxin-specific rabbit antiserum AK197 (Schmitt et al., Mol. Microbiol. 12:307-319, 1994). Two mutants were identified, which no longer produced the cytotoxin antigen (mutants P1-26 and P1-47) and partial DNA sequencing of the insertion sites revealed that TnMax9 was inserted at distinct positions in the vacA gene, 56 and 53 codons downstream of the ATG start codon, respectively.

Thus, the characterization of the mutant collection confirmed that a representative gene library was constructed in E. coli, in which target genes encoding exported H. pylori proteins were efficiently tagged by TnMax9.

In order to establish a collection of mutants lacking distinct exported proteins, the mutations had to be transferred back into the H. pylori chromosome. By means of natural transformation, 86 plasmids could be transformed into the original strain P1. H. pylori strains P1 or P12, which were naturally competent for DNA transformation, were transformed with circular plasmid DNA (0.2-0.5 mg/transformation). Transformations to streptomycin resistance were performed with chromosomal DNA (1 mg/transformation), isolated from a streptomycin-resistant NCTC11637H. pylori mutant according to the procedure described in Haas et al. (Mol. Microbiol. 8:753-760). Selection was performed on serum plates containing 4 mg/ml chloramphenicol or 500 mg/ml streptomycin. The transformation frequency for a given mutant was calculated as the number of chloramphenicol-, streptomycin-, or erythromycin-resistant colonies per cfu (average of three experiments). The blaM gene was deleted by NotI digestion, and the plasmid religated, in those plasmids that did not transform strain P1 directly. This procedure, which resulted in a twenty to thirty-fold higher frequency of transformation, as compared to the same plasmid containing blaM, resulted in 36 additional mutants strain P1. The blaM-deletion plasmids that still did not transform strain P1 were used to transform the heterologous H. pylori strain P12, possessing an approximately 10-fold higher transformation frequency compared to P1. This resulted in thirteen further mutants.

Thus, from the 192 amp^{R} plasmids a total of 135H. pylori mutants (122 mutants in P1 and 13 mutants in P12) were finally obtained by selection on chloramphenicol resistance (70%). The transformation frequency varied between different plasmids in the range of 1×10⁻⁵-1×10⁻⁷. The remaining plasmids did not result in any transformants. The collection was frozen as individual mutants in stock cultures at −70° C. To verify the correct insertion of the mini-transposon into the H. pylori chromosome, ten representative mutants were tested by Southern hybridization of chromosomal DNA using cat_{GC} DNA and the vector pMin2 as probes. Consistent with our previous experience concerning TnMax9-based shuttle mutagenesis of H. pylori, the mini-transposon was, in all cases, inserted into the chromosome without integration of the vector DNA, which probably means by a double cross-over, rather than by a single cross-over event. As judged from the hybridization pattern obtained with the cat gene as a probe, it appears that TnMax9 is located in different regions of the chromosome, showing that distinct target genes have been interrupted in individual mutants.

The mutants were analyzed for motility, transformation competence, and adherence to KatoIII cells. Screening of the H. pylori mutant collection allowed identification of mutants impaired in motility, natural transformation competence, and adherence to gastric epithelial cell lines. Motility mutants could be grouped into distinct classes: (i) mutants lacking the major flagellin subunit FlaA and intact flagella; (ii) mutants with apparently normal flagella, but reduced motility; and (iii) mutants with obviously normal flagella, but completely abolished motility. Two independent mutations, which exhibited defects in natural competence for genetic transformation, mapped to different genetic loci. In addition, two independent mutants were isolated by their failure to bind to the human gastric carcinoma cell line KatoIII. Both mutants carried a transposon in the same gene, approximately 0.8 kb apart, and showed decrease autoagglutination, when compared to the wild type strain.

The invention is further illustrated by the following examples. Example 1 describes isolation of DNA encoding a polypeptide of the invention, HPO76. The methods described in Example 1 can be adapted for isolating nucleic acids encoding the other polypeptides of the invention. Example 2 describes methods for obtaining the nucleic acids of the invention from the deposited clones.

### EXAMPLE 1

### Preparation of Isolated DNA Encoding HPO76

1.A. Preparation of Genomic DNA from Helicobacter Pylori

Helicobacter pylori strain ORV2001, stored in LB medium containing 50% glycerol at −70° C., is grown on Colombia agar containing 7% sheep blood for 48 hours under microaerophilic conditions (8-10% CO₂, 5-7% O₂, 85-87% N₂). Cells are harvested, washed with phosphate buffer saline (PBS; pH 7.2), and DNA is then extracted using the Rapid Prep Genomic DNA Isolation kit (Pharmacia Biotech).

1.B. PCR Amplification

The DNA fragment is amplified from genomic DNA, as prepared above, by the Polymerase Chain Reaction (PCR) using the following primers:

| | | |
|---|---|---|
| -N-terminal primer: | 5′-GCC[GAGCTC]ITATCGTATGGACTTAGAACAT-3′ | (SEQ ID NO:145) |
| -C-terminal primer: | 5′-GCC[CTCGAG]ATTAGAATAAGTGTTGTTTAAAATC-3′. | (SEQ ID NO:146) |

Both primers include a clamp (GCC) and a restriction enzyme recognition sequence for cloning purposes (SacI (GAGCTC) and XhoI (CTCGAG) recognition sequences). The underlined sequences in both primers represent clone 76-specific sequences. The N-terminal primer is designed so that the amplified product does not encode the leader sequence and the potential cleavage site.

Amplification of gene-specific DNA is carried out using Pwo DNA Polymerase (Boehringer Mannheim), which is a proof-reading polymerase, according to general guidance provided by the manufacturer. Because of the exonuclease activity of the polymerase, two reaction mixtures (mixtures 1 and 2) are first prepared separately and combined just prior to amplification. These mixtures are as follows:

| Ingredient (final conc.) | Mixture 1 (l) | Mixture 2 (l) |
|---|---|---|
| distilled H₂O | 160 | 79 |
| dNTPs (200 M each) | 40 | — |
| 10x PCR buffer | — | 20 |
| primers (100 nM each) | 1 | — |
| DNA template (200 ng) | 2 | — |
| as obtained in 1.A. | | |

(10x PCR buffer contains 100 mM Tris-HCl (pH 8.85), 250 mM KCl, 50 mM (NH₄)₂SO₄, 20 mM MgSO₄)

Amplification is carried out as follows:

| | | | Number of |
|---|---|---|---|
| Cycling conditions | Temp. (° C.) | Time (min.) | cycles |
| Initial denaturing | 96 | 4 | 1 |
| step | | | |
| Denaturing step | 94 | 0.5 | 20 |
| Annealing step | 50 | 1 | 20 |
| Extension step | 72 | 1 | 20 |
| Final extension step | 72 | 5 | 1 |

1.C. Transformation and Selection of Transformants

A single PCR product of 522 basepairs is thus amplified and is then digested at 37° C. for 2 hours with SacI and XhoI concurrently in a 20 μl reaction volume. The digested product is ligated to similarly cleaved pET28a (Novagen) that is dephosphorylated prior to the ligation by treatment with Calf Intestinal Alkaline Phosphatase (CIP). The gene fusion constructed in this manner allows one-step affinity purification of the resulting fusion protein because of the presence of histidine residues at the N-terminus of the fusion protein, which are encoded by the vector.

The ligation reaction (20 μl) is carried out at 14° C. overnight and then is used to transform 100 μl fresh E. coli XL1-blue competent cells (Novagen). The cells are incubated on ice for 2 hours, then heat-shocked at 42° C. for 30 seconds, and returned to ice for 90 seconds. The samples are then added to 1 ml LB broth in the absence of selection and grown at 37° C. for 2 hours. The cells are then plated out on LB agar plus kanamycin (50 μg/ml final concentration) at a 10× and neat dilution and incubated overnight at 37° C. The following day, 50 colonies are picked onto secondary plates and incubated at 37° C. overnight.

Five colonies are picked into 3 ml LB broth supplemented with kanamycin (100 μg/ml) and grown overnight at 37° C. Plasmid DNA is extracted using the Quiagen mini-prep. method and quantitated by agarose gel electrophoresis.

PCR is performed with the gene-specific primers under the conditions stated above and transformant DNA is confirmed to contain the desired insert.

If PCR-positive, one of the five plasmid DNA samples (500 ng) extracted from the E. coli XL1-blue cells is used to transform competent BL21 (IDE3) E. coli competent cells (Novagen; as described previously). Transformants (10) are picked onto selective kanamycin (50 μg/ml) containing LB agar plates and stored as a research stock in LB containing 50% glycerol.

1.D. Recombinant Production of the Protein

Frozen stock (10 μl) is plated onto selection plates and grown for single colonies overnight at 37° C. A few cells are harvested from the plate and used as the inoculum for an overnight starter culture (3 ml) at 37° C. The following day, a sample (time ‘t’=0) is collected and centrifuged at 14,000 rpm for 3 minutes (samples are standardized by OD₆₀₀ for each time-point). The supernatant is discarded and the cells are stored at −20° C. for SDS-PAGE. This allows detection of leaky expression in the absence of the inducer IPTG. The overnight starter culture is then used to inoculate LB medium containing kanamycin (100 μg/ml) at a dilution of 1:50 (starting OD₆₀₀=0.05−0.1). The cells are grown to an OD₆₀₀ of 1.0, a sample is harvested for SDS-PAGE (pre-induction sample), and the remaining culture is induced with 1 mM IPTG. The cultures are grown for 4 hours and samples are taken every hour.

The culture is spun in a centrifuge at 6000× g for 20 minutes at 4° C. The supernatant is discarded and the pellets are resuspended in 50 ml of cold 50 mM Tris-HCl (pH 8.0), 2 mM EDTA, and spun as is described above. The supernatant is discarded and the cells are stored at −70° C.

1.E. Protein Purification

Pellets obtained from a 1 liter culture prepared as described in 1.D. are thawed and resuspended in 20 ml of ice cold 20 mM Tris-HCl (pH 8.0), 0.5 M NaCl, 5 mM Imidazole. Lysozyme is added to a concentration of 0.1 mg/ml and the suspension is homogenized using a high speed homogenizer (Turrax), and subsequently is treated in a sonicator (Branson, Sonifier 450). To remove DNA, Benzonase (Merck) is used at a final concentration of 1 U/ml. The suspension is centrifuged at 40,000× g for 20 minutes and the supernatant is filtered through a 0.45 μm membrane. The supernatant is loaded onto an IMAC column (12 ml of resin) that has been prepared by immobilizing Ni⁺⁺ according to the recommendations of the manufacturer (Pharmacia). The column is washed with 10 column volumes of 20 mM Tris-HCl (pH 8.0), 0.5 M NaCl, 60 mM Imidazole. The recombinant protein is eluted with 6 volumes of 20 mM Tris-HCl (pH 7.9), 0.5 M NaCl, 500 mM Imidazole, 0.1% Zwittergent 3-14.

The elution profile is monitored by measuring the absorbance of the fractions at OD 280 nm. An aliquot of each fraction is analyzed on SDS-PAGE gels and stained with Coomassie blue (Phast System—Pharmacia), and the fractions corresponding to the protein peak are then pooled and concentrated. To remove elution buffer, the fraction is passed over a G25 Sephadex column (Pharmacia), equilibrated in PBS (pH 7.4). The protein solution is filter-sterilized through a 0.45 μm membrane, and the protein concentration is determined by the BCA micromethod (Pierce). The protein solution is stored at −70° C.

1.F. Evaluation of the Protective Activity of the Purified Protein

Groups of 8 Swiss-Webster mice (Taconic) are immunized orally with 25 μg of the purified recombinant protein, admixed with 5 μg of cholera toxin (Calbiochem) in physiological buffer. Mice are immunized on days 0, 7, 14, and 21. Fourteen days after the last immunization, the mice are challenged with H. pylori strain ORV2001 grown in liquid media (the cells are grown on agar plates as described in 1.1. and, after harvest, the cells are resuspended in Brucella broth; the flasks are incubated overnight at 37° C.). Fourteen days after challenge, the mice are sacrificed and their stomachs are removed. The amount of H. pylori is determined by measuring the urease activity in the stomach and by culture.

1.G. Production of Monospecific Polyclonal Antibodies

1.G.1. Hyperimmune Rabbit Antiserum

New Zealand rabbits are injected both subcutaneously and intramuscularly with 100 μg (in total) of the purified fusion polypeptide as obtained in 1.E., in the presence of Freund's complete adjuvant in a total volume of approximately 2 ml. Twenty-one and 42 days after the initial injection, booster doses, which are identical to priming doses, except that Freund's incomplete adjuvant is used, are administered in the same way. Fifteen days after the last injection, animal serum is recovered, decomplemented, and filtered through a 0.45 μm membrane.

1.G.2. Mouse Hyperimmune Ascitic Fluid

Ten mice are injected subcutaneously with 10-50 μg of the purified fusion polypeptide as obtained in 1.E., in the presence of Freund's complete adjuvant in a volume of approximately 200 μl. 7 and 14 days after the initial injection, booster doses, which are identical to priming doses, except that Freund's incomplete adjuvant is used, are administered in the same way. 21 and 28 days after the initial infection, mice receive 50 μg of the antigen alone intraperitoneally. On day 21, mice are also injected intraperitoneally with sarcoma 180/TG cells CM26684 (Lennette et al., Diagnostic procedures for viral, rickettsial, and chlamydial infections, (1979) 5^{th} Ed. Washington D.C., American Public Health Association). Ascites are collected 10-13 days after the last injection.

1.H. Purification by Immunoaffinity

1.H.1. Purification of Specific IgGs

An immune serum as prepared in section 1.G. is applied to a protein A Sepharose 4 Fast Flow column (Pharmacia) equilibrated in 100 mM Tris-HCl (pH 8.0). The resin is washed by applying 10 column volumes of 100 mM Tris-HCl and 10 volumes of 10 mM Tris-HCl (pH 8.0) to the column. IgGs are eluted with a 0.1 M glycine buffer (pH 3.0) and are collected as 5 ml fractions to which is added 0.25 ml 1 M Tris-HCl (pH 8.0). The optical density of the eluate is measured at 280 nm and the fractions containing the IgGs are pooled, and, if necessary, stored frozen at −70° C.

1.H.2. Preparation of the Column

An appropriate amount of CNBr-activated Sepharose 4B gel (1 g of dried gel provides for approximately 3.5 ml of hydrated gel; gel capacity is of from 5 to 10 mg coupled IgGs per ml of gel) manufactured by Pharmacia (17-0430-01) is suspended in 1 mM HCl buffer and washed with a buchner by adding small quantities of 1 mM HCl buffer. The total volume of buffer is 200 ml per gram of gel.

Purified IgGs are dialyzed for 4 hours at 20±5° C. against 50 volumes of 500 mM sodium phosphate buffer (pH 7.5). Then they are diluted in 500 mM phosphate buffer (pH 7.5) to a final concentration of 3 mg/ml.

IgGs are incubated with the gel overnight at 5±3° C., under stirring. The gel is packed into a chromatography column and washed with 2 column volumes of 500 mM phosphate buffer (pH 7.5), then 1 volume of 50 mM sodium phosphate buffer, 500 mM NaCl (pH 7.5). The gel is then transferred to a tube and further incubated in 100 mM ethanolamine, (pH 7.5) for 4 hours at room temperature under stirring, then washed twice with 2 column volumes of PBS. The gel is then stored in 1/10,000 PBS merthiolate. The amount of IgGs coupled to the gel is determined by measuring the optical density (OD) at 280 nm of the IgG solution and the direct eluate, plus washings.

1.H.3. Adsorption and Elution of the Antigen

An antigen solution in 50 mM Tris-HCl (pH 8.0), 2 mM EDTA, for example, the supernatant obtained in 1.E. after the Benzonase treatment, centrifugation, and filtration through a 0.45 μm membrane, is applied to a column equilibrated with 50 mM Tris-HCl (pH 8.0), 2 mM EDTA, at a flow rate of about 10 ml/hour. Then the column is washed with 20 volumes 50 mM Tris-HCl (pH 8.0), 2 mM EDTA. Alternatively, adsorption can be achieved in a batch that is let to stand overnight at 5±3° C., under stirring.

The gel is washed with 2 to 6 volumes of 10 mM sodium phosphate buffer (pH 6.8). The antigen is eluted with 100 mM glycine buffer (pH 2.5). The eluate is recovered in 3 ml fractions to which is added 150 μl 1 M sodium phosphate buffer (pH 8.0). OD is measured at 280 nm for each fraction; those containing the antigen are pooled and stored at −20° C.

### EXAMPLE 2

### Preparation of Isolated DNA Encoding the Polypeptides of the Invention from the Deposited Clones.

As mentioned above, E. coli strains including plasmids containing nucleic acids encoding HPO76 (98197), HPO18 (98210), HPO121(98201), HPO45 (98208), HPO101(98198), HPO116 (98200), HPO7 (98211), HPO104 (98199), HPO15 (98214), HPO58 (98206), HPO132 (98202), HPO9 (98203), HPO38 (98204), HPO87 (98205), HPO71(98217), HPO70 (98219), HPO80 (98215), HPO95 (98216), HPO98 (98218), HPO57 (98220), HPO50 (98207), HPO64 (98213), HPO54 (98212), and HPO42 (98209) were deposited in E. coli strain DH5α under the Budapest Treaty with the American Type Culture Collection (ATCC; Rockville, Md.) on Oct. 9, 1996 and were designated with accession numbers indicated in parentheses above. These plasmids each contain a genomic DNA BglII-ClaI insert from H. pylori strain P1 or P12 (referred to as 69-A and 888-0 in Haas et al., Mol. Microbiol. 8:753, 1993). Each of the inserts are disrupted by the presence of transposon TnMax9 (Kahrs et al., Gene 167:53, 1995). DNA molecules lacking the transposon can be amplified from the plasmids using standard PCR techniques, such as inverse and recombinant PCR (see, e.g., Innis et al., supra), so that a full-length H. pylori insert is reconstituted. For example, the H. pylori sequences flanking the transposon can each be amplified by PCR, and then ligated together to form the full-length H. pylori gene lacking the transposon. Primers that can be used in these methods for each of the twenty-four clones of the invention are shown in Table 1.

### EXAMPLE 3

### Purification of Recombinant H. Pylori Antigen from Clone 76 (HPO76)

A pellet of E. coli expressing HPO76 is homogenized in 5 mM imidazole, 500 mM sodium chloride, 20 mM Tris-HCl (pH 7.9) by microfluidization at a pressure of 15,000 psi, and clarified by centrifugation at 4000-5000 g.

Method 1

The pellet containing cloned protein is suspended in buffer containing 2% N-octyl glucoside (NOG) and is homogenized. The NOG soluble protein is removed by centrifugation. The pellet is extracted one more time with 2% NOG. After centrifugation, the pellet is dissolved in 8 M urea. The urea-solubilized protein is diluted with an equal volume of 2 M arginine and dialyzed against 1 M arginine for 24-48 hours to remove urea. The cloned protein remains in solution. SDS-PAGE and Coomassie staining, followed by densitometric scanning, shows that the protein is 80-85% pure cloned antigen.

Method 2

The pellet containing cloned protein is solubilized in 6 M guanidine hydrochloride and is passed through an IMAC column charged with Ni⁺⁺. The bound antigen is eluted with 8 M urea (pH 8.5). β-mercaptoethanol is added to eluted protein to a final concentration of 1 mM, then passed through a Sephadex G-25 column equilibrated in 0.1 M acetic acid. Protein eluted from Sephadex G-25 column is slowly added to 4 volumes of 50 mM phosphate (pH 7.0). The protein remains in solution.

Purification of Recombinant Proteins

Recombinant proteins expressed as Histidine-tagged fusion proteins can be solubilized and purified by using a metal affinity column (nickel column). The bound protein can be eluted with imidazole buffer, with or without urea, or by using low pH buffers, with or without urea. Urea or guanidine hydrochloride-denatured proteins can then be renatured using appropriate renaturing buffers. With a number of recombinant H. pylori antigens (HpaA and clone 76), renaturation conditions using arginine hydrochloride (0.25-1 M) have been determined.

Recombinant proteins without a His-tag can be solubilized and purified using immunoaffinity, ion-exchange, sizing, and/or hydrophobic chromatography. Proteins expressed as insoluble aggregates in inclusion bodies can be solubilized in denaturing agents, such as 8 M urea or 6 M guanidine hydrochloride. Appropriate folding and renaturation can readily be determined by one skilled in the art.

The above pellet containing cloned protein is suspended in 50 mM NaPO₄ (pH 7.5) containing 1% weight/volume N-octyl glucoside (NOG) and mixed vigorously. The NOG soluble impurities are removed by centrifugation. The remaining pellet is extracted one more time with the 1% NOG solution to further remove impurities. After centrifugation, the pellet is solubilized in 8 M urea, 50 mM Tris (pH 8.0). The Urea solubilized protein is diluted with an equal volume of 2 M Arginine, 50 mM Tris (pH 8.0), and is dialyzed against 1 M Arginine, 50 mM Tris, 50 mM NaCl (pH 8.0) for 24-48 hours to remove urea. The cloned protein remains in solution following dialysis. SDS-PAGE and Coomassie staining followed by densitometric scanning shows that the protein is 80-85% pure cloned antigen.

### EXAMPLE 4

### Method for Production of Transcriptional Fusions Lacking His-Tags

Methods for amplification and cloning of DNA encoding HPO76 as a transcriptional fusion lacking His-tags are described as follows. These methods can readily be adapted by one skilled in the art for similar amplification and cloning of DNA encoding the other polypeptides of the invention.

Amplification of Clone 76 DNA

Design of PCR Primers for Cloning

Two PCR primers are designed based on the complete gene sequence (see table 1).

The N-terminal primer (FC1) is designed to include the ribosome binding site of the target gene (underlined), the ATG start site (bold), and the leader sequence (with cleavage site). It includes a clamp (GCC) at the 5′ most end, and a SacI recognition sequence (GAGCTC) for cloning purposes.

The C-terminal primer (RN2) includes an XhoI recognition sequence for cloning purposes, and the natural TAA stop codon (bold).

| | | |
|---|---|---|
| N-terminal primer (FC1) | 5′GCC[GAGCTC]CAAGCAAAAAAATGTCAATTAAAAGGG3′ | (SEQ ID NO:) |
| C-terminal primer (RN2) | 5′GCC[CTCGAG]GTCTAAATTAGAATAAGTGTTGTT 3′ | (SEQ ID NO:) |

Amplification of each specified gene can be achieved by employing FC1/RN2 primers for any of the genes described (see Table 1).

PCR Conditions

Amplification of gene-specific DNA is carried out using Pwo DNA Polymerase (Boehringer Mannheim) under the following conditions. Due to the exonuclease activity of the polymerase, two reaction mixtures are prepared separately and combined just prior to amplification.

Reaction ingredients:

| Ingredient (final conc.) | Mixture 1 (μl) | Mixture 2 (μl) |
|---|---|---|
| distilled H₂O | 160 | 79 |
| dNTPs (200 μM each) | 40 | — |
| 10X buffer | — | 20 |
| primer 1 (100 nM) | 1 | — |
| primer 2 (100 nM) | 1 | — |
| Template (200 ng) | 2 | 0 |

| Cycling condition | Temp (° C.) | Time (min.) | Number of cycles |
|---|---|---|---|
| Initial denaturing step | 96 | 4 | 1 |
| Denaturing step | 94 | 0.5 | 20 |
| Annealing step | 50 | 1 | 20 |
| Extension step | 72 | 1 | 20 |
| Final extension step | 72 | 1 | 1 |

A single PCR product of 624 basepairs is amplified and cloned into SacI-XhoI cleaved pET 24, allowing construction of a transcriptional fusion and expression of HPO76 antigen in the absence of a His-tag. In this instance, expressed product can be purified as a denatured protein that is re-folded by dialysis into 1 M arginine.

Cloning into pET 24 allows transcription from the T7 promoter, supplied by the vector, but relies upon binding of the RNA-specific DNA polymerase to the intrinsic ribosome binding site for HPO76, and thereby expression of the complete ORF. The amplification, restriction, and cloning protocols are as previously described for constructing translational fusions.

TABLE 1
RE-CONSTRUCTION OF A COMPLETE ORF BY RECOMBINANT PCR

F′ denotes forward primer
R′ denotes reverse primer
C′ denotes coding strand
N′ denotes non-coding strand
Alt FC1 and RN2 primers have incorporated at their 5′ end a clamp and a recognition sequence
for cloning purposes
GGC clamp present for amplification and cloning of entire gene sequence from chromosomal DNA
[X] denotes any nucleotide sequence not present in the completed gene sequence
() Identifies region of overlap between the two original PCR products, and is consistently 10
nucleotides long for each clone

| | | | | Length | |
|---|---|---|---|---|---|
| CLONE No. | Prtmer type | nt positions | Primer sequence (5′-3′) | of gene seq. | Tm (oC) |
| 76 | FC1 | 304-330 | GCC[x] CAAGCAAAAAAATGTCAATTAAAAGGG | 27 | 70 |
| | RN1 | 413-391 | TAAGTCCATACGATAGCCTATG | 22 | 62 |
| | FC2 | 404-436 | (TATGGAACTTA) GAACATTTTAACACGCTCTATTA | 33 | 60 |
| | RN2 | 927-904 | GCC [X] GTCTAAATTAGAATAAGTGTTGTT | 24 | 60 |
| 18 | FC1 | 101-124 | GCC[X] AATATATGGGAACTTAATGAGAAT | 24 | 60 |
| | RN1 | 227-206 | TGCGAGATTTAACCTGTTTTCA | 22 | 60 |
| | FC2 | 218-249 | (AAATCTCGCA) GAAATCTTTCACAAGCGAGCAA | 32 | 60 |
| | RN2 | 922-901 | GCC [X] ATGTCATGTCAAACTATGAAGC | 22 | 60 |
| 121 | FC1 | 141-164 | GCC [X] TCACAATGGATAAAAACAACAACA | 24 | 62 |
| | RN1 | 451-473 | GCCCTTTTGTTTAGGGGTTAG | 21 | 62 |
| | FC2 | 455-485 | (ACAAAAGGGC) TTTTTAGAGCATGTGAGCCATC | 32 | 62 |
| | RN2 | 814-796 | GCC [X] CTGTCCAAATCAGCCACCC | 19 | 60 |
| 45 | FC1 | 1-26 | GCC [X] ATGAAAAGATTTGATTTGTTTTTATC | 26 | 62 |
| | RN1 | 299-278 | AAGCCGTATTGTTTGTTTTGGC | 22 | 62 |
| | FC2 | 290-323 | (AATACGGCTTTAAAGCTATAGAAAATTTAAACGC) | 34 | 60 |
| | RN2 | 603-582 | GCC[X] TTAAATATCCCAATCCTGCCAC | 22 | 62 |
| 101 | FC1 | 308-332 | GCC[X] GAAGGATTTATTATGATTAAAAGAA | 25 | 60 |
| | RN1 | 497-474 | AACCTAATTTGAAATTCAAACCAT | 24 | 60 |
| | FC2 | 488-519 | (AAATTAGGTT) TTGTAGGCTTTGCCAATAAATG | 32 | 60 |
| | RN2 | 893-869 | GCC[X] AAGGAATAAATTAGAAAGTGAAGAA | 25 | 62 |
| 116 | FC1 | 236-259 | GCC [X] CGCATTGATTTGATGAATAAACC | 23 | 62 |
| | RN1 | 434-416 | CGCCTATAACCGCTCCATT | 19 | 60 |
| | FC2 | 425-456 | (GTTATAGGCG) ATAAAGGTTTAACGCAGCTAAG | 32 | 60 |
| | RN2 | 812-790 | GCC [X] CTCACTAAAAAGCAATTTTTGAG | 23 | 60 |
| 7 | FC1 | 195-220 | GCC [X] TAAGGAATGAAGTTGATAAAATTTGT | 26 | 64 |
| | RN1 | 349-327 | GCATTTTCATTCATTCTTTGGAC | 23 | 60 |
| | FC2 | 339-371 | (ATGAAAATGC) ACGCCCAAATAATAAGGAAGTA | 32 | 60 |
| | RN2 | 738-717 | GCC [X] GGATTTATTGAGCTTTCCCCTT | 22 | 62 |
| 104 | FC1 | 251-271 | GCC [X] AAAGGGCGAAAATGAGCAAGA | 21 | 60 |
| | RN1 | 429-407 | TAAAATAACCAACAGAGTGATCA | 23 | 60 |
| | FC2 | 420-452 | (GGTTATTTTA) GTGGATATTTGGGTTTATAGCGA | 33 | 62 |
| | RN2 | 784-761 | GCC [X] TTTTTTAAGAATCACTTTCTTCGG | 24 | 62 |
| 58 | GC1 | 118-143 | GCC [X] ATAGGAACAAGCATGTTTTTTAAAAC | 26 | 66 |
| | RN1 | 434-413 | TGAAGTCTTGCGATTTTTGCTT | 22 | 60 |
| | FC2 | 425-454 | (CAAGACTTCA) AAAAAGAAGGAGCGGTTGCC | 30 | 60 |
| | RN2 | 650-630 | GCC [X] CTGGCTTATTGCGTATCATC | 20 | 60 |
| 132 | FC1 | 294-314 | GGC [X] GGAAGAATAATGCTCGCTTCC | 21 | 62 |
| | RN1 | 409-378 | ACTGGAGTGTGGATAAAACTAT | 22 | 60 |
| | FC2 | 400-430 | (ACACTCCAGT) AGATGCTTTCCCGGATATTTC | 31 | 60 |
| | RN2 | 761-741 | GCC [X] CTATTCTCCAGGGATATGGCC | 21 | 64 |
| 9 | FC1 | 211-233 | GCC [X] GATGGATTTTTTATGGGGGTGAG | 23 | 64 |
| | RN1 | 347-328 | GGCACTGCCGCAGATTCTA | 19 | 60 |
| | FC2 | 338-370 | (CGGCAGTGCC) TTTAGCCTATTATTTAGAAGCGA | 33 | 60 |
| | RN2 | 686-665 | GCC [X] ATGGTATTTGTCTAAGACCCTC | 22 | 62 |
| 38 | FC1 | 220-242 | GCC [X] AAAAGGGTTTTAAATAATGGCTG | 23 | 60 |
| | RN1 | 348-327 | ACAAGGATAAAAAACGCGCTAA | 22 | 60 |
| | FC2 | 239-371 | (TTATCCTTGT) TGCTGGCTTGGTTTTTTTTAATT | 33 | 60 |
| | RN2 | 597-575 | GCC [X] AAGATTCTAAAAGGGCTTCAAAT | 23 | 60 |
| 71 | FC1 | 1-25 | GCC [X] ATGTTGAAATTTAAATATGGTTTGA | 25 | 60 |
| | RN1 | 274-254 | AAACCCCACTCTTATCATCGG | 21 | 62 |
| | FC2 | 265-294 | (AGTGGGGTTT) TTTTAGGGGGTGGGTATGCT | 30 | 60 |
| | RN2 | 524-505 | GCC [X] GAGCCTACAGGTTGCTTGC | 20 | 60 |
| 70 | FC1 | 1-23 | GCC [X] ATGGTATTTGACAGAACAATCAG | 23 | 62 |
| | RN1 | 115-96 | GAAAAGCCACCCCGCTTATT | 20 | 60 |
| | FC2 | 106-137 | (GTGGCTTTTC) AAAAAGAGTGGGTGCAACAATT | 32 | 60 |
| | RN2 | 495-471 | GCC [X] TTAGGAATAGCATAACAAACAAACG | 25 | 66 |
| 80 | FC1 | 1-25 | GCC [X] ATGTTAGAAAAATTGATTGAAAGAG | 25 | 62 |
| | RN1 | 106-95 | TGAACACATAGCCTAAAACCAC | 21 | 62 |
| | FC2 | 97-127 | (TATGTGTTCA) TGAAAGAGTTGTGGCACATGC | 31 | 62 |
| | RN2 | 435-415 | GCC [X] TTATGCGATAGGGGGCGTATC | 21 | 66 |
| 95 | FC1 | 1-27 | GCC [X] ATGAAAAAATTTTTTTCTCAATCTTT | 27 | 60 |
| | RN1 | 64-46 | TGGCCAGTAGCGCGTTCAT | 19 | 60 |
| | FC2 | 55-98 | (CTACTGGCCA) TGGATGGCAATGGCGTTTTTTTAG | 34 | 68 |
| | RN2 | 432-408 | GCC [X] TTATTGATGAACATTAACCATTAAA | 25 | 60 |
| 98 | FC1 | 1-22 | GCC [X] ATGAAAACCTTTAAAAACCTGC | 22 | 58 |
| | RN1 | 43-23 | TAGCGATCAGGCTAAAACAGA | 21 | 60 |
| | FC2 | 34-62 | (CTGATCGCTA) TGAGTTGGCTCCAAGCGGA | 29 | 60 |
| | RN2 | 336-313 | GCC [X] TTAAAACTCATAGCGTTTTTCAAT | 24 | 60 |
| 42 | FC1 | 18-51 | GCC [X] GAGAGTAGTGGCAGAGTTTATGCTGATTCCC | 34 | 98 |
| | RN1 | 380-351 | (AACTTTTC)TCTATCCCAATTCGTTACGCTC | 30 | 64 |
| | FC2 | 366-396 | (GGATAGA)GAAAAGTTTGGCGTCAAAAGTTGG | 31 | 68 |
| | RN2 | 822-801 | GCC [X] GGCTTAAACTGGAACGGATTTC | 22 | 64 |
| 50 | FC1 | 140-170 | GCC [X] TAAAGTTTGCTAAAAAGATGGTTTTAATTTC | 31 | 76 |
| | RN1 | 297-270 | (GACTTCTAAAG)CGTCCTTTTTTTCTTTA | 28 | 56 |
| | FC2 | 287-314 | (CTTTA)GAAGTCATTAAACAAAGAGGGGT | 29 | 64 |
| | RN2 | 607-584 | GCC [X] CCCATCTTTAGAAATCAACCCCCA | 24 | 70 |
| 64 | FC1 | 23-50 | GCC [X] GAAATAAGGAGTTTGTATGCAACAGCG | 28 | 80 |
| | RN1 | 225-149 | (A)AGCTTTTCATTATCTTCCCCATAAGC | 27 | 74 |
| | FC2 | 216-244 | (TGAAAAGCT)TTTAGCGAAGCGATCAAGCC | 29 | 60 |
| | RN2 | 1039-1012 | GCC [X] CCCAATACTTTTATTGATTCACCATTTC | 28 | 74 |
| 54 | FC1 | 21-48 | GCC [X] CAATAAAACACCAAAATGAATGAGTTAC | 28 | 68 |
| | RN1 | 352-327 | (A)GATTTTGTTTTGAGCGTTAGAAATG | 26 | 66 |
| | FC2 | 345-376 | (CAAAATC)TATAAACTCAATCAAGTCAAAAATG | 32 | 62 |
| | RN2 | 1280-1255 | GCC [X] GCATTTACCCCCTAAAAACTATAAAC | 26 | 70 |
| 15 | FC1 | 14-35 | GCC [X] CTGAAGGGTGTATGGTATTAGG | 22 | 64 |
| | RN1 | 157-132 | (C)ACCATACATGTATCCTGCATTAATG | 26 | 68 |
| | FC2 | 147-179 | (CATGTATGGT)GTAGCAAAGAATTTTAAGGAGGC | 33 | 64 |
| | RN2 | 377-349 | GCC [X] CGTTAAAACTAAAGTTCTATTTTTAATTC | 29 | 70 |
| 57 | FC1 | 13-39 | GCC [X] GTAAGGAATGAGATGATAAAGAGTTGG | 27 | 74 |
| | RN1 | 267-244 | (T)GGAATATTCTGATCCACGCCATC | 24 | 68 |
| | FC2 | 258-294 | (GAATATTCC)AAAAGCCGTTTTTTATTACAGAAGAGC | 37 | 76 |
| | RN2 | 957-934 | GCC [X] CTAAACTCTGGCTTATTGCGTATC | 24 | 68 |
| 87 | FC1 | 1-22 | GCC [X] ATGCGTTTATTATTGTGGTGGG | 22 | 62 |
| | RN1 | 27-3 | (C)AATACCCACCACAATAATAAACGCAT | 25 | 66 |
| | FC2 | 18-50 | (GTGGGTATT)GGTATTATCGCTCTTTTTAAATCC | 33 | 64 |
| | RN2 | 519-498 | GCC [X] TTAAATTTTTAGGGAAAGGGTA | 22 | 62 |

CONDITIONS FOR RECOMBINANT PCR
Two independent PCR reactions are carried out for FC1/RN1 and fC2/RN2 primers under the same
conditions proposed for cloning genes for expression.
After 20 cycles, the product of each reaction is used as template for a further 20 cycles with
FC1/RN2 only
The product will encompass the full length gene minus the transposon.
The presence of restriction sites at the 5′ ends of these primers allows for cloning/expression
studies.

What is claimed is:

1. An isolated polynucleotide that encodes (i) a polypeptide comprising an amino acid sequence that is homologous to the amino acid sequence of a Helicobacter membrane-associated polypeptide, wherein said amino acid sequence of said Helicobacter membrane-associated polypeptide is selected from the group consisting of:
(a) the amino acid sequences as shown:
in SEQ ID NO:2, beginning with an amino acid in any one of the positions from −27 to 5, and ending with an amino acid in position 160 (HPO101);
in SEQ ID NO:4, beginning with an amino acid in position 1 and ending with an amino acid in position 172 (HPO104);
in SEQ ID NO:6, beginning with an amino acid in any one of the positions from −17 to 5, and ending with an amino acid in position 169 (HPO116);
in SEQ ID NO:8, beginning with an amino acid in any one of the positions from −21 to 5, and ending with an amino acid in position 198 (HPO121);
in SEQ ID NO:10, beginning with an amino acid in any one of the positions from −20 to 5, and ending with an amino acid in position 132 (HPO132);
in SEQ ID NO:12, beginning with an amino acid in positions 1 and ending with an amino acid in position 114 (HPO15);
in SEQ ID NO:14, beginning with an amino acid in any one of the positions from −17 to 5, and ending with an amino acid in position 248 (HPO18);
in SEQ ID NO:16, beginning with an amino acid in any one of the positions from −40 to 5, and ending with an amino acid in position 74 (HPO38);
in SEQ ID NO:18, beginning with an amino acid in any one of the positions from −34 to 5, and ending with an amino acid in position 226 (HPO42);
in SEQ ID NO:20, beginning with an amino acid in any one of the positions from −21 to 5, and ending with an amino acid in position 179 (HPO45);
in SEQ ID NO:22, beginning with an amino acid in any one of the positions from −33 to 5, and ending with an amino acid in position 114 (HPO50);
in SEQ ID NO:24, beginning with an amino acid in any one of the positions from −60 to 5, and ending with an amino acid in position 349 (HPO54);
in SEQ ID NO:26, beginning with an amino acid in any one of the positions from −18 to 5, and ending with an amino acid in position 288 (HPO57);
in SEQ ID NO:28, beginning with an amino acid in any one of the positions from −21 to 5, and ending with an amino acid in position 150 (HPO58);
in SEQ ID NO:30, beginning with an amino acid in any one of the positions from −20 to 5, and ending with an amino acid in position 309 (HPO64);
in SEQ ID NO:32, beginning with an amino acid in any one of the positions from −35 to 5, and ending with an amino acid in position 129 (HPO70);
in SEQ ID NO:34, beginning with an amino acid in any one of the positions from −19 to 5, and ending with an amino acid in position 153 (HPO71);
in SEQ ID NO:36, beginning with an amino acid in any one of the positions from −25 to 5, and ending with an amino acid in position 176 (HPO76);
in SEQ ID NO:38, beginning with an amino acid in any one of the positions from −21 to 5, and ending with an amino acid in position 156 (HPO7);
in SEQ ID NO:40, beginning with an amino acid in position 1 and ending with an amino acid in position 144 (HPO80);
in SEQ ID NO:42, beginning with an amino acid in any one of the positions from −20 to 5, and ending with an amino acid in position 152 (HPO87);
in SEQ ID NO:44, beginning with an amino acid in any one of the positions from −31 to 5, and ending with an amino acid in position 112 (HPO95);
in SEQ ID NO:46, beginning with an amino acid in any one of the positions from −20 to 5, and ending with an amino acid in position 91 (HPO98);
in SEQ ID NO:48, beginning with an amino acid in any one of the positions from −21 to 5, and ending with an amino acid in position 129 (HPO9); and
(b) the precursor or mature amino acid sequences encoded by the Helicobacter DNA inserts found in American Type Culture Collection deposit numbers 98197 (HPO76), 98210 (HPO18), 98201 (HPO121), 98208 (HPO45), 98198 (HPO101), 98200 (HPO116), 98211 (HPO7), 98199 (HPO104), 98214 (HPO15), 98206 (HPO58), 98202 (HPO132), 98203 (HPO9), 98204 (HPO38), 98205 (HPO87), 98217 (HPO71), 98219 (HPO70), 98215 (HPO80), 98216 (HPO95), 98218 (HPO98), 98220 (HPO57), 98207 (HPO50), 98213 (HPO64), 98212 (HPO54), and 98209 (HPO42); or (ii) a derivative of said polypeptide encoded by said polynucleotide.

2. An isolated polynucleotide that encodes (i) a polypeptide comprising an amino acid sequence that is homologous to an amino acid sequence selected from the group consisting of:
(a) the amino acid sequences as shown:
in SEQ ID NO:2, beginning with an amino acid in position −27 and ending with an amino acid in position 160 (HPO101);
in SEQ ID NO:4, beginning with an amino acid in position 1 and ending with an amino acid in position 172 (HPO104);
in SEQ ID NO:6, beginning with an amino acid in position −17 and ending with an amino acid in position 169 (HPO116);
in SEQ ID NO:8, beginning with an amino acid in position −21 and ending with an amino acid in position 198 (HPO121);
in SEQ ID NO:10, beginning with an amino acid in position −20, and ending with an amino acid in position 132 (HPO132);
in SEQ ID NO:12, beginning with an amino acid in position 1 and ending with an amino acid in position 114 (HPO15);
in SEQ ID NO:14, beginning with an amino acid in position −17 and ending with an amino acid in position 248 (HPO18);
in SEQ ID NO:16, beginning with an amino acid in position −40 and ending with an amino acid in position 74 (HPO38);
in SEQ ID NO:18, beginning with an amino acid in position −34 and ending with an amino acid in position 226 (HPO42);
in SEQ ID NO:20, beginning with an amino acid in position −21 and ending with an amino acid in position 179 (HPO45);
in SEQ ID NO:22, beginning with an amino acid in position −33 and ending with an amino acid in position 114 (HPO50);
in SEQ ID NO:24, beginning with an amino acid in position −60 and ending with an amino acid in position 349 (HPO54);
in SEQ ID NO:26, beginning with an amino acid in position −18 and ending with an amino acid in position 288 (HPO57);
in SEQ ID NO:28, beginning with an amino acid in position −21 and ending with an amino acid in position 150 (HPO58);
in SEQ ID NO:30, beginning with an amino acid in position −20 and ending with an amino acid in position 309 (HPO64);
in SEQ ID NO:32, beginning with an amino acid in position −35 and ending with an amino acid in position 129 (HPO70);
in SEQ ID NO:34, beginning with an amino acid in position −19 and ending with an amino acid in position 153 (HPO71);
in SEQ ID NO:36, beginning with an amino acid in position −25 and ending with an amino acid in position 176 (HPO76);
in SEQ ID NO:38, beginning with an amino acid in position −21 and ending with an amino acid in position 156 (HPO7);
in SEQ ID NO:40, beginning with an amino acid in position 1 and ending with an amino acid in position 144 (HPO80);
in SEQ ID NO:42, beginning with an amino acid in position −20 and ending with an amino acid in position 152 (HPO87);
in SEQ ID NO:44, beginning with an amino acid in position −31 and ending with an amino acid in position 112 (HPO95);
in SEQ ID NO:46, beginning with an amino acid in position −20 and ending with an amino acid in position 91 (HPO98);
in SEQ ID NO:48, beginning with an amino acid in position −21 and ending with an amino acid in position 129 (HPO9); and
(b) the amino acid sequences encoded by the DNA inserts found in American Type Culture Collection deposit numbers 98197 (HPO76), 98210 (HPO18), 98201 (HPO121), 98208 (HPO45), 98198 (HPO101), 98200 (HPO116), 98211 (HPO7), 98199 (HPO104), 98214 (HPO15), 98206 (HPO58), 98202 (HPO132), 98203 (HPO9), 98204 (HPO38), 98205 (HPO87), 98217 (HPO71), 98219 (HPO70), 98215 (HPO80), 98216 (HPO95), 98218 (HPO98), 98220 (HPO57), 98207 (HPO50), 98213 (HPO64), 98212 (HPO54), and 98209 (HPO42); or (ii) a derivative of said polypeptide.

3. The isolated polynucleotide of claim 1, which encodes the mature form of (i) a polypeptide comprising an amino acid sequence that is homologous to an amino acid sequence selected from the group consisting of:
(a) the amino acid sequences as shown:
in SEQ ID NO:2, beginning with an amino acid in position −27 and ending with an amino acid in position 160 (HPO101);
in SEQ ID NO:4, beginning with an amino acid in position 1 and ending with an amino acid in position 172 (HPO104);
in SEQ ID NO:6, beginning with an amino acid in position −17 and ending with an amino acid in position 169 (HPO116);
in SEQ ID NO:8, beginning with an amino acid in position −21 and ending with an amino acid in position 198 (HPO121);
in SEQ ID NO:10, beginning with an amino acid in position −20, and ending with an amino acid in position 132 (HPO132);
in SEQ ID NO:12, beginning with an amino acid in position 1 and ending with an amino acid in position 114 (HPO15);
in SEQ ID NO:14, beginning with an amino acid in position −17 and ending with an amino acid in position 248 (HPO18);
in SEQ ID NO:16, beginning with an amino acid in position −40 and ending with an amino acid in position 74 (HPO38);
in SEQ ID NO:18, beginning with an amino acid in position −34 and ending with an amino acid in position 229 (HPO42);
in SEQ ID NO:20, beginning with an amino acid in position −21 and ending with an amino acid in position 179 (HPO45);
in SEQ ID NO:22, beginning with an amino acid in position −33 and ending with an amino acid in position 114 (HPO50);
in SEQ ID NO:24, beginning with an amino acid in position −60 and ending with an amino acid in position 349 (HPO54);
in SEQ ID NO:26, beginning with an amino acid in position −18 and ending with an amino acid in position 288 (HPO57);
in SEQ ID NO:28, beginning with an amino acid in position −21 and ending with an amino acid in position 150 (HPO58);
in SEQ ID NO:30, beginning with an amino acid in position −20 and ending with an amino acid in position 309 (HPO64);
in SEQ ID NO:32, beginning with an amino acid in position −35 and ending with an amino acid in position 129 (HPO70);
in SEQ ID NO:34, beginning with an amino acid in position −19 and ending with an amino acid in position 153 (HPO71);
in SEQ ID NO:36, beginning with an amino acid in position −25 and ending with an amino acid in position 176 (HPO76);
in SEQ ID NO:38, beginning with an amino acid in position −21 and ending with an amino acid in position 156 (HPO7);
in SEQ ID NO:40, beginning with an amino acid in position 1 and ending with an amino acid in position 144 (HPO 80);
in SEQ ID NO:42, beginning with an amino acid in position −20 and ending with an amino acid in position 152 (HPO 87);
in SEQ ID NO:44, beginning with an amino acid in position −31 and ending with an amino acid in position 112 (HPO 95);
in SEQ ID NO:46, beginning with an amino acid in position −20 and ending with an amino acid in position 91 (HPO 98);
in SEQ ID NO:48, beginning with an amino acid in position −21 and ending with an amino acid in position 129 (HPO 9); and
(b) the amino acid sequences encoded by the DNA inserts found in American Type Culture Collection deposit numbers 98197 (HPO76), 98210 (HPO18), 98201 (HPO121), 98208 (HPO45), 98198 (HPO101), 98200 (HPO116), 98211 (HPO7), 98199 (HPO104), 98214 (HPO15), 98206 (HPO58), 98202 (HPO132), 98203 (HPO9), 98204 (HPO38), 98205 (HPO87), 98217 (HPO71), 98219 (HPO70), 98215 (HPO80), 98216 (HPO95), 98218 (HPO98), 98220 (HPO57), 98207 (HPO50), 98213 (HPO64), 98212 (HPO54), and 98209 (HPO42); or (ii) a derivative of said polypeptide.

4. The isolated polynucleotide of claim 1, wherein the polynucleotide is a DNA molecule.

5. The isolated polynucleotide of claim 1, which is a DNA molecule that can be amplified and/or cloned by polymerase chain reaction from an Helicobacter genome, using either:
A 5′ oligonucleotide primer having a sequence as shown in SEQ ID NO:49 wherein N is a restriction site, and a 3′ oligonucleotide primer having a sequence in SEQ ID NO:50 wherein N is a restriction site;
A 5′ oligonucleotide primer having a sequence as shown in SEQ ID NO:51 wherein N is a restriction site, and a 3′ oligonucleotide primer having a sequence in SEQ ID NO:52 wherein N is a restriction site;
A 5′ oligonucleotide primer having a sequence as shown in SEQ ID NO:53 wherein N is a restriction site, and a 3′ oligonucleotide primer having a sequence in SEQ ID NO:54 wherein N is a restriction site;
A 5′ oligonucleotide primer having a sequence as shown in SEQ ID NO:55 wherein N is a restriction site, and a 3′ oligonucleotide primer having a sequence in SEQ ID NO:56 wherein N is a restriction site;
A 5′ oligonucleotide primer having a sequence as shown in SEQ ID NO:57 wherein N is a restriction site, and a 3′ oligonucleotide primer having a sequence in SEQ ID NO:58 wherein N is a restriction site;
A 5′ oligonucleotide primer having a sequence as shown in SEQ ID NO:59 wherein N is a restriction site, and a 3′ oligonucleotide primer having a sequence in SEQ ID NO:60 wherein N is a restriction site;
A 5′ oligonucleotide primer having a sequence as shown in SEQ ID NO:61 wherein N is a restriction site, and a 3′ oligonucleotide primer having a sequence in SEQ ID NO:62 wherein N is a restriction site;
A 5′ oligonucleotide primer having a sequence as shown in SEQ ID NO:63 wherein N is a restriction site, and a 3′ oligonucleotide primer having a sequence in SEQ ID NO:64 wherein N is a restriction site;
A 5′ oligonucleotide primer having a sequence as shown in SEQ ID NO:65 wherein N is a restriction site, and a 3′ oligonucleotide primer having a sequence in SEQ ID NO:66 wherein N is a restriction site;
A 5′ oligonucleotide primer having a sequence as shown in SEQ ID NO:67 wherein N is a restriction site, and a 3′ oligonucleotide primer having a sequence in SEQ ID NO:68 wherein N is a restriction site;
A 5′ oligonucleotide primer having a sequence as shown in SEQ ID NO:69 wherein N is a restriction site, and a 3′ oligonucleotide primer having a sequence in SEQ ID NO:70 wherein N is a restriction site;
A 5′ oligonucleotide primer having a sequence as shown in SEQ ID NO:71 wherein N is a restriction site, and a 3′ oligonucleotide primer having a sequence in SEQ ID NO:72 wherein N is a restriction site;
A 5′ oligonucleotide primer having a sequence as shown in SEQ ID NO:73 wherein N is a restriction site, and a 3′ oligonucleotide primer having a sequence in SEQ ID NO:74 wherein N is a restriction site;
A 5′ oligonucleotide primer having a sequence as shown in SEQ ID NO:75 wherein N is a restriction site, and a 3′ oligonucleotide primer having a sequence in SEQ ID NO:76 wherein N is a restriction site;
A 5′ oligonucleotide primer having a sequence as shown in SEQ ID NO:77 wherein N is a restriction site, and a 3′ oligonucleotide primer having a sequence in SEQ ID NO:78 wherein N is a restriction site;
A 5′ oligonucleotide primer having a sequence as shown in SEQ ID NO:79 wherein N is a restriction site, and a 3′ oligonucleotide primer having a sequence in SEQ ID NO:80 wherein N is a restriction site;
A 5′ oligonucleotide primer having a sequence as shown in SEQ ID NO:81 wherein N is a restriction site, and a 3′ oligonucleotide primer having a sequence in SEQ ID NO:82 wherein N is a restriction site;
A 5′ oligonucleotide primer having a sequence as shown in SEQ ID NO:83 wherein N is a restriction site, and a 3′ oligonucleotide primer having a sequence in SEQ ID NO:84 wherein N is a restriction site;
A 5′ oligonucleotide primer having a sequence as shown in SEQ ID NO:85 wherein N is a restriction site, and a 3′ oligonucleotide primer having a sequence in SEQ ID NO:86 wherein N is a restriction site;
A 5′ oligonucleotide primer having a sequence as shown in SEQ ID NO:87 wherein N is a restriction site, and a 3′ oligonucleotide primer having a sequence in SEQ ID NO:88 wherein N is a restriction site;
A 5′ oligonucleotide primer having a sequence as shown in SEQ ID NO:89 wherein N is a restriction site, and a 3′ oligonucleotide primer having a sequence in SEQ ID NO:90 wherein N is a restriction site;
A 5′ oligonucleotide primer having a sequence as shown in SEQ ID NO:91 wherein N is a restriction site, and a 3′ oligonucleotide primer having a sequence in SEQ ID NO:93 wherein N is a restriction site;
A 5′ oligonucleotide primer having a sequence as shown in SEQ ID NO:95 wherein N is a restriction site, and a 3′ oligonucleotide primer having a sequence in SEQ ID NO:94 wherein N is a restriction site;
A 5′ oligonucleotide primer having a sequence as shown in SEQ ID NO:97 wherein N is a restriction site, and a 3′ oligonucleotide primer having a sequence in SEQ ID NO:96 wherein N is a restriction site; or
A 5′ oligonucleotide primer having a sequence as shown in SEQ ID NO:99 wherein N is a restriction site, and a 3′ oligonucleotide primer having a sequence in SEQ ID NO:98 wherein N is a restriction site.

6. The isolated DNA molecule of claim 5, which can be amplified and/or cloned by the polymerase chain reaction from a Helicobacter pylori genome.

7. The isolated polynucleotide of claim 1, which is a DNA molecule that encodes the mature form or a derivative of a polypeptide encoded by the DNA molecule of claim 5.

8. The isolated polynucleotide of claim 1, which is a DNA molecule that encodes the mature form or a derivative of a polypeptide encoded by the DNA molecule of claim 6.

9. A compound, in a substantially purified form, that is the mature form or a derivative of a polypeptide comprising an amino acid sequence that is homologous to an amino acid sequence of a polypeptide associated with the Helicobacter membrane, which is selected from the group consisting of:
(a) the amino acid sequences as shown:
in SEQ ID NO:2, beginning with an amino acid in position −27 and ending with an amino acid in position 160 (HPO101);
in SEQ ID NO:4, beginning with an amino acid in position 1 and ending with an amino acid in position 172 (HPO104);
in SEQ ID NO:6, beginning with an amino acid in position −17 and ending with an amino acid in position 169 (HPO116);
in SEQ ID NO:8, beginning with an amino acid in position −21 and ending with an amino acid in position 198 (HPO121);
in SEQ ID NO:10, beginning with an amino acid in position −20, and ending with an amino acid in position 132 (HPO132);
in SEQ ID NO:12, beginning with an amino acid in position 1 and ending with an amino acid in position 114 (HPO15);
in SEQ ID NO:14, beginning with an amino acid in position −17 and ending with an amino acid in position 248 (HPO18);
in SEQ ID NO:16, beginning with an amino acid in position −40 and ending with an amino acid in position 74 (HPO38);
in SEQ ID NO:18, beginning with an amino acid in position −31 and ending with an amino acid in position 226 (HPO42);
in SEQ ID NO:20, beginning with an amino acid in position −21 and ending with an amino acid in position 179 (HPO45);
in SEQ ID NO:22, beginning with an amino acid in position −33 and ending with an amino acid in position 114 (HPO50);
in SEQ ID NO:24, beginning with an amino acid in position −60 and ending with an amino acid in position 349 (HPO54);
in SEQ ID NO:26, beginning with an amino acid in position −18 and ending with an amino acid in position 288 (HPO57);
in SEQ ID NO:28, beginning with an amino acid in position −21 and ending with an amino acid in position 150 (HPO58);
in SEQ ID NO:30, beginning with an amino acid in position −20 and ending with an amino acid in position 309 (HPO64);
in SEQ ID NO:32, beginning with an amino acid in position −35 and ending with an amino acid in position 129 (HPO70);
in SEQ ID NO:34, beginning with an amino acid in position −19 and ending with an amino acid in position 153 (HPO71);
in SEQ ID NO:36, beginning with an amino acid in position −25 and ending with an amino acid in position 176 (HPO76);
in SEQ ID NO:38, beginning with an amino acid in position −21 and ending with an amino acid in position 156 (HPO7);
in SEQ ID NO:40, beginning with an amino acid in position 1 and ending with an amino acid in position 144 (HPO80);
in SEQ ID NO:42, beginning with an amino acid in position −20 and ending with an amino acid in position 152 (HPO87);
in SEQ ID NO:44, beginning with an amino acid in position −31 and ending with an amino acid in position 112 (HPO95);
in SEQ ID NO:46, beginning with an amino acid in position −20 and ending with an amino acid in position 91 (HPO98);
in SEQ ID NO:48, beginning with an amino acid in position −21 and ending with an amino acid in position 129 (HPO9); and
(b) the amino acid sequences encoded by the Helicobacter DNA inserts found in American Type Culture Collection deposit numbers 98197 (HPO76), 98210 (HPO18), 98201 (HPO121), 98208 (HPO45), 98198 (HPO101), 98200 (HPO116), 98211 (HPO7), 98199 (HPO104), 98214 (HPO15), 98206 (HPO58), 98202 (HPO132), 98203 (HPO9), 98204 (HPO38), 98205 (HPO87), 98217 (HPO71), 98219 (HPO70), 98215 (HPO80), 98216 (HPO95), 98218 (HPO98), 98220 (HPO57), 98207 (HPO50), 98213 (HPO64), 98212 (HPO54), and 98209 (HPO42).

10. The compound of claim 9, which is the mature form or a derivative of a polypeptide encoded by a DNA molecule of claim 5.

11. The compound of claim 9, which is the mature form or a derivative of a polypeptide encoded by a DNA molecule of claim 6.

12. A method of preventing or treating Helicobacter infection in a mammal, said method comprising administering to said mammal a prophylactically or therapeutically effective amount of a compound of claim 9.

13. The method of claim 12, further comprising administering an antibiotic, an antisecretory agent, a bismuth salt, or a combination thereof.

14. The method of claim 13, wherein said antibiotic is selected from the group consisting of amoxicillin, clarithromycin, tetracycline, metronidizole, and erythromycin.

15. The method of claim 13, wherein said bismuth salt is selected from the group consisting of bismuth subcitrate and bismuth subsalicylate.

16. The method of claim 13, wherein said antisecretory agent is a proton pump inhibitor.

17. The method of claim 16, wherein said proton pump inhibitor is selected from the group consisting of omeprazole, lansoprazole, and pantoprazole.

18. The method of claim 13, wherein said antisecretory agent is an H₂-receptor antagonist.

19. The method of claim 18, wherein said H₂-receptor antagonist is selected from the group consisting of ranitidine, cimetidine, famotidine, nizatidine, and roxatidine.

20. The method of claim 13, wherein said antisecretory agent is a prostaglandin analog.

21. The method of claim 20, wherein said prostaglandin analog is misoprostil or enprostil.

22. The method of claim 12, which further comprises administering a prophylactically or therapeutically effective amount of a second Helicobacter polypeptide or a derivative thereof.

23. The method of claim 22, wherein the second Helicobacter polypeptide is a Helicobacter urease, a subunit, or a derivative thereof.

24. A composition comprising a compound of claim 9, together with a physiologically acceptable diluent or carrier.

25. The composition of claim 24, further comprising an adjuvant.

26. The composition of claim 24, further comprising a second Helicobacter polypeptide or a derivative thereof.

27. The composition of claim 26, wherein said second Helicobacter polypeptide is a Helicobacter urease, or a subunit or a derivative thereof.

28. A method of preventing or treating Helicobacter infection in a mammal, said method comprising administering to said mammal a prophylactically or therapeutically effective amount of a polynucleotide of claim 1.

29. A method of preventing or treating Helicobacter infection in a mammal, said method comprising administering to said mammal a prophylactically or therapeutically effective amount of a polynucleotide of claim 5.

30. A method of preventing or treating Helicobacter infection in a mammal, said method comprising administering to said mammal a prophylactically or therapeutically effective amount of a polynucleotide of claim 8.

31. A composition comprising a viral vector, in the genome of which is inserted a DNA molecule of claim 4, said DNA molecule being placed under conditions for expression in a mammalian cell and said viral vector being admixed with a physiologically acceptable diluent or carrier.

32. The composition of claim 31, wherein said viral vector is a pox virus.

33. A composition that comprises a bacterial vector comprising a DNA molecule of claim 4, said DNA molecule being placed under conditions for expression and said bacterial vector being admixed with a physiologically acceptable diluent or carrier.

34. The composition of claim 33, wherein said vector is selected from the group consisting of Shigella, Salmonella, Vibrio cholerae, Lactobacillus, Bacille bilié de Calmette-Guérin, and Streptococcus.

35. A composition comprising a polynucleotide of claim 1, together with a physiologically acceptable diluent or carrier.

36. The composition of claim 35, wherein said polynucleotide is a DNA molecule that is inserted in a plasmid that is unable to replicate and to substantially integrate in a mammalian genome and is placed under conditions for expression in a mammalian cell.

37. An expression cassette comprising a DNA molecule of claim 4, said DNA molecule being placed under conditions for expression in a procaryotic or eucaryotic cell.

38. A process for producing a compound of claim 9, which comprises culturing a procaryotic or eucaryotic cell transformed or transfected with an expression cassette of claim 37, and recovering said compound from the cell culture.

39. A method of preventing or treating Helicobacter infection in a mammal, said method comprising administering to said mammal a prophylactically or therapeutically effective amount of an antibody that binds to the compound of claim 9.


SEQUENCE LISTING (146 sequences)

SEQ ID NO: 1 - DNA, 982 bp, Helicobacter pylori
  CDS (320)...(880)
  sig_peptide (320)...(400)
  mat_peptide (401)...(880)
agattagcagcagcagggatttttaaattcctggccaacaggggcggttggaaaaaaatacgattaaaaaggcaaacgctttgaaagtattttttcatagaaattcccttttgttaaatgattgaagttggtgattatacctatttgtatcttaaaaatttgattttaaaagtttgagatggttttgtaggtgtatcccacttatccaatttatatcaatattttcactctaaaaccctcatccttgataaaaaattaaaccttttagaaaaataaccgattttagggtgtaactttaattcaacaagaaggatttattatgattaaaagaattgcttgtattttaagcttgagtgcgagtttagcgctggctggcgaagtgaatgggtttttcatgggtgcgggttatcagcaaggtcgttatggtccttataacagcaattactctgattggcgccatggcaatgatctttatggtttgaatttcaaattaggttttgtaggctttgccaataaatggtttggggctagggtgtatggctttttagattggtttaacacttcagggacagaacacaccaaaaccaatttgctcacctatggtggcggtggcgatttgattgtcaatctcattcctttggataaattcgctctaggtctcatcggtggcgttcaattagccggaaacacttggatgttcccttatgatgtcaatcaaacgagattccagttcttatggaatttaggcggaagaatgcgtgttggggatcgcagtgcgtttgaagcaggcgtgaaattccctatggttaatcaaggcaacaaagatgttagggcttatccgctactattcttgggtatgtggattatgttcttcactttctaatttattcctttcattcgctcttcttcatcaaatcaaccctaacccactcttaaaaggttggggttcaaaaatctttttcataaataaaatttgccttaa

SEQ ID NO: 2 - protein, 187 aa, Helicobacter pylori
MIKRIACILSLSASLALAGEVNGFFMGAGYQQGRYGPYNSNYSDWRHGNDLYGLNFKLGFVGFANKWFGARVYGFLDWFNTSGTEHTKTNLLTYGGGGDLIVNLIPLDKFALGLIGGVQLAGNTWMFPYDVNQTRFQFLWNLGGRMRVGDRSAFEAGVKFPMVNQGNKDVRAYPLLFLGMWIMFFTF

SEQ ID NO: 3 - DNA, 843 bp, Helicobacter pylori
  CDS (262)...(777)
ccaatggaggcgtttccaaaaacccaaacgggcgctttttaaagaaaaatctcaaaaaattcagggagcaagcggtaaaaatcgtagaaaaacgcttgataaaagagaatatgcaactgagcgattttaatgaagaagaattaaaaatcatgtttgaagctgaagaaaaaaggttgttagagcaaatccacgctaaagaattgaaagaaaagcaagaaaaaaccaccaagcattttaaagaagtttgggaaaagggcgaaaatgagcaagaaaaatagcgtaatttctggtttaatgaatttttttagcgaaaagaatgaacgctggctcttagcccacaggcacacgagagggtttgtgatagtggcgtggctttttcggtttaaaagcattgcgttttctattttgatcactctgttggttattttagtggatatttgggtttatagcgatgtgcgtcagtttttattggacacttctagctcttttatttggcttttgatcgctttactaatcaagtggggcgtgattgtcataagcgcacgtaaatgctaccaattcagccaaaaaatgtttacgctcattcaaagaaaaaggcaaatcagagagaatttaaaaaaccgctccaactacaaagataccaaaaatgcggaaaaactctctagcatcgctgaagaaatcatttcaaaaaaacaagaagagtcccgccccaaagaagattctaatcatgaaaaccataaagaaaagctttctaacattaccgaagaaagtgattcttaaaaaacaagaggaattgaaaagctaaaaaggatagggggggattacccaaagcatattggaggg

SEQ ID NO: 4 - protein, 172 aa, Helicobacter pylori
MSKKNSVISGLMNFFSEKNERWLLAHRHTRGFVIVAWLFRFKSIAFSILITLLVILVDIWVYSDVRQFLLDTSSSFIWLLIALLIKWGVIVISARKCYQFSQKMFTLIQRKRQIRENLKNRSNYKDTKNAEKLSSIAEEIISKKQEESRPKEDSNHENHKEKLSNITEESDS

SEQ ID NO: 5 - DNA, 904 bp, Helicobacter pylori
  CDS (248)...(805)
  sig_peptide (248)...(298)
  mat_peptide (299)...(805)
aaaaaggcccccatttttaaaagaaaatggggggctttaacaaggaagtgaacttgttgtatgaagaaaattctaaagaacgcgatcaaaaaagcgcatcaaagtttatccactatccctctaagttcttcactctatgctataatctctgttttaaaacattatggcgtgttagaagatattcagcaaaacccttccaaaccaaccaatctaaagaaagaaaccattcaaggaacgcattgatttgatgaataaaccatttttaatcttactcatagccctaattgtctttagcggctgtaacatgagaaaatatttcaaacccgctaaacaccaagttaaaggcgaagcgtatttccctaatcatttgcaagaaagtatcgtttcgtctaatcgttatggagccattttgaaaaatggagcggttataggcgataaaggtttaacgcagctaagaatcggtaagaatttcaattatgaaagcagttttttaaatgagagtcaggggtttttcatccttgcgcaagattgtttgaacaagattgataaaaaaacaagcaaaaacaaggtggctaaaagtgaggaaacggagctgaaattaaagggcgttgaagccgaagtccaagataaagtctgtcatcaagtggaattgattagcaataaccctaacgccagccaacaatctatcgttatccctttggagacttttgccttgagcgcaagcgttaaagggaatcttttagcggtggtgtttagcggacaattcagcgaatttatacgacatcacttctcaaaaattgctttttagtgagaaaggttccccaagcaccacgatcaattctttaatggcgaatgcctatttttaatggatacggtccttgtgtttcccccaagccaaaatggg

SEQ ID NO: 6 - protein, 186 aa, Helicobacter pylori
MNKPFLILLIALIVFSGCNMRKYFKPAKHQVKGEAYFPNHLQESIVSSNRYGAILKNGAVIGDKGLTQLRIGKNFNYESSFLNESQGFFILAQDCLNKIDKKTSKNKVAKSEETELKLKGVEAEVQDKVCHQVELISNNPNASQQSIVIPLETFALSASVKGNLLAVVFSGQFSEFIRHHFSKIAF

SEQ ID NO: 7 - DNA, 874 bp, Helicobacter pylori
  CDS (146)...(802)
  sig_peptide (146)...(208)
  mat_peptide (209)...(802)
gcaaaaatttggctggaaagcccctaggttggatccaattttgataaggatttttaaccggcaatttaaaaactattactccctgggatggttcataatgcaaaaaagccaacgcaacttaatacttcattaaggtttaatcacaatggataaaaacaacaacacgaatcttattttagcgatcgctctgtctttcttgtttatcgctctttataggtattttttccaaaaaccaaacaaaacaacaacccaaaccacaaagcaagaaacagccaacaaccacacagcaacaagtcctaacgcgcccaacgcccaaaattttagcgttactcaaaccatcccccaagagagtttgttaagcacgatttcttttgagcatgccaggattgaaattgattctttagggcgcatcaaacaggtttatctcaaggataaaaagtatctaacccctaaacaaaagggctttttagagcatgtgagccatctttttaaccccaaagctaacccgcaaccccccctaaaagagctcccccttttagcggccgataaactcaagcctttagaagtgcgttttttagaccccacgctcaataacaaagcgttcaacaccccttatagtgcttcaaaaaccactcttgggcctaatgaacagcttgttttaacccaagatttaggcgctcttaccatcattaaaaccctgactttttatgatgatttgcattatgatttaagaatcgccttcaaatcgcctaacaatattatccctagctatgtgatcactaatggttacagaccgggtggctgatttggacagctacaccttttcgggcgtgcatattagaaaacaacgacgaaaaattaagggtattgaaca

SEQ ID NO: 8 - protein, 219 aa, Helicobacter pylori
MDKNNNTNLILAIALSFLFIALYRYFFQKPNKTTTQTTKQETANNHTATSPNAPNAQNFSVTQTIPQESLLSTISFEHARIEIDSLGRIKQVYLKDKKYLTPKQKGFLEHVSHLFNPKANPQPPLKELPLLAADKLKPLEVRFLDPTLNNKAFNTPYSASKTTLGPNEQLVLTQDLGALTIIKTLTFYDDLHYDLRIAFKSPNNIIPSYVITNGYRPGG

SEQ ID NO: 9 - DNA, 761 bp, Helicobacter pylori
  CDS (303)...(758)
  sig_peptide (303)...(362)
  mat_peptide (363)...(758)
taaaaaatgcaaaaacatttttgggttattaaaaagattccttctaggagtcgaacctgccaggcatgcaaggtagctttcgcgagctttgagatcccttcaaacgctttgattagaaacgggaaagattacctggtgtttgtgagaacgcctaaaggttttaggcctgtggtggttcaagttttagaagagcgcagcaagatttttatcgtgaacgctcaaaatttacaccctaatgacagcgtggcagtggggtcattgatagggttaaaaggcatgatcaacaatttaggggaagaataatgctcgcttccattattgaattttccttacgccagcgaataatcgtgattgttggcgcgattcttattttgttttttgggacttatagttttatccacactccagtagatgctttcccggatatttcgcccactcaagtcaaaatcattttaaaactccccggttctagccctgaagaaatggaaaataacatcgtgcgccctttagaattggagcttttaggcttgaaagggcaaaaatctttaagaagtatttcaaaatattctatttcagacatcacgatagattttgatgacagcgtggatatttatttagcgagaaacattgttaatgagcgcttgagcagcgtgatgaaagatttacccgtgggggttgaaaggggcatggcgcccattgttacgccgctatcaaatatctttatgtttcactattggatgggccatatccctggagaatag

SEQ ID NO: 10 - protein, 152 aa, Helicobacter pylori
MLASIIEFSLRQRIIVIVGAILILFFGTYSFIHTPVDAFPDISPTQVKIILKLPGSSPEEMENNIVRPLELELLGLKGQKSLRSISKYSISDITIDFDDSVDIYLARNIVNERLSSVMKDLPVGVERGMAPIVTPLSNIFMFHYWMGHIPGE

SEQ ID NO: 11 - DNA, 392 bp, Helicobacter pylori
  CDS (25)...(366)
gaattgaaccatgctgaagggtgtatggtattaggaagcttataccatcatggcttaggcacgcctaaggattcaagaaaggctcttgatttgtatgaaaaagcttgcgatttaaaagacagccccgggtgcattaatgcaggatacatgtatggtgtagcaaagaattttaaggaggctattgttcgttattctaaggcatgcgaattgaaagatggcagggggtgttataatttaggggttatgcaatacaacgcccaaggcacagcaaaggacgaaaagcaagcggtagaaaactttaaaaaaggttgcaaatcaagcgttaaagaagcatgcgacgctctcaaggaattaaaaatagaactttagttttaacgaagttaagctaaagg

SEQ ID NO: 12 - protein, 114 aa, Helicobacter pylori
MVLGSLYHHGLGTPKDSRKALDLYEKACDLKDSPGCINAGYMYGVAKNFKEAIVRYSKACELKDGRGCYNLGVMQYNAQGTAKDEKQAVENFKKGCKSSVKEACDALKELKIEL

SEQ ID NO: 13 - DNA, 982 bp, Helicobacter pylori
  CDS (117)...(911)
  sig_peptide (117)...(167)
  mat_peptide (168)...(911)
ccacatttaaggtagaaaccactcaattagatgtaaaaattccaaacggcaaccaaaaaatggttaaaaaggacacaataaaccccaaaaatgaaatttaaatatatgggaacttaatgagaattttttttgttatcatgggacttgtgttttttggttgcaccagtaaggtgcatgagatgaaaaaaagcccttgcaccttgtatgaaaacaggttaaatctcgcagaaatctttcacaagcgagcaattgatctatttagagagcttttaagccaccaagaaaagcatttagaaaacaagctttctggtttttcggtgagtgatttggacatgcaaagcgtgtttcggctggaaagaaaccgcttgaaaatcgcttacaagctcttaggcttgatgagttttatcgctcttattttagcgatcgtgttaatcagtcttctacccttacaaaaaaccgaacaccatttcgtggattttttaaaccaggacaagcattacgtcattatccaaagagcggataaaagcatttccagtaatgaagcgttggctcgttcgctcattggggcgtatgtgttaaaccgagagagcattaaccgcattgacgataaatcgcgctatgaattggtgcgcttgcaaagcagttctaaagtgtggcaacgctttgaagatttgattaaaacccaaaacagcatttatgtgcaaagccatttggaaagagaagtccatatcgtcaatattgcgatctatcagcaagacaataaccccattgcgagcgtctccattgccgctaaacttttgaatgaaaacaagctggtgtatgaaaagcgttataaaatcgtattgagttatttgtttgacaccccgatgaattcaagcttgcaagcttgcaagctctcaggcttcatagtttgacatgacatatagatgagctttatgcggtacgattatcacagaatggctaacgcagcaggcaccgagta

SEQ ID NO: 14 - protein, 265 aa, Helicobacter pylori
MRIFFVIMGLVFFGCTSKVHEMKKSPCTLYENRLNLAEIFHKRAIDLFRELLSHQEKHLENKLSGFSVSDLDMQSVFRLERNRLKIAYKLLGLMSFIALILAIVLISLLPLQKTEHHFVDFLNQDKHYVIIQRADKSISSNEALARSLIGAYVLNRESINRIDDKSRYELVRLQSSSKVWQRFEDLIKTQNSIYVQSHLEREVHIVNIAIYQQDNNPIASVSIAAKLLNENKLVYEKRYKIVLSYLFDTPMNSSLQACKLSGFIV

SEQ ID NO: 15 - DNA, 760 bp, Helicobacter pylori
  CDS (236)...(577)
  sig_peptide (236)...(355)
  mat_peptide (356)...(577)
ctaaaaatggaagcctaaaaaggggtaaaaaagctttaaaaagggggcaaaaaattgaagcgatttcaaaaaaaaaaaaaaaaaacaatttcagtttcttattagctagatttgattagaataaaaagcttttatgtgtttaaacttcattgtcttaaaacttttaagagcaattttaaaattcgttggcgtataatatcaaatttgaatgaactgactaaaagggttttaaataatggctgaaaattctttcaaaaatgtttccacacaacccaaaccatttttcttattaccagttaaaaccctgtttcttttaggaggcgtttttagcgcgttttttatccttgttgctggcttggttttttttaattacactaattcaatggaccatgcgatttttaacttgatgcgttcaaactcttctaaccctattttagatcaaacgctccgacgcgttgtttttttaggctcttctcaattcgtgttgcctttgagcttgttagtgggggtttttttaagcttgtatcgtaaaaatttagcacttggggtgtggtttgtgctaaagcgtgatcttatttgaagcccttttagaatctttaaaacaccttttggcacactccattcaatgggttttcggcacaggctaatttccctagcactatcgggctttctttgacgggaattttatggggtgcttggttttaattaataccccatttgttccacggatcaaaaatttcaaaaacatttttttcgtaaa

SEQ ID NO: 16 - protein, 114 aa, Helicobacter pylori
MAENSFKNVSTQPKPFFLLPVKTLFLLGGVFSAFFILVAGLVFFNYTNSMDHAIFNLMRSNSSNPILDQTLRRVVFLGSSQFVLPLSLLVGVFLSLYRKNLALGVWFVLKRDLI

SEQ ID NO: 17 - DNA, 939 bp, Helicobacter pylori
  CDS (37)...(816)
  sig_peptide (37)...(138)
  mat_peptide (139)...(816)
agtttgcaaccctggatgagagtagtggcagagtttatgctgattccgttaaaaacattcctaaaaatattattgaaaatattcctaaaaactttccaaaagatttggatagtttgcgttgttatttgggggttgggttgtagttttttaaacgctaacagcattcaattagaagaaacgctcagacgaaatcctaaaaatcttatttggcaacactttaaaaagaagtttaaaaagagcaacacgatcccttatgccccaaacagccgttggaaatatttaggcacgagcatagggattttaggcgtgtcgttggtgatagggattgtggggttgtatctcatgccagagagcgtaacgaattgggatagagaaaagtttggcgtcaaaagttggtttgaaaatgtccgcatggggccaaaactggacaatgatagttttattttcaatgaaattttgcacccttattttggggctatgtattatatgcaaccgcgcatggctgggtttggctggatggcatcagcgtttttttcttttatcacttctacgcttttttgggaatatggcttggaagcgtttgtggaagtgcctagctggcaggatttagtgatcacgcctttattaggctccattttaggggaagggttttatcagctcacgcgctatatccaacggaaccaaggcaagctttttggctctttatttttagggcgtttagccatcgctcttatggatcctatcggttttgtgatcagggatttagggcttggggaagctttaggattcataataaacatgaaatccgttccagtttaagccctaatggcttgaatttgacttacaaattctaaagaataacccgcatcaagcgactttaaaaacattttcaccaatgaaataccccgctctatctatgaaccgactatacaaacgaac

SEQ ID NO: 18 - protein, 260 aa, Helicobacter pylori
MLIPLKTFLKILLKIFLKTFQKIWIVCVVIWGLGCSFLNANSIQLEETLRRNPKNLIWQHFKKKFKKSNTIPYAPNSRWKYLGTSIGILGVSLVIGIVGLYLMPESVTNWDREKFGVKSWFENVRMGPKLDNDSFIFNEILHPYFGAMYYMQPRMAGFGWMASAFFSFITSTLFWEYGLEAFVEVPSWQDLVITPLLGSILGEGFYQLTRYIQRNQGKLFGSLFLGRLAIALMDPIGFVIRDLGLGEALGFIINMKSVPV

SEQ ID NO: 19 - DNA, 603 bp, Helicobacter pylori
  CDS (1)...(600)
  sig_peptide (1)...(63)
  mat_peptide (64)...(600)
atgaaaagatttgatttgtttttatcactcatgggtgtttgcgtttgcgttcaaacttacgccgagcaagattacttttttagggattttaaatctaaagacttgccccaaaaactccatcttgataaaaagctctcccaaacaatacagccatgcgcgcaacttaacgcatcaaaacactacactgctaccggggttagagagcctgataaatgcacaaagagttttaaaaaatccgctatcatgtcctatgacttagcgctaggctattgggtgagccaaaacaaacaatacggctttaaagctatagaaaatttaaacgcttgggctaaagagcttcaaagcgtggatacttatcagagcgaggataatatcaatttctacatgccttatatgaacatggcttattggtttgtcaaaaaggcattccctagcccagaatacgaagatttcgttaagcggatgcgccaatattctcaatcagctcttaacactaaccatggggcgtggggcattctctttgatgtgagctctgcactagcgttagatgatcatgctcttttgcacaatagcgctaatcggtggcaggattgggatatttaa

SEQ ID NO: 20 - protein, 200 aa, Helicobacter pylori
MKRFDLFLSLMGVCVCVQTYAEQDYFFRDFKSKDLPQKLHLDKKLSQTIQPCAQLNASKHYTATGVREPDKCTKSFKKSAIMSYDLALGYWVSQNKQYGFKAIENLNAWAKELQSVDTYQSEDNINFYMPYMNMAYWFVKKAFPSPEYEDFVKRMRQYSQSALNTNHGAWGILFDVSSALALDDHALLHNSANRWQDWDI

SEQ ID NO: 21 - DNA, 704 bp, Helicobacter pylori
  CDS (157)...(597)
  sig_peptide (157)...(255)
  mat_peptide (256)...(597)
aaaatcgctcataaaatcgtgttttttgataccggtaagatcgctgaagaaaacagcgctaaagaattttttaaccacccgaaatctcaaagagtgcaaaaatttttagaaactttccattttttagggagctgttaaataaagtttgctaaaaagatggttttaatttcaaaaagaggtgtttttatgaaaacaaacgggctttttaaaatgtgggggctgtttttagttttaatcgctttactctttaacgcatgctctgatagccataaagaaaaaaaggacgctttagaagtcattaaacaaagaggggttttaaaagtgggggtttttagcgataagcctccttttggatctgtggattctaaagggaaatatcaaggctatgatgtgatcatcgctaaacgcatggcccttgatttattgggcgatgaaaataagattgagtttatacccgtagaagcttcagctagggtggaatttttaaaagccaataaagtggatattatcatggctaatttcacgcgcactaaagaaagagaaaaagtcgtggatttcgctaatccgtatatgaaagtcgctttgggggttgatttctaaagatggggtcattaaaaatatagaagagttgaaggataaagagttgattgtgaataaaggcacgacagcggatttttatttcactaaaaattaccccaatatca

SEQ ID NO: 22 - protein, 147 aa, Helicobacter pylori
MVLISKRGVFMKTNGLFKMWGLFLVLIALLFNACSDSHKEKKDALEVIKQRGVLKVGVFSDKPPFGSVDSKGKYQGYDVIIAKRMALDLLGDENKIEFIPVEASARVEFLKANKVDIIMANFTRTKEREKVVDFANPYMKVALGVDF

SEQ ID NO: 23 - DNA, 1306 bp, Helicobacter pylori
  CDS (40)...(1266)
  sig_peptide (40)...(219)
  mat_peptide (220)...(1266)
tttgacagcttatcatttggcaataaaacaccaaaatgaatgagttacacaaaaaaatactcaacaccacccaaccggcgtaaaatgcaaaacattatcgctattaaaagatcctctagagtcgacctgcaggcatgcaagctagctttcgcgagctcgagatcacccatgcaatttcaaaaaaccttatttcctttacccttattatttttatcttgttgtatcgctgaagaaaatggggcgtatgcgagcgtggggtttgaatattccattagtcatgccgttgagcataataacccttttttaaatcaagaacgcatccaaatcatttctaacgctcaaaacaaaatctataaactcaatcaagtcaaaaatgaaatcacaagcatgcaaaacacctttaattacatcaacaacgctttaaaaaacaatgctaaattaacccccactgaaatccaagctgagaaatactacctccaatccacccttcaaaacattgaaaaaatagtcacacttagcggtggcgttgcatctaaccccaaactagtccaagcgttggaaaaaatgcaagaacccattactaaccctttagaattagcagaaaacttaagaaatttagaattgcaatttgctcaatctcaaaaccgcatgctttcttctttatcttctcaaaccgctcaaatttcaaattctttgaacgcgcttgatcccagctcttattctaaaaacatttcaagcatgtctggggtgagtttgagcgtagggtataagcatttctttactaagaaaaaaaatcaagggtttcgctattacttgttttatgactatggttacactaactttggttttgtgggtaatggctttgatggtttaggcaaaatgaataaccacctctatgggcttggaataaactacctttataatttcattgataatgcacaaaaacattcgagcgtgggtttttatgcgggttttgctttggcggggaattcgtgggtagggaatggtttaggcatgtgggtgagccaaacggattttatcaacaattacttgatgggctatcaagctaaaatacacacgaactttttccagatccctttgaattttggggttcgtgtgaatgtcaataggcataacggatttgaaatgggcctaaaaatccctttagcggtgaattccttttatgaaacgcatggcaaagggttaaacacttccctctttttcaaacgccttgtggtgtttaatgtgagttatgtttatagtttttagggggtaaatgccttcaaacgctcttttgattgaagaa

SEQ ID NO: 24 - protein, 409 aa, Helicobacter pylori
MSYTKKYSTPPNRRKMQNIIAIKRSSRVDLQACKLAFASSRSPMQFQKTLFPLPLLFLSCCIAEENGAYASVGFEYSISHAVEHNNPFLNQERIQIISNAQNKIYKLNQVKNEITSMQNTFNYINNALKNNAKLTPTEIQAEKYYLQSTLQNIEKIVTLSGGVASNPKLVQALEKMQEPITNPLELAENLRNLELQFAQSQNRMLSSLSSQTAQISNSLNALDPSSYSKNISSMSGVSLSVGYKHFFTKKKNQGFRYYLFYDYGYTNFGFVGNGFDGLGKMNNHLYGLGINYLYNFIDNAQKHSSVGFYAGFALAGNSWVGNGLGMWVSQTDFINNYLMGYQAKIHTNFFQIPLNFGVRVNVNRHNGFEMGLKIPLAVNSFYETHGKGLNTSLFFKRLVVFNVSYVYSF

SEQ ID NO: 25 - DNA, 999 bp, Helicobacter pylori
  CDS (25)...(942)
  sig_peptide (25)...(78)
  mat_peptide (79)...(942)
tgacttagttttgtaaggaatgagatgataaagagttggactaaaaagtggtttttgattttatttttaatggcaagctgtttcagttatttggtggctacaaccggtgagaaatattttaaaatggctactcaagcctttaagagaggggattaccataaagcggtggctttttataagaggagctgtaatttaagggtgggggttggttgcacgagtttgggctctatgtatgaagatggcgatggcgtggatcagaatattccaaaagccgttttttattacagaagagggtgcaatttgaggaattatttggcttgtgcgagtttaggctctatgtatgaagatggcgatggcgttcaaaaaaaccttccaaaggctatctattattataggagagggtgccacttaaagggtggggtgagctgcgggagtttaggttttatgtattttaatggcacgggcgttaagcaaaattatgccaaagccctttctctttctcaatacgcttgcagtttgaattatggcattagttgtaactttgtagggtatatgtataggagcgccaaaggcgtacagaaggatttgaaaaaagcccttacgaattttaaaagagggtgccatttaaaagacggagcgagctgtgtgagcttgggatacatgtatgaagtcggcatgtatgtcagacaaaatgaagagcaagccttgaatctttataaaaagggttgtcatttaaaagaagggagcggttgccataatgtggcggtgatgtattacacgggtaagggtgcttcaaaggatttagataaagccatttcgtattataagaaaggttgcactctaggctttagtggtagctgtaaggtgttagaagaagtgattggcaagaagtctgatgatttgcaagatgatgcgcaaaacgacacgcaagatgatacgcaataagccagagtttaggggctaatgattaaaactcatcttatagaaatctttctattc

SEQ ID NO: 26 - protein, 306 aa, Helicobacter pylori
MIKSWTKKWFLILFLMASCFSYLVATTGEKYFKMATQAFKRGDYHKAVAFYKRSCNLRVGVGCTSLGSMYEDGDGVDQNIPKAVFYYRRGCNLRNYLACASLGSMYEDGDGVQKNLPKAIYYYRRGCHLKGGVSCGSLGFMYFNGTGVKQNYAKALSLSQYACSLNYGISCNFVGYMYRSAKGVQKDLKKALTNFKRGCHLKDGASCVSLGYMYEVGMYVRQNEEQALNLYKKGCHLKEGSGCHNVAVMYYTGKGASKDLDKAISYYKKGCTLGFSGSCKVLEEVIGKKSDDLQDDAQNDTQDDTQ

SEQ ID NO: 27 - DNA, 805 bp, Helicobacter pylori
  CDS (130)...(642)
  sig_peptide (130)...(192)
  mat_peptide (193)...(642)
tagtgagagtcgcccaattccaatgatggaaagcatgcaagtctttcatgatatatcgccatttttaaaaagttttgagactataacccttaaaaaactatcgcaacaaatcaattcataggaacaagcatgttttttaaaacttatcaaaaattactgggtgcaagctgtttggcgttatatttagcaggctgtgggggcgacagtggcgaaccactagttgggattgaaaaaaatagcttcaattctaccttgaaaatcatttctaaaaccgacaacatagaaatccaagacttgaagctcaatcgtgagaattgcgagcatgatgaaaatttcttggtaaagttaatacaagaaacagccaatacatacctgtttgcatcagaaaaagaaaaagcgattaaaaaccaccaagcaaaaatcgcaagacttcaaaaaagaaggagcggttgccataatgtggcggtgatgtattacacgggtaagggtgcttcaaaggatttagataaagccatttcgtattataagaaaggttgcactctaggctttagtggtagctgtaaggtgttagaagaagtgattggcaagaagtctgatgatttgcaagatgatgcgcaaaacgacacgcaagatgatacgcaataagccagagtttaggggctaatgattaaaactcatcttatagaaatctttctattctcttgctatcaaatagggattgagcgtctctattgatgggtattgagattaaaaacctgcaaatctagggtttggatttattctcatatggataataaaaatactc

SEQ ID NO: 28 - protein, 171 aa, Helicobacter pylori
MFFKTYQKLLGASCLALYLAGCGGDSGEPLVGIEKNSFNSTLKIISKTDNIEIQDLKLNRENCEHDENFLVKLIQETANTYLFASEKEKAIKNHQAKIARLQKRRSGCHNVAVMYYTGKGASKDLDKAISYYKKGCTLGFSGSCKVLEEVIGKKSDDLQDDAQNDTQDDTQ

SEQ ID NO: 29 - DNA, 1101 bp, Helicobacter pylori
  CDS (40)...(1026)
  sig_peptide (40)...(99)
  mat_peptide (100)...(1026)
ggttataccgaaaaaacaatatgaaatcaaggagtttgtatgcaacagcgtcatttaggccctttaaaagtgggtgcattagctctagggtgcatgggcatgacttatgggtatggggaagtccatgataaaaagcagatggttaaacttatccataaggctttggaattgggtattaacttttttgacactgcagaggcttatggggaagataatgaaaagcttttagcgaagcgatcaagccttattaaagacaaggttgtggtagcgagcaagtttgggatttactacgcagatcctaatgacaaatacgcaaccatgtttttagactccagttctaaccgcattaagagtgccattgaagggagtttgaaacgcttaaaagtagaatgcattgatttatactaccaacaccgcatggatactaacacgcccatagaagaagtggcagaagttatgcaagctcttattaaagaaggaaaaattaaagcttgggggatgagtgaggcagggttatctagcatccaaaaagcccatcaaatttgccctttaagcgcgttgcagagcgaatattccttgtggtggcgcgaacctgaaaaagagattttaggttttttagaaaaagaaaaaattggctttgtcgctttttcgcctttgggtaaggggtttttaggcgcgaaatttgaaaaaaatgctaccttcgctagtgaagattttagaagcgtttctcctaggtttaatcaagaaaatctagccaaaaattacgtcttggtggaattaatccaagatcatgcacacgctaaaggcgttacaccagcccaactggctctctcgtggattttgcacacgcaaaaaatcattgtccctctctttggcaccaccaaagaatccaggctcatagaaaatataggggctttgcaggtttcttggagtcaaaaagaattggagatttttcaaaaagaattgactgcaatcaaaatagaaggggcccgctaccctgaaagaatcaatgaaatggtgaatcaataaaagtattgggtatttataattgcattggctcttttaaaagagattgagcgttatttcctgtttgtcagtgtg

SEQ ID NO: 30 - protein, 329 aa, Helicobacter pylori
MQQRHLGPLKVGALALGCMGMTYGYGEVHDKKQMVKLIHKALELGINFFDTAEAYGEDNEKLLAKRSSLIKDKVVVASKFGIYYADPNDKYATMFLDSSSNRIKSAIEGSLKRLKVECIDLYYQHRMDTNTPIEEVAEVMQALIKEGKIKAWGMSEAGLSSIQKAHQICPLSALQSEYSLWWREPEKEILGFLEKEKIGFVAFSPLGKGFLGAKFEKNATFASEDFRSVSPRFNQENLAKNYVLVELIQDHAHAKGVTPAQLALSWILHTQKIIVPLFGTTKESRLIENIGALQVSWSQKELEIFQKELTAIKIEGARYPERINEMVNQ

SEQ ID NO: 31 - DNA, 495 bp, Helicobacter pylori
  CDS (1)...(492)
  sig_peptide (1)...(105)
  mat_peptide (106)...(492)
atggtatttgacagaacaatcagcgtaagagaaaaaaaagcggctaaaacgcttgggattgtggggatcgtcttttttattttgtttggcatcgtaataagcggggtggcttttcaaaaagagtgggtgcaacaattggatttattttttatagacttgatccacaaccctgcccccattcaagggagcgcgtggctttctttcgtgttttttagcacatggtttgcgcaaagcaagctcaccactcctatagccttactcattggcttgtggtttgggtttcaaaaacgcatcgctttaggggtgtggtttttctttagcatcttattaggtgaattcaccttaaaatcccttaagcttttagtggcgcgcccacggcctgtaaccaatggcgaattggtttttgcacatggctttagtttccccagcgggcatgctttagcttccagcgcttttttacggctctttggcgtttgtttgttatgctattcctaa

SEQ ID NO: 32 - protein, 164 aa, Helicobacter pylori
MVFDRTISVREKKAAKTLGIVGIVFFILFGIVISGVAFQKEWVQQLDLFFIDLIHNPAPIQGSAWLSFVFFSTWFAQSKLTTPIALLIGLWFGFQKRIALGVWFFFSILLGEFTLKSLKLLVARPRPVTNGELVFAHGFSFPSGHALASSAFLRLFGVCLLCYS

SEQ ID NO: 33 - DNA, 569 bp, Helicobacter pylori
  CDS (1)...(516)
  sig_peptide (1)...(57)
  mat_peptide (58)...(516)
atgttgaaatttaaatatggtttgatttatatcgcgctcattataggacttcaagcgacagattatgacaatttagaagaagaaaaccaacaattagacgaaaaaataaaccatttaaagcaacagcttaccgaaaaaggggtttcgcccaaagagatggataaggataagtttgaagaagaatatttagagcgaacttacccaaagatttcttcaaagaaaagaaaaaaattactcaaatctttctccatagccgatgataagagtggggtttttttagggggtgggtatgcttatgggggatttaatctttcttatcaaggggagatgttagacaaatatggtgcgaatgcccctagtgtgtttaaaaacaatattaagattaacgctcctgtttctatgattagcgttaaattcgggtatcaaaaatactttgtgccttattttgggacacgattttatggggatttattgcttgggggtggagcgttaaaaaggatgcaagcaagcaacctgtaggctcgtttatttatgtttttaggggctatgaatacggatttattgtttga

SEQ ID NO: 34 - protein, 172 aa, Helicobacter pylori
MLKFKYGLIYIALIIGLQATDYDNLEEENQQLDEKINHLKQQLTEKGVSPKEMDKDKFEEEYLERTYPKISSKKRKKLLKSFSIADDKSGVFLGGGYAYGGFNLSYQGEMLDKYGANAPSVFKNNIKINAPVSMISVKFGYQKYFVPYFGTRFYGDLLLGGGALKRMQASNL

SEQ ID NO: 35 - DNA, 1416 bp, Helicobacter pylori
  CDS (315)...(917)
  sig_peptide (315)...(389)
  mat_peptide (390)...(917)
gtattaccgcctttgagtgagctgatacgaattagatcattaaaggttccttttggagcctttttttttgaattctcatgtttgacagcttatcatttggcaataaaacaccaaaatgaatgagttacacaaaaaaatactcaaacaccacccaaccggcgtaaaatgcaaaacattatcgctattaaaagatcctctagagtcgacctgcaggcatgcaagctagctttcgcgagctcgagatcattttagccataaaaagcttatgttttcattaaaaatgttatgatacgctcaaatagtcaagcaaaaaaatgtcaattaaaagggttagattgaaaatattcgttctgttgatgtcggtaattttaggaatatcattaacaggttgcataggctatcgtatggacttagaacattttaacacgctctattatgaagaaagccctaaacaagcttatgaatattctaaacaattcactaagaaaaaaaagaacgctcttttatgggacttgcaaaacggcttgagcgctttatacgccagagattaccagacttctttaggggtgttagatcaagccgagcaacgctttgataaaacccaaagcgctttcacaagaggggctggttatgtgggcgctaccatgattaatgataacgtgcgcgcttatggggggaatatttatgagggcgttttaatcaattattacaaagcgatagactacatgcttttaaacgatagcgcgaaagctagggtgcaattcaaccgcgcgaacgaacgccagcgcagggctaaagaattttattatgaggaagtgcaaaaagccattaaagagatcgattctagcaaaaagcacaatattaatatggaacgctctagggctagaagtgagcgagattttaaacaacacttattctaatttagacaaatacgaagcttatcaaggcttgcttaacccagcggtttcgtatctttcagggttgttttacgctttaaatggggataagaataaggggttaggctatcttaatgaagcctacgggatcagtcaaagcccttttgtagcccaagacttggttttttttaaaaaccctaataggagtcatttcacttggatcatcattgaagatggtaaagagccgcaaaaaagccaatttaaaattgatgtgcctatttttatgattgattcggtttataacgtgagtatagccttgcccaagctagaaaaaggggaagcgttttatcaaaatttcactcttaaagatggagaaaaagtaacgccctttgacactttagcctcaatagatgcggtggtcgctagcgaatttaggaagcagttaccctatattatcactagagccattctatcggctacttttagaggtgggcatgcaagcggtagcgaattattattt

SEQ ID NO: 36 - protein, 201 aa, Helicobacter pylori
MSIKRVRLKIFVLLMSVILGISLTGCIGYRMDLEHFNTLYYEESPKQAYEYSKQFTKKKKNALLWDLQNGLSALYARDYQTSLGVLDQAEQRFDKTQSAFTRGAGYVGATMINDNVRAYGGNIYEGVLINYYKAIDYMLLNDSAKARVQFNRANERQRRAKEFYYEEVQKAIKEIDSSKKHNINMERSRARSERDFKQHLF

SEQ ID NO: 37 - DNA, 738 bp, Helicobacter pylori
  CDS (201)...(731)
  sig_peptide (201)...(263)
  mat_peptide (264)...(731)
cgcctgattgttcctttccctgctatggaaaaagcgaaataaaaacaagaaagtaacctatgatttcgctcgtttgatggatggggctaaagaagtcaaatgctctgaattcgctagcgtgatgattgaaaacatgtgaaagagcgttttttaggctctggtatttgaatgcgattattaggctaatactatcataaggaatgaagttgataaaatttgtgcgtaatgtggttttattcattttaacagcgatctttttagcactcatgcttttagtgagctattgcatgccccattatagcgtggctgtcattagcggggtggaagtccaaagaatgaatgaaaatgcacgcccaaataataaggaagtaaaaaccctagctagagatgtctattttgtgcaaacttacgaccctaaggatcaaaaaagcgtaaccgtctatcgtaacgaagacacgcgctttggcttccctttttattttaagtttaattcggctgatatttcagccctcgctcaaagtttagtcaaccagcaagtggaagtgcaatactatggctggcggatcaatttgtttaacatgttccctaatgtgatttttttaaagcccttaaaagagagtgctgagatgtcaaaacccatttttagctggattttatacgcctggctactagtggggcttttttatcaagcgcgcgtcctgtttggaattttatttaaggggaaagctcaataaatcc

SEQ ID NO: 38 - protein, 177 aa, Helicobacter pylori
MKLIKFVRNVVLFILTAIFLALMLLVSYCMPHYSVAVISGVEVQRMNENARPNNKEVKTLARDVYFVQTYDPKDQKSVTVYRNEDTRFGFPFYFKFNSADISALAQSLVNQQVEVQYYGWRINLFNMFPNVIFLKPLKESAEMSKPIFSWILYAWLLVGLFYQARVLFGILFKGKAQ

SEQ ID NO: 39 - DNA, 435 bp, Helicobacter pylori
  CDS (1)...(432)
atgttagaaaaattgattgaaagagtgttgtttgccactcgttggttgctagcccctttatgtattgccatgtcgttagtgctggtggttttaggctatgtgttcatgaaagagttgtggcacatgctcagccatttaaacacgatcagcgaaacggatttggttttatcagccttaggattagtggatttgttgtttatggccgggcttgttttaatggtgttactcgccagttatgaaagctttgtttctaaattagacaaggtggatgccagtgaaatcacttggctaaagcacacggattttaacgctttaaaattaaaggtttcactctccattgtagcgatttcagcgattttcttgctcaaacgctacatgagtttagaaagatgttttatcccagcattccctaaggatacgccccctatcgcataa

SEQ ID NO: 40 - protein, 144 aa, Helicobacter pylori
MLEKLIERVLFATRWLLAPLCIAMSLVLVVLGYVFMKELWHMLSHLNTISETDLVLSALGLVDLLFMAGLVLMVLLASYESFVSKLDKVDASEITWLKHTDFNALKLKVSLSIVAISAIFLLKRYMSLERCFIPAFPKDTPPIA

SEQ ID NO: 41 - DNA, 519 bp, Helicobacter pylori
  CDS (1)...(516)
  sig_peptide (1)...(60)
  mat_peptide (61)...(516)
atgcgtttattattgtggtgggtattggtattatcgctctttttaaatcctttgagagcggttgaagagcatgaaacagatgcggtggatttgtttttgattttcaatcaaatcaaccaactcaatcaagtcattgaaacttataagaaaaaccctgaaagaagtgctgaaatctctctgtataacacccaaaagaatgatttgattaaaagtttgacttctaaagtgttgaatgaaagggataaaattggcattgatatcaatcaaaatttaaaagagcaagagaaaatcaaaaagcgcttgtctagaagcattaagggcgataatttctacactttcatgaaagacagattgtctttagatattttgttgatagatgaaattttgtatcgttttatagataaaatcaagagcagtattgatatttttagcgaacaaaaagatgtggaaagtttcagcgatgccttccttttgcgttttaggacaattccaactcataccctttccctaaaaatttaa

SEQ ID NO: 42 - protein, 172 aa, Helicobacter pylori
MRLLLWWVLVLSLFLNPLRAVEEHETDAVDLFLIFNQINQLNQVIETYKKNPERSAEISLYNTQKNDLIKSLTSKVLNERDKIGIDINQNLKEQEKIKKRLSRSIKGDNFYTFMKDRLSLDILLIDEILYRFIDKIKSSIDIFSEQKDVESFSDAFLLRFRTIPTHTLSLKI

SEQ ID NO: 43 - DNA, 432 bp, Helicobacter pylori
  CDS (1)...(429)
  sig_peptide (1)...(93)
  mat_peptide (94)...(429)
atgaaaaaatttttttctcaatctttattagctttgattgtgtctatgaacgcgctactggccatggatggcaatggcgtttttttaggggcgggttatttgcaagggcaagcccaaatgcatgcggatattaattctcaaaaacaagccactaacgctactatcaaaggctttgatgcgcttttagggtatcaatttttctttgggaaatactttggcttgcgtgcttatgggttttttgactacgctcatgccaattctattaggcttaaaaaccctaactataacagcgaagtggcgcaattggcgggtcaaattcttgggaaacaagaaatcaatcgcttaacgagccttgctgatcctaaaacctttgagccaaacatgctcacttatgggggggctatggatttaatggttaatgttcatcaataa

SEQ ID NO: 44 - protein, 143 aa, Helicobacter pylori
MKKFFSQSLLALIVSMNALLAMDGNGVFLGAGYLQGQAQMHADINSQKQATNATIKGFDALLGYQFFFGKYFGLRAYGFFDYAHANSIRLKNPNYNSEVAQLAGQILGKQEINRLTSLADPKTFEPNMLTYGGAMDLMVNVHQ

SEQ ID NO: 45 - DNA, 336 bp, Helicobacter pylori
  CDS (1)...(333)
  sig_peptide (1)...(60)
  mat_peptide (61)...(333)
atgaaaacctttaaaaacctgctctgttttagcctgatcgctatgagttggctccaagcggacatgttggataatttcactagggccattaacagctacaccactaaaaagcttaatgaaatcaaggatcaagtcaatagcgctaaccctactaaaaatcacaataccacttataacgctaatggcatgctcattaacattgattgtaaagtcttaaaaaataacttctattcggtgtgttattctagcgagttaaaaaaccctatttatggcgtgagcgtgttgtttggggatttagtggataaaaataatattgaaaaacgctatgagttttaa

SEQ ID NO: 46 - protein, 111 aa, Helicobacter pylori
MKTFKNLLCFSLIAMSWLQADMLDNFTRAINSYTTKKLNEIKDQVNSANPTKNHNTTYNANGMLINIDCKVLKNNFYSVCYSSELKNPIYGVSVLFGDLVDKNNIEKRYEF

SEQ ID NO: 47 - DNA, 755 bp, Helicobacter pylori
  CDS (223)...(672)
  sig_peptide (223)...(285)
  mat_peptide (286)...(672)
gattttaagcgcttgaaatagcccattttaatcaacaattaagcgactaaccattaaacttaagcgataaataagataaaatttaggatagctcaaatctttataaaaagaaaaggataaccccttacaaactttatttttaataaaaaatggcttatctcttctagcctactccccttattttttcttaaccctttagcggcagaagatgatggattttttatgggggtgagttatcaaacttctctagccgttcaaagggtggataactcagggcttaacgccagtcaagacgcatccacttacatccgccaaaacgctatcgctctagaatctgcggcagtgcctttagcctattatttagaagcgatgggccaacaaaccagagttttaatgcaaatgctctgccctgatccgtctaaaagatgtttgctctatgcggggggttataaaaacggatcaagtaatactaacggcgatacaggcaacaaccccccaagaggcaatgtcaatgccacctttgatatgcaatctttagtcaataatctaaacaaactcacccaactcatcggcgagactttaatccgtaaccctgaaaatctttctaacgccaaagtctttaacgtcaaatttggaaatcaaagcactgttattgcttttaccagagggtcttagacaaataccatgggacgcttttacaaatgacaatcaaccaacgcttttaaccacgctctgggtatttaccaaaaccctaa

SEQ ID NO: 48 - protein, 150 aa, Helicobacter pylori
MGVSYQTSLAVQRVDNSGLNASQDASTYIRQNAIALESAAVPLAYYLEAMGQQTRVLMQMLCPDPSKRCLLYAGGYKNGSSNTNGDTGNNPPRGNVNATFDMQSLVNNLNKLTQLIGETLIRNPENLSNAKVFNVKFGNQSTVIAFTRGS

SEQ ID NO: 49 - DNA, 29 bp, Helicobacter pylori
  misc_feature 4: n = A,T,C or G
gccngaaggatttattatgattaaaagaa

SEQ ID NO: 50 - DNA, 29 bp, Helicobacter pylori
  misc_feature 4: n = A,T,C or G
gccnaaggaataaattagaaagtgaagaa

SEQ ID NO: 51 - DNA, 25 bp, Helicobacter pylori
  misc_feature 4: n = A,T,C or G
gccnaaagggcgaaaatgagcaaga

SEQ ID NO: 52 - DNA, 28 bp, Helicobacter pylori
  misc_feature 4: n = A,T,C or G
gccnttttttaagaatcactttcttcgg

SEQ ID NO: 53 - DNA, 27 bp, Helicobacter pylori
  misc_feature 4: n = A,T,C or G
gccncgcattgatttgatgaataaacc

SEQ ID NO: 54 - DNA, 27 bp, Helicobacter pylori
  misc_feature 4: n = A,T,C or G
gccnctcactaaaaagcaatttttgag

SEQ ID NO: 55 - DNA, 28 bp, Helicobacter pylori
  misc_feature 4: n = A,T,C or G
gccntcacaatggataaaaacaacaaca

SEQ ID NO: 56 - DNA, 23 bp, Helicobacter pylori
  misc_feature 4: n = A,T,C or G
gccnctgtccaaatcagccaccc

SEQ ID NO: 57 - DNA, 25 bp, Helicobacter pylori
  misc_feature 4: n = A,T,C or G
gccnggaagaataatgctcgcttcc

SEQ ID NO: 58 - DNA, 25 bp, Helicobacter pylori
  misc_feature 4: n = A,T,C or G
gccnctattctccagggatatggcc

SEQ ID NO: 59 - DNA, 28 bp, Helicobacter pylori
  misc_feature 4: n = A,T,C or G
gccngaagggtgtatggtattaggaagc

SEQ ID NO: 60 - DNA, 29 bp, Helicobacter pylori
  misc_feature 4: n = A,T,C or G
gccncgttaaaactaaagttctattttta

SEQ ID NO: 61 - DNA, 28 bp, Helicobacter pylori
  misc_feature 4: n = A,T,C or G
gccnaatatatgggaacttaatgagaat

SEQ ID NO: 62 - DNA, 26 bp, Helicobacter pylori
  misc_feature 4: n = A,T,C or G
gccnatgtcatgtcaaactatgaagc

SEQ ID NO: 63 - DNA, 27 bp, Helicobacter pylori
  misc_feature 4: n = A,T,C or G
gccnaaaagggttttaaataatggctg

SEQ ID NO: 64 - DNA, 27 bp, Helicobacter pylori
  misc_feature 4: n = A,T,C or G
gccnaagattctaaaagggcttcaaat

SEQ ID NO: 65 - DNA, 38 bp, Helicobacter pylori
  misc_feature 4: n = A,T,C or G
gccngagagtagtggcagagtttatgctgattccgtta

SEQ ID NO: 66 - DNA, 26 bp, Helicobacter pylori
  misc_feature 4: n = A,T,C or G
gccnggcttaaactggaacggatttc

SEQ ID NO: 67 - DNA, 30 bp, Helicobacter pylori
  misc_feature 4: n = A,T,C or G
gccnatgaaaagatttgatttgtttttatc

SEQ ID NO: 68 - DNA, 26 bp, Helicobacter pylori
  misc_feature 4: n = A,T,C or G
gccnttaaatatcccaatcctgccac

SEQ ID NO: 69 - DNA, 35 bp, Helicobacter pylori
  misc_feature 4: n = A,T,C or G
gccntaaagtttgctaaaaagatggttttaatttc

SEQ ID NO: 70 - DNA, 28 bp, Helicobacter pylori
  misc_feature 4: n = A,T,C or G
gccncccatctttagaaatcaaccccca

SEQ ID NO: 71 - DNA, 32 bp, Helicobacter pylori
  misc_feature 4: n = A,T,C or G
gccncaataaaacaccaaaatgaatgagttac

SEQ ID NO: 72 - DNA, 30 bp, Helicobacter pylori
  misc_feature 4: n = A,T,C or G
gccngcatttaccccctaaaaactataaac

SEQ ID NO: 73 - DNA, 31 bp, Helicobacter pylori
  misc_feature 4: n = A,T,C or G
gccngtaaggaatgagatgataaagagttgg

SEQ ID NO: 74 - DNA, 28 bp, Helicobacter pylori
  misc_feature 4: n = A,T,C or G
gccnctaaactctggcttattgcgtatc

SEQ ID NO: 75 - DNA, 30 bp, Helicobacter pylori
  misc_feature 4: n = A,T,C or G
gccnataggaacaagcatgttttttaaaac

SEQ ID NO: 76 - DNA, 24 bp, Helicobacter pylori
  misc_feature 4: n = A,T,C or G
gccnctggcttattgcgtatcatc

SEQ ID NO: 77 - DNA, 32 bp, Helicobacter pylori
  misc_feature 4: n = A,T,C or G
gccngaaatcaaggagtttgtatgcaacagcg

SEQ ID NO: 78 - DNA, 32 bp, Helicobacter pylori
  misc_feature 4: n = A,T,C or G
gccncccaatacttttattgattcaccatttc

SEQ ID NO: 79 - DNA, 27 bp, Helicobacter pylori
  misc_feature 4: n = A,T,C or G
gccnatggtatttgacagaacaatcag

SEQ ID NO: 80 - DNA, 29 bp, Helicobacter pylori
  misc_feature 4: n = A,T,C or G
gccnttaggaatagcataacaaacaaacg

SEQ ID NO: 81 - DNA, 29 bp, Helicobacter pylori
  misc_feature 4: n = A,T,C or G
gccnatgttgaaatttaaatatggtttga

SEQ ID NO: 82 - DNA, 23 bp, Helicobacter pylori
  misc_feature 4: n = A,T,C or G
gccngagcctacaggttgcttgc

SEQ ID NO: 83 - DNA, 31 bp, Helicobacter pylori
  misc_feature 4: n = A,T,C or G
gccncaagcaaaaaaatgttcaataaaaggg

SEQ ID NO: 84 - DNA, 28 bp, Helicobacter pylori
  misc_feature 4: n = A,T,C or G
gccngtctaaattagaataagtgttgtt

SEQ ID NO: 85 - DNA, 30 bp, Helicobacter pylori
  misc_feature 4: n = A,T,C or G
gccntaaggaatgaagttgataaaatttgt

SEQ ID NO: 86 - DNA, 26 bp, Helicobacter pylori
  misc_feature 4: n = A,T,C or G
gccnggatttattgagctttcccctt

SEQ ID NO: 87 - DNA, 29 bp, Helicobacter pylori
  misc_feature 4: n = A,T,C or G
gccnatgttagaaaaattgattgaaagag

SEQ ID NO: 88 - DNA, 25 bp, Helicobacter pylori
  misc_feature 4: n = A,T,C or G
gccnttatgcgatagggggcgtatc

SEQ ID NO: 89 - DNA, 26 bp, Helicobacter pylori
  misc_feature 4: n = A,T,C or G
gccnatgcgtttattattgtggtggg

SEQ ID NO: 90 - DNA, 26 bp, Helicobacter pylori
  misc_feature 4: n = A,T,C or G
gccnttaaatttttagggaaagggta

SEQ ID NO: 91 - DNA, 30 bp, Helicobacter pylori
  misc_feature 4: n = A,T,C or G
gccnatgaaaaaatttttttctcaatcttt

SEQ ID NO: 92 - DNA, 29 bp, Helicobacter pylori
  misc_feature 4: n = A,T,C or G
gccnttattgatgaacattaaccattaaa

SEQ ID NO: 93 - DNA, 26 bp, Helicobacter pylori
  misc_feature 4: n = A,T,C or G
gccnatgaaaacctttaaaaacctgc

SEQ ID NO: 94 - DNA, 28 bp, Helicobacter pylori
  misc_feature 4: n = A,T,C or G
gccnttaaaactcatagcgtttttcaat

SEQ ID NO: 95 - DNA, 27 bp, Helicobacter pylori
  misc_feature 4: n = A,T,C or G
gccngatggattttttatgggggtgag

SEQ ID NO: 96 - DNA, 26 bp, Helicobacter pylori
  misc_feature 4: n = A,T,C or G
gccnatggtatttgtctaagaccctc

SEQ ID NO: 97 - DNA, 24 bp, Helicobacter pylori
aacctaatttgaaattcaaaccat

SEQ ID NO: 98 - DNA, 32 bp, Helicobacter pylori
aaattaggttttgtaggctttgccaataaatg

SEQ ID NO: 99 - DNA, 23 bp, Helicobacter pylori
taaaataaccaacagagtgatca

SEQ ID NO: 100 - DNA, 33 bp, Helicobacter pylori
ggttattttagtggatatttgggtttatagcga

SEQ ID NO: 101 - DNA, 19 bp, Helicobacter pylori
cgcctataaccgctccatt

SEQ ID NO: 102 - DNA, 32 bp, Helicobacter pylori
gttataggcgataaaggtttaacgcagctaag

SEQ ID NO: 103 - DNA, 21 bp, Helicobacter pylori
gcccttttgtttaggggttag

SEQ ID NO: 104 - DNA, 32 bp, Helicobacter pylori
acaaaagggctttttagagcatgtgagccatc

SEQ ID NO: 105 - DNA, 22 bp, Helicobacter pylori
actggagtgtggataaaactat

SEQ ID NO: 106 - DNA, 31 bp, Helicobacter pylori
acactccagtagatgctttcccggatatttc

SEQ ID NO: 107 - DNA, 26 bp, Helicobacter pylori
caccatacatgtatcctgcattaatg

SEQ ID NO: 108 - DNA, 33 bp, Helicobacter pylori
catgtatggtgtagcaaagaattttaaggaggc

SEQ ID NO: 109 - DNA, 22 bp, Helicobacter pylori
tgcgagatttaacctgttttca

SEQ ID NO: 110 - DNA, 32 bp, Helicobacter pylori
aaatctcgcagaaatctttcacaagcgagcaa

SEQ ID NO: 111 - DNA, 22 bp, Helicobacter pylori
acaaggataaaaaacgcgctaa

SEQ ID NO: 112 - DNA, 33 bp, Helicobacter pylori
ttatccttgttgctggcttggttttttttaatt

SEQ ID NO: 113 - DNA, 30 bp, Helicobacter pylori
aacttttctctatcccaattcgttacgctc

SEQ ID NO: 114 - DNA, 31 bp, Helicobacter pylori
ggatagagaaaagtttggcgtcaaaagttgg

SEQ ID NO: 115 - DNA, 22 bp, Helicobacter pylori
aagccgtattgtttgttttggc

SEQ ID NO: 116 - DNA, 34 bp, Helicobacter pylori
aatacggctttaaagctatagaaaatttaaacgc

SEQ ID NO: 117 - DNA, 28 bp, Helicobacter pylori
gacttctaaagcgtcctttttttcttta

SEQ ID NO: 118 - DNA, 28 bp, Helicobacter pylori
ctttagaagtcattaaacaaagaggggt

SEQ ID NO: 119 - DNA, 26 bp, Helicobacter pylori
agattttgttttgagcgttagaaatg

SEQ ID NO: 120 - DNA, 32 bp, Helicobacter pylori
caaaatctataaactcaatcaagtcaaaaatg

SEQ ID NO: 121 - DNA, 24 bp, Helicobacter pylori
tggaatattgtgatccacgccatc

SEQ ID NO: 122 - DNA, 37 bp, Helicobacter pylori
gaatattccaaaagccgttttttattacagaagaggg

SEQ ID NO: 123 - DNA, 22 bp, Helicobacter pylori
tgaagtcttgcgatttttgctt

SEQ ID NO: 124 - DNA, 30 bp, Helicobacter pylori
caagacttcaaaaaagaaggagcggttgcc

SEQ ID NO: 125 - DNA, 27 bp, Helicobacter pylori
aagcttttcattatcttccccataagc

SEQ ID NO: 126 - DNA, 29 bp, Helicobacter pylori
tgaaaagcttttagcgaagcgatcaagcc

SEQ ID NO: 127 - DNA, 20 bp, Helicobacter pylori
gaaaagccaccccgcttatt

SEQ ID NO: 128 - DNA, 32 bp, Helicobacter pylori
gtggcttttcaaaaagagtgggtgcaacaatt

SEQ ID NO: 129 - DNA, 21 bp, Helicobacter pylori
aaaccccactcttatcatcgg

SEQ ID NO: 130 - DNA, 30 bp, Helicobacter pylori
agtggggtttttttagggggtgggtatgct

SEQ ID NO: 131 - DNA, 22 bp, Helicobacter pylori
taagtccatacgatagcctatg

SEQ ID NO: 132 - DNA, 34 bp, Helicobacter pylori
tatggaacttagaacattttaacacgctctatta

SEQ ID NO: 133 - DNA, 23 bp, Helicobacter pylori
gcattttcattcattctttggac

SEQ ID NO: 134 - DNA, 32 bp, Helicobacter pylori
atgaaaatgcacgcccaaataataaggaagta

SEQ ID NO: 135 - DNA, 22 bp, Helicobacter pylori
tgaacacatagcctaaaaccac

SEQ ID NO: 136 - DNA, 31 bp, Helicobacter pylori
tatgtgttcatgaaagagttgtggcacatgc

SEQ ID NO: 137 - DNA, 27 bp, Helicobacter pylori
caatacccaccacaataataaacgcat

SEQ ID NO: 138 - DNA, 33 bp, Helicobacter pylori
gtgggtattggtattatcgctctttttaaatcc

SEQ ID NO: 139 - DNA, 19 bp, Helicobacter pylori
tggccagtagcgcgttcat

SEQ ID NO: 140 - DNA, 34 bp, Helicobacter pylori
ctactggccatggatggcaatggcgtttttttag

SEQ ID NO: 141 - DNA, 21 bp, Helicobacter pylori
tagcgatcaggctaaaacaga

SEQ ID NO: 142 - DNA, 29 bp, Helicobacter pylori
ctgatcgctatgagttggctccaagcgga

SEQ ID NO: 143 - DNA, 19 bp, Helicobacter pylori
ggcactgccgcagattcta

SEQ ID NO: 144 - DNA, 33 bp, Helicobacter pylori
cggcagtgcctttagcctattatttagaagcga

SEQ ID NO: 145 - DNA, 30 bp, Helicobacter pylori
gccgagctctatcgtatggacttagaacat

SEQ ID NO: 146 - DNA, 34 bp, Helicobacter pylori
gccctcgagattagaataagtgttgtttaaaatc